(19) United States
(12) Patent Application Publication
(10) Pub. No.: US 2014/0206574 A1
(43) Pub. Date: Jul. 24, 2014

(54) Methods and Compositons for the Treatment and Diagnosis of Cancer

(71) Applicants: Karen Chapman, Mill Valley, CA (US); Joseph Wagner, San Ramon, CA (US); Michael West, Mill Valley, CA (US); Markus Daniel Lacher, Lafayette, CA (US); Jennifer Lorie Kidd, Alameda, CA (US); Maria J. Prendes, Santa Cruz, CA (US)

(72) Inventors: Karen Chapman, Mill Valley, CA (US); Joseph Wagner, San Ramon, CA (US); Michael West, Mill Valley, CA (US); Markus Daniel Lacher, Lafayette, CA (US); Jennifer Lorie Kidd, Alameda, CA (US); Maria J. Prendes, Santa Cruz, CA (US)

(21) Appl. No.: 14/240,698

(22) PCT Filed: Aug. 31, 2012
(86) PCT No.: PCT/US12/53472

§ 371 Date: Feb. 25, 2014

Related U.S. Application Data
(60) Provisional application No. 61/529,500, filed on Aug. 31, 2011.
(60) Provisional application No. 61/542,403, filed on Oct. 3, 2011.

(51) Int. Cl. G01N 33/574; C12Q 1/68

(52) U.S. Cl. CPC G01N 33/57488; C12Q 1/6886
USPC 506/9; 435/6.11; 436/501; 435/7.4; 435/7.23; 435/6.12; 435/7.92; 506/18; 506/16; 435/6.14

(57) ABSTRACT

The invention relates to methods of detecting cancer in a sample obtained from a subject. The invention also provides kits and reagents for detecting cancer as well as therapeutics and methods of treating cancer.

This application claims priority U.S. Provisional Application No. 61/529,500, filed Aug. 31, 2011, the entire contents of which are hereby incorporated by reference.

### FIELD OF THE INVENTION

The field of the invention relates to cancer and the diagnosis and treatment of cancer.

### BACKGROUND

Early detection of cancer can impact treatment outcomes and disease progression. Typically, cancer detection relies on diagnostic information obtained from biopsy, x-rays, CAT scans, NMR and the like. These procedures may be invasive, time consuming and expensive. Moreover, they have limitations with regard to sensitivity and specificity. There is a need in the field of cancer diagnostics for a highly specific, highly sensitive, rapid, inexpensive, and relatively non-invasive method of diagnosing cancer. Various embodiments of the invention described below meet this need as well as other needs existing in the field of diagnosing and treating cancer.

### SUMMARY OF THE INVENTION

Embodiments of the disclosure provide methods of diagnosis, prognosis and treatment of cancer. Other embodiments provide compositions relating to the diagnosis, prognosis and treatment of cancer.

In certain embodiments the invention provides a method of detecting cancer in a subject comprising a) obtaining a sample from a subject; b) contacting the sample obtained from the subject with one or more agents that detect one or more markers expressed by a cancer cell c) contacting a non-cancerous cell with the one or more agents from b); and d) comparing the expression level of the marker in the sample obtained from the subject with the expression level in the non-cancerous cell, wherein a higher level of expression of the marker in the sample compared to the non-cancerous cell indicates that the subject has cancer.

In certain embodiments the invention provides a method of detecting cancer in a subject comprising a) obtaining a sample from a subject; b) contacting the sample obtained from the subject with one or more agents that detect expression of at least one of the markers listed in Table 1; c) contacting a non-cancerous cell, with the one or more agents from b); and d) comparing the expression level of one or more of the markers listed in Table 1 in the sample obtained from the subject with the expression level of one or more of the markers listed in Table 1 in the non-cancerous cell, wherein a higher level of expression of one or more of the markers listed in Table 1 in the sample obtained from the subject compared to the non-cancerous cell indicates that the subject has cancer.

In some embodiments the invention provides a method of detecting cancer in a subject comprising a) obtaining a sample from a subject b) contacting the sample obtained from the subject with one or more agents that detect expression of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (NEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21 orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1 orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof; c) contacting a non-cancerous cell with the one or more agents from b); and d) comparing the expression level of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof in the non-cancerous cell, wherein a higher level of expression of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof in the sample obtained from the subject compared to the non-cancerous cell indicates that the subject has cancer.

In other embodiments the invention provides a method of detecting cancer in a subject comprising a) obtaining a sample from a subject b) contacting the sample obtained from the subject with one or more agents that detect expression of a panel of markers encoded by the genes GNGT1, C12orf56, COL10A1, SLC35D3, snaR-A, SBK1, DSCR8, CELSR3 or a complement thereof; c) contacting a non-cancerous cell, with the one or more agents from b); and d) comparing the expression level of the panel of markers encoded for by the genes GNGT1, C12orf56, COL10A1, SLC35D3, snaR-A, SBK1, DSCR8, CELSR3 or a complement thereof in the sample obtained from the subject with the expression level of the panel of markers encoded for by the genes GNGT1, C12orf56, COL10A1, SLC35D3, snaR-A, SBK1, DSCR8, CELSR3 or a complement thereof in the non-cancerous cell, wherein a higher level of expression of the panel of markers encoded for by genes GNGT1, C12orf56, COL10A1, SLC35D3, snaR-A, SBK1, DSCR8, CELSR3 or a complement thereof in the sample compared to the non-cancerous cell in wherein a higher level of expression of the panel of markers encoded by genes GNGT1, C12orf56, COL10A1, SLC35D3, snaR-A, SBK1, DSCR8, CELSR3 or a complement thereof in the sample compared to the non-cancerous cell indicates that the subject has cancer that the subject has cancer.

In some embodiments the invention provides a method of detecting cancer in a subject comprising a) obtaining a sample from a subject b) contacting the sample obtained from the subject with one or more agents that detect expression of one or more of the markers encoded by genes chosen from GNGT1, C12orf56, COL10A1, SLC35D3, snaR-A, SBK1, DSCR8, CELSR3 or a complement thereof; c) contacting a non-cancerous cell with the one or more agents from b); and d) comparing the expression level of one or more of the markers encoded by genes chosen from GNGT1, C12orf56, COL10A1, SLC35D3, snaR-A, SBK1, DSCR8, CELSR3 or a complement thereof in the sample obtained from the subject with the expression level of one or more of the markers encoded by genes chosen from GNGT1, C12orf56, COL10A1, SLC35D3, snaR-A, SBK1, DSCR8, CELSR3 or a complement thereof in the non-cancerous cell, wherein a higher level of expression of one or more of the markers encoded by genes chosen from GNGT1, C12orf56, COL10A1, SLC35D3, snaR-A, SBK1, DSCR8, CELSR3 or a complement thereof in the sample obtained from the subject compared to the non-cancerous cell indicates that the subject has cancer.

In further embodiments the invention provides a method of detecting cancer cells in a sample comprising a) obtaining a sample b) contacting the sample obtained in a) with one or more agents that detect expression of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof; c) contacting a non-cancerous cell with the one or more agents from b); and d) comparing the expression level of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof in the sample obtained in a) with the expression level of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A 13 (ANXA13), Homo sapiens apolipoprotein B mRNA editing enzyme, catalytic polypeptide 1 (APOBEC1), Homo sapiens chromosome 1 open reading frame 110 (C1 orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof in the non-cancerous cell, wherein a higher level of expression of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (L1N28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof in the sample compared to the non-cancerous cell indicates that the sample contains cancer cells. The sample may be an in vitro sample or an in vivo sample, or derived from an in vivo sample.

In certain embodiments the invention provides a method of detecting cancer in a sample comprising a) contacting the sample with one or more agents that detect expression of at least one of the markers chosen from SLC35D, NMU, MMP12, MMP11, MMP7, DSCR8, COL10A, C2orf70, C12orf56, ASCL1, WNT10A, OLFM4, PI3, IL8, EPYC, and CXCL10; c) contacting a non-cancerous cell, with the one or more agents from b); and d) comparing the expression level of one or more of the markers chosen from GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the sample with the expression level of one or more of the markers chosen from GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the non-cancerous cell, wherein a higher level of expression of one or more of the markers in the sample chosen from GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the sample compared to the non-cancerous cell indicates that the sample has cancer cells. The cancer cells may be from lung, bladder, breast, kidney, and/or pancreatic cancer for example.

With regard to the embodiments described in the preceding paragraphs, the sample may be any sample as described infra, for example, a bodily fluid, such as blood, serum or urine. The sample may be a cellular sample or the extract of a cellular sample. The sample may be a tissue sample. Nucleic acids and/or proteins may be isolated from the sample. Nucleic acids such as RNA may be transcribed into cDNA. The agent may be one or more molecules that bind specifically to one or more proteins expressed by the cancer cell or one or more nucleic acids expressed by the cell. For example, the agent may be a protein such as an antibody that binds specifically to the protein expressed by one of the marker genes identified infra. The agent may be one or more nucleic acids that hybridize to a nucleic acid expressed by the cancer cell. The nucleic acid expressed by the cancer cell may be an RNA molecule, e.g. an mRNA molecule. The nucleic acid molecule that hybridizes to the nucleic acid expressed by the cancer cell may be a DNA molecule, such as a DNA probe.

In still other embodiments the invention provides a composition of matter useful in distinguishing a cancer cell from a non-cancerous cell comprising one or more molecules that specifically bind to a molecule expressed at higher levels on a cancer cell compared to a non-cancer cell. As an example, the composition may comprise a protein, that binds to one or more molecules expressed by the cancer cell at higher levels compared to the non-cancer cell. As another example, the composition may comprise a nucleic acid that binds to one or more molecules expressed by the cancer cell at higher levels compared to the non-cancer cell.

In some embodiments the invention provides a composition of matter comprising a protein, such as an antibody, that specifically binds to a molecule expressed by a cancer cell chosen from the markers encoded by the sequences listed in Table 1. The molecule expressed by the cancer cell may be expressed by the cancer cell at a level that is higher than the level expressed by a non-cancerous cell.

In further embodiments the invention provides a composition of matter comprising a plurality of proteins, such as a plurality antibodies, that specifically binds to a panel of molecules expressed by a cancer cell wherein the panel of markers comprises molecule encoded by the genes GNGT1, C12orf56, COL10A1, SLC35D3, snaR-A, SBK1, DSCR8, CELSR3 or a complement thereof. The panel of markers may be expressed at a level that is higher than the level of the panel of markers in a non-cancerous cell.

In certain embodiments the invention provides a composition of matter comprising a protein, such as an antibody, that specifically binds to a molecule expressed by a cancer cell chosen from a molecule encoded by one or more of the genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (L1N28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1 orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof or a complement thereof. The molecule expressed by the cancer cell may be expressed by the cancer cell at level that is higher than the level expressed by a non-cancerous cell.

In other embodiments the invention provides a composition of matter comprising a nucleic acid that specifically binds to a molecule, such as an mRNA molecule, expressed by a cancer cell wherein the molecule is chosen from a marker encoded for by the genes listed in Table 1. The molecule expressed by the cancer cell may be expressed by the cancer cell at level that is higher than the level expressed by a non-cancerous cell.

In other embodiments the invention provides a composition of matter comprising a nucleic acid that specifically binds to a molecule, such as an mRNA molecule, expressed by a cancer cell wherein the molecule is encoded for by a gene disclosed infra, e.g. a gene disclosed under the heading Cancer Associated Sequences, or a complement thereof. The molecule expressed by the cancer cell may be expressed by the cancer cell at level that is higher than the level expressed by a non-cancerous cell.

In still further embodiments the invention provides a method of determining if a cancer in a subject is advancing comprising a) measuring the expression level of one or more markers associated with cancer at a first time point; b) measuring the expression level of the one or more markers measured in a) at a second time point, wherein the second time point is subsequent to the first time point; and c) comparing the expression level measured in a) and b), wherein an increase in the expression level of the one or more markers in b) compared to a) indicates that the subject's cancer is advancing.

In some embodiments the invention provides a method of determining if a cancer in a subject is advancing comprising a) measuring the expression level of one or more markers listed in Table 1 at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's cancer is advancing.

In other embodiments the invention provides a method of determining if a cancer in a subject is advancing comprising a) measuring the expression level of one or more markers encoded by genes chosen from a gene disclosed infra, e.g., a gene disclosed infra under the heading Cancer Associated Sequences, or a complement thereof at a first time point; b) measuring the expression level of the one or more markers measured in a) at a second time point, wherein the second time point is subsequent to the first time point; and c) comparing the expression level measured in a) and b), wherein an increase in the expression level of the one or more markers at the second time point compared to the first time point indicates that the subject's cancer is advancing.

In some embodiments the invention provides antigens (i.e. cancer-associated polypeptides) associated with cancer as targets for diagnostic and/or therapeutic antibodies. In some embodiments, the antigen may be chosen from a protein encoded by, a gene listed in Table 1, a fragment thereof, or a combination of proteins encoded by a gene listed in Table 1.

In some embodiments the invention provides antigens (i.e. cancer-associated polypeptides) associated with cancer as targets for diagnostic and/or therapeutic antibodies. In some embodiments, the antigen may be chosen from a protein encoded by, a gene chosen from a gene disclosed infra, e.g. under the heading Cancer Associated Genes, a fragment thereof, or a combination of proteins encoded by a gene (or fragments thereof) chosen from a gene disclosed infra, e.g. a gene disclosed under the heading Cancer Associated Sequences.

In yet other embodiments the invention provides a method of eliciting an immune response to a cancer cell comprising contacting a subject with a protein or protein fragment that is expressed by a cancer cell thereby eliciting an immune response to the cancer cell. As an example the subject may be contacted intravenously or intramuscularly with protein or protein fragment.

In further embodiments the invention provides a method of eliciting an immune response to a cancer cell comprising contacting a subject with one or more proteins or protein fragments that is encoded by a gene chosen from the genes listed in Table 1, thereby eliciting an immune response to a cancer cell. As an example the subject may be contacted with the protein or the protein fragment intravenously or intramuscularly.

In still other embodiments the invention provides a method of eliciting an immune response to a cancer cell comprising contacting a subject with one or more proteins or protein fragments that is encoded by a gene chosen from a gene disclosed infra, e.g., a gene disclosed under the heading Cancer Associated Sequences, thereby eliciting an immune response to a cancer cell. As an example the subject may be contacted with the protein or protein fragment intravenously or intramuscularly.

In yet other embodiments the invention provides a kit for detecting cancer cells in a sample. The kit may comprise one or more agents that detect expression of any the cancer associated sequences disclosed infra. The kit may include agents that are proteins and/or nucleic acids for example. In one embodiment the kit provides a plurality of agents. The agents may be able to detect the panel of markers encoded by the genes comprising GNGT1, C12orf56, COL10A1, SLC35D3, snaR-A, SBK1, DSCR8, CELSR3 or a complement thereof.

In still other embodiments the invention provides a kit for detecting cancer in a sample comprising a plurality of agents that specifically bind to a molecule encoded for by the genes SLC35D, NMU, MMP12, MMP11, MMP7, DSCR8, COL10A, C2orf70, C12orf56, ASCL1, WNT10A, OLFM4, PI3, IL8, EPYC, and CXCL10.

In other embodiments the invention provides a kit for detection of cancer in a sample obtained from a subject. The kit may comprise one or more agents that bind specifically to a molecule expressed specifically by a cancer cell. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The kit may further contain a positive control (e.g. one or more cancerous cells; or specific known quantities of the molecule expressed by the cancer cell) and a negative control (e.g. a tissue or cell sample that is non-cancerous).

In some embodiments the invention provides a kit for the detection of cancer comprising one or more agents that specifically bind one or more markers encoded by genes chosen from a gene disclosed infra., e.g., a gene disclosed under the heading Cancer Associated Sequences. The agent may be a protein, such as an antibody. Alternatively, the agent may be a nucleic such as a DNA molecule or an RNA molecule. The kit may comprise one or more containers and instructions for determining if the sample is positive for cancer. The kit may optionally contain one or more multiwell plates, a detectable substance such as a dye, a radioactively labeled molecule, a chemiluminescently labeled molecule and the like. The kit may further contain a positive control (e.g. one or more cancerous cells; or specific known quantities of the molecule expressed by the cancer cell) and a negative control (e.g. a tissue or cell sample that is non-cancerous). As an example the kit may take the form of an ELISA or a DNA microarray.

Some embodiments are directed to a method of treating cancer in a subject, the method comprising administering to a subject in need thereof a therapeutic agent modulating the activity of a cancer associated protein, wherein the cancer associated protein is encoded by gene listed in Table 1, homologs thereof, combinations thereof, or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody.

Some embodiments herein are directed to a method of treating cancer in a subject, the method comprising administering to a subject in need thereof a therapeutic agent modulating the activity of a cancer associated protein, wherein the cancer associated protein is encoded by gene chosen from a gene disclosed infra, e.g. a gene disclosed under the heading Cancer Associated Sequences, and/or homologs thereof, and/or combinations thereof, and/or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody.

In some embodiments, a method of treating cancer in a subject may comprise administering to a subject in need thereof a therapeutic agent that modulates the expression of one or more genes chosen from those listed in Table 1, fragments thereof, homologs thereof, and/or complements thereof.

In some embodiments, a method of treating cancer in a subject may comprise administering to a subject in need thereof a therapeutic agent that modulates the expression of one or more genes chosen from a gene disclosed infra, e.g. a gene disclosed under the heading Cancer Associated Sequences, fragments thereof, homologs thereof, and or compliments thereof.

In further embodiments, the invention provides a method of treating cancer may comprising a gene knockdown of one or more genes listed in Table 1 fragments thereof, homologs thereof, and or compliments thereof. In some embodiments, a method of treating cancer may comprise treating cells to knockdown or inhibit expression of a gene encoding an mRNA of one or more genes chosen from those listed n Table 1, fragments thereof, homologs thereof, and or compliments thereof.

In other embodiments, a method of treating cancer may comprise gene knockdown of one or more genes selected from a gene disclosed infra, e.g., a gene disclosed under the heading Cancer Associated Sequences. In some embodiments, a method of treating cancer may comprise treating cells to knockdown or inhibit expression of a gene encoding an mRNA of one or more genes chosen from a gene disclosed infra, e.g. a gene disclosed under the heading Cancer Associated sequences.

In still other embodiments, the present invention provides methods of screening a drug candidate for activity against cancer, the method comprising: (a) contacting a cell that expresses one or more cancer associated genes chosen from those listed in Table 1 with a drug candidate; (b) detecting an effect of the drug candidate on expression of the one or more cancer associated genes in the cell from a); and (c) comparing the level of expression of one or more of the genes recited in a) in the absence of the drug candidate to the level of expression of the one or more genes in the presence of the drug candidate; wherein a decrease in the expression of the cancer associated gene in the presence of the drug candidate indicates that the candidate has activity against cancer.

In further embodiments, the present invention provides methods of screening a drug candidate for activity against cancer, the method comprising: (a) contacting a cell that expresses one or more cancer associated genes chosen from a gene disclosed infra., e.g., a gene disclosed under the heading Cancer Associated Sequences, with a drug candidate; (b) detecting an effect of the drug candidate on an expression of the one or more cancer associated genes in the cell from a); and (c) comparing the level of expression of one or more of the genes recited in a) in the absence of the drug candidate to the level of expression in the presence of the drug candidate; wherein a decrease in the expression of the cancer associated gene in the presence of the drug candidate indicates that the candidate has activity against cancer.

In some embodiments, the present invention provides methods of visualizing a cancer tumor in a subject comprising a) targeting one or more cancer associated proteins with a labeled molecule that binds specifically to the cancer tumor, wherein the cancer associated protein is selected from a protein encoded for by one or more genes chosen from those listed in Table 1; and b) detecting the labeled molecule, wherein the labeled molecule visualizes the tumor in the subject. Visualization may be done in vivo, or in vitro.

In still other embodiments, the present invention provides methods of visualizing a cancer tumor in a subject comprising a) targeting one or more cancer associated proteins with a labeled molecule that binds specifically to the cancer tumor, wherein the cancer associated protein is selected from a protein encoded for by one or more genes chosen from a gene disclosed infra, e.g., a gene disclosed under the heading Cancer Associated Sequences; and b) detecting the labeled molecule, wherein the labeled molecule visualizes the tumor in the subject. Visualization may be done in vivo or in vitro.

### DESCRIPTION OF DRAWINGS

For a fuller understanding of the nature and advantages of the present invention, reference should be had to the following detailed description taken in connection with the accompanying drawings, in which:

FIG. 1 shows the expression of GNGT1 in normal cells and tissues versus tumors.

FIG. 2 shows the expression of C12orf56 in normal cells and tissues versus tumors.

FIG. 3 shows the expression of COL10A1 in normal cells and tissues versus tumors.

FIG. 4 shows the expression of SLC35D3 in normal cells and tissues versus tumors.

FIG. 5 shows the expression of snaR-A in normal cells and tissues versus tumors.

FIG. 6 shows the expression of SBK1 in normal cells and tissues versus tumors.

FIG. 7 shows the expression of DSCR8 in normal cells and tissues versus tumors.

FIG. 8 shows the expression of CELSR3 in normal cells and tissues versus tumors.

FIG. 9 shows the expression of PPEF1 in normal cells and tissues versus tumors.

FIG. 10 shows serum expression levels of COL10A1 in serum from breast cancer subjects compared to normal donor serum.

FIG. 11 shows serum expression levels of COL10A1 in serum from colon cancer subjects compared to normal donor serum.

FIG. 12 shows serum expression levels of COL10A1 in serum from kidney cancer subjects compared to normal donor serum.

FIG. 13 shows serum expression levels of COL10A1 in serum from lung cancer subjects compared to normal donor serum.

FIG. 14 shows serum expression levels of COL10A1 in serum from bladder cancer subjects compared to normal donor serum.

FIG. 15 shows serum expression levels of CXCL10 in serum from breast cancer subjects compared to normal donor serum.

FIG. 16 shows serum expression levels of EPYC in serum from breast cancer subjects compared to normal donor serum.

FIG. 17 shows serum expression levels of IL8 in serum from breast cancer subjects compared to normal donor serum.

FIG. 18 shows serum expression levels of LAMC2 in serum from pancreatic cancer subjects compared to normal donor serum.

FIG. 19 shows serum expression levels of PI3 in serum from colon cancer subjects compared to normal donor serum.

FIG. 20 shows serum expression levels of MMP7 in serum from breast cancer subjects compared to normal donor serum.

FIG. 21 shows serum expression levels of MMP7 in serum from colon cancer subjects compared to normal donor serum.

FIG. 22 shows serum expression levels of MMP7 in serum from pancreatic cancer subjects compared to normal donor serum.

FIG. 23 shows serum expression levels of MMP11 in serum from colon cancer subjects compared to normal donor serum and subjects with benign tumors.

FIG. 24 shows serum expression levels of MMP11 in serum from pancreatic cancer subjects compared to normal donor serum.

FIG. 25 shows serum expression levels of MMP11 in serum from breast cancer subjects compared to normal donor serum.

FIG. 26 shows serum expression levels of MMP11 in serum from bladder cancer subjects compared to normal donor serum.

FIG. 27 shows serum expression levels of MMP12 in serum from breast cancer subjects compared to normal donor serum and subjects with benign breast tumors.

FIG. 28 shows serum expression levels of MMP12 in serum from colon cancer subjects compared to normal donor serum.

FIG. 29 shows serum expression levels of MMP12 in serum from pancreatic cancer subjects compared to normal donor serum.

FIG. 30 shows serum expression levels of NMU in serum from breast cancer subjects compared to normal donor serum.

FIG. 31 shows serum expression levels of NMU in serum from colon cancer subjects compared to normal donor serum.

FIG. 32 shows serum expression levels of OLFM4 in serum from colon cancer subjects compared to normal donor serum.

FIG. 33 shows serum expression levels of WNT10A in serum from breast cancer subjects compared to normal donor serum.

FIG. 34 shows serum expression levels of WNT10A in serum from colon cancer subjects compared to normal donor serum.

FIG. 35 shows expression levels of AMH_{—}1038 in various normal tissue and cancer tissue.

FIG. 36 shows expression levels of ASCL1_{—}1095 in breast tumors, tissue adjacent to breast tumors, and normal breast tissue.

FIG. 37 shows expression levels of C12orf56 in various normal tissue and cancer tissue.

FIG. 38 shows expression levels of C2orf70_{—}1010 in various normal tissue and cancer tissue.

FIG. 39 shows expression levels of COL10A in various normal tissue and cancer tissue.

FIG. 40 shows expression levels of COL10A in various normal tissue and cancer tissue.

FIG. 41 shows expression levels of COL10A in various normal tissue and cancer tissue.

FIG. 42 shows expression levels of COL10A in various normal tissue and cancer tissue.

FIG. 43 shows expression levels of COL10A in breast tumors, tissue adjacent to breast tumors, and normal breast tissue.

FIG. 44 shows expression levels of DSCR_{—}1066 in breast tumors, tissue adjacent to breast tumors, and normal breast tissue.

FIG. 45 shows expression levels of DSCR8 in various normal tissue and cancer tissue.

FIG. 46 shows expression levels of MMP11 in breast tumors, tissue adjacent to breast tumors, and normal breast tissue.

FIG. 47 shows expression levels of MMP12 in bladder tumors, tissue adjacent to breast tumors, and normal bladder tissue.

FIG. 48 shows expression levels of NMU in thyroid tumors, tissue adjacent to breast tumors, and normal thyroid tissue.

FIG. 49 shows expression levels of SLC35D in colon tumors and normal colon tissue.

FIG. 50 shows expression of POTE in breast tumor and normal breast tissue as measured by immunocytochemistry.

FIG. 51 shows expression of MMP11 in breast tumor and normal breast tissue as measured by immunocytochemistry.

FIG. 52 shows expression levels of L1TD1 in colon tumors and normal colon tissue.

FIG. 53 shows expression levels of APOBEC1 in colon tumors and normal colon tissue.

### I. DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference in their entirety. Nothing herein is to be construed as an admission that the invention is not entitled to antedate such disclosure by virtue of prior invention.

As used herein, the singular forms “a,” “an,” and “the” include plural reference unless the context clearly dictates otherwise. Thus, for example, reference to a “therapeutic” is a reference to one or more therapeutics and equivalents thereof known to those skilled in the art, and so forth.

As used herein, the term “about” means plus or minus 10% of the numerical value of the number with which it is being used. Therefore, about 50% means in the range of 45% to 55%.

“Agent” as used herein refers to a molecule that specifically binds to a cancer associated sequence or a molecule encoded for by a cancer associated sequence. Examples of agents include nucleic acid molecules, such as DNA and proteins such as antibodies. The agent may be linked with a label or detectible substance as described infra.

“Administering,” when used in conjunction with a therapeutic, means to administer a therapeutic directly into or onto a target tissue or to administer a therapeutic to a patient whereby the therapeutic positively impacts the tissue to which it is targeted. Thus, as used herein, the term “administering,” when used in conjunction with a therapeutic, can include, but is not limited to, providing the therapeutic into or onto the target tissue; providing the therapeutic systemically to a patient by, e.g., intravenous injection whereby the therapeutic reaches the target tissue; providing the therapeutic in the form of the encoding sequence thereof to the target tissue (e.g., by so-called gene-therapy techniques). “Administering” a composition may be accomplished by oral administration, intravenous injection, intraperitoneal injection, intramuscular injection, subcutaneous injection, transdermal diffusion or electrophoresis, local injection, extended release delivery devices including locally implanted extended release devices such as bioerodible or reservoir-based implants, as protein therapeutics or as nucleic acid therapeutic via gene therapy vectors, topical administration, or by any of these methods in combination with other known techniques. Such combination techniques include, without limitation, heating, radiation and ultrasound.

The term “amplify” is used to mean creating an amplification product which may include, for example, additional target molecules, or target-like molecules or molecules complementary to the target molecule, which molecules are created by virtue of the presence of the target molecule in the sample. In the situation where the target is a nucleic acid, an amplification product can be made enzymatically with DNA or RNA polymerases or reverse transcriptases, or any combination thereof.

The term “animal,” “patient” or “subject” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” “patient” or “animal” refers to a male. In some embodiments, the term “subject,” “patient” or “animal” refers to a female.

The term “biological sources” as used herein refers to the sources from which the target polynucleotides and/or proteins or peptides may be derived. The source can be of any form of “sample” as described above, including but not limited to, cell, tissue or fluid. “Different biological sources” can refer to different cells/tissues/organs of the same individual, or cells/tissues/organs from different individuals of the same species, or cells/tissues/organs from different species.

The term “capture reagent” refers to a reagent, for example an antibody or antigen binding protein, capable of binding a target molecule or analyte to be detected in a sample.

The term “gene expression result” refers to a qualitative and/or quantitative result regarding the expression of a gene or gene product. The gene expression result can be an amount or copy number of the gene, the RNA encoded by the gene, the mRNA encoded by the gene, the protein product encoded by the gene, or any combination thereof. The gene expression result can also be normalized or compared to a standard. The gene expression result can be used, for example, to determine if a gene is expressed, overexpressed, or differentially expressed in two or more samples.

The term “homology,” as used herein, refers to a degree of complementarity. There may be partial homology or complete homology. The word “identity” may substitute for the word “homology.” A partially complementary nucleic acid sequence that at least partially inhibits an identical sequence from hybridizing to a target nucleic acid is referred to as “substantially homologous.” The inhibition of hybridization of the completely complementary nucleic acid sequence to the target sequence may be examined using a hybridization assay (Southern or northern blot, solution hybridization, and the like) under conditions of reduced stringency. A substantially homologous sequence or hybridization probe will compete for and inhibit the binding of a completely homologous sequence to the target sequence under conditions of reduced stringency. This is not to say that conditions of reduced stringency are such that non-specific binding is permitted, as reduced stringency conditions require that the binding of two sequences to one another be a specific (i.e., a selective) interaction. The absence of non-specific binding may be tested by the use of a second target sequence which lacks even a partial degree of complementarity (e.g., less than about 30% homology or identity). In the absence of non-specific binding, the substantially homologous sequence or probe will not hybridize to the second non-complementary target sequence.

As used herein, the term “hybridization” or “hybridizing” refers to hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. “Complementary,” as used herein in reference to nucleic acid molecules refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that a nucleic acid sequence need not be 100% complementary to that of its target nucleic acid to be specifically hybridizable. A nucleic acid compound is specifically hybridizable when there is binding of the molecule to the target, and there is a sufficient degree of complementarity to avoid non-specific binding of the molecule to non-target sequences under conditions in which specific binding is desired, i.e., under physiological conditions in the case of in vivo assays or therapeutic treatment, and in the case of in vitro assays, under conditions in which the assays are performed.

The term “inhibiting” includes the administration of a compound of the present disclosure to prevent the onset of the symptoms, alleviating the symptoms, or eliminating the disease, condition or disorder. The term “inhibiting” may also refer to lowering the expression level of gene, such as a gene encoding a cancer associated sequence. Expression level of RNA and/or protein may be lowered.

The term “label” and/or detectible substance refers to a composition capable of producing a detectable signal indicative of the presence of the target polynucleotide in an assay sample. Suitable labels include radioisotopes, nucleotide chromophores, enzymes, substrates, fluorescent molecules, chemiluminescent moieties, magnetic particles, bioluminescent moieties, and the like. As such, a label is any composition detectable by a device or method, such as, but not limited to, a spectroscopic, photochemical, biochemical, immunochemical, electrical, optical, chemical detection device or any other appropriate device. In some embodiments, the label may be detectable visually without the aid of a device. The term “label” is used to refer to any chemical group or moiety having a detectable physical property or any compound capable of causing a chemical group or moiety to exhibit a detectable physical property, such as an enzyme that catalyzes conversion of a substrate into a detectable product. The term “label” also encompasses compounds that inhibit the expression of a particular physical property. The label may also be a compound that is a member of a binding pair, the other member of which bears a detectable physical property.

As used herein, “microarray” refers to a linear or two-dimensional array of, for example, discrete regions, each having a defined area, formed on the surface of a solid support. The density of the discrete regions on a microarray is determined by the total numbers of target polynucleotides to be detected on the surface of a single solid phase support, preferably at least about 50/cm², more preferably at least about 100/cm², even more preferably at least about 500/cm², and still more preferably at least about 1,000/cm². As used herein, a DNA microarray is an array of oligonucleotide primers placed on a chip or other surfaces used to identify, amplify, detect, or clone target polynucleotides. Since the position of each particular group of primers in the array is known, the identities of the target polynucleotides can be determined based on their binding to a particular position in the microarray.

As used herein, the term “naturally occurring” refers to sequences or structures that may be in a form normally found in nature. “Naturally occurring” may include sequences in a form normally found in any animal.

As used herein, the use of “nucleic acid,” “polynucleotide” or “oligonucleotide” or equivalents herein means at least two nucleotides covalently linked together. In some embodiments, an oligonucleotide is an oligomer of 6, 8, 10, 12, 20, 30 or up to 100 nucleotides. In some embodiments, an oligonucleotide is an oligomer of at least 6, 8, 10, 12, 20, 30, 40, 50, 60, 70, 80, 90, 100, 150, 200, 300, 400, or 500 nucleotides. A “polynucleotide” or “oligonucleotide” may comprise DNA, RNA, PNA or a polymer of nucleotides linked by phosphodiester and/or any alternate bonds.

The phrases “percent homology,” “% homology,” “percent identity,” or “% identity” refer to the percentage of sequence similarity found in a comparison of two or more amino acid or nucleic acid sequences. Percent identity can be determined electronically, e.g., by using the MEGALIGN program (LASERGENE software package, DNASTAR). The MEGALIGN program can create alignments between two or more sequences according to different methods, e.g., the Clustal Method. (Higgins, D. G. and P. M. Sharp (1988) Gene 73:237-244.) The Clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Percent identity between nucleic acid sequences can also be calculated by the Clustal Method, or by other methods known in the art, such as the Jotun Hein Method. (See, e.g., Hein, J. (1990) Methods Enzymol. 183:626-645.) Identity between sequences can also be determined by other methods known in the art, e.g., by varying hybridization conditions.

By “pharmaceutically acceptable”, it is meant the carrier, diluent or excipient must be compatible with the other ingredients of the formulation and not deleterious to the recipient thereof.

As used herein, a polynucleotide “derived from” a designated sequence refers to a polynucleotide sequence which is comprised of a sequence of approximately at least about 6 nucleotides, preferably at least about 8 nucleotides, more preferably at least about 10-12 nucleotides, and even more preferably at least about 15-20 nucleotides corresponding to a region of the designated nucleotide sequence. “Corresponding” means homologous to or complementary to the designated sequence. Preferably, the sequence of the region from which the polynucleotide is derived is homologous to or complementary to a sequence that is unique to a cancer associated gene.

As used herein, the term “sample” refers to composition that is being tested or treated with a reagent, such as but not limited to a therapeutic, drug, or candidate agent. Samples may be obtained from subjects. In some embodiments, the sample may be blood, plasma, serum, or any combination thereof. A sample may be derived from blood, plasma, serum, or any combination thereof. Other typical samples include, but are not limited to, any bodily fluid obtained from a mammalian subject, tissue biopsy, sputum, lymphatic fluid, blood cells (e.g., peripheral blood mononuclear cells), tissue or fine needle biopsy samples, urine, peritoneal fluid, colostrums, breast milk, fetal fluid, fecal material, tears, pleural fluid, or cells therefrom. The sample may be processed in some manner before being used in a method described herein, for example a particular component to be analyzed or tested according to any of the methods described infra. One or more molecules may be isolated from a sample.

The terms “specific binding,” “specifically binds,” and the like, refer to instances where two or more molecules form a complex that is measurable under physiologic or assay conditions and is selective. An antibody or antigen binding protein or other molecule is said to “specifically bind” to a protein, antigen, or epitope if, under appropriately selected conditions, such binding is not substantially inhibited, while at the same time non-specific binding is inhibited. Specific binding is characterized by a high affinity and is selective for the compound, protein, epitope, or antigen. Nonspecific binding usually has a low affinity.

As used herein, a “recombinant protein” is a protein made using recombinant techniques, for example, but not limited to, through the expression of a recombinant nucleic acid as depicted above. A recombinant protein may be distinguished from naturally occurring protein by at least one or more characteristics. For example, the protein may be isolated or purified away from some or all of the proteins and compounds with which it is normally associated in its wild type host, and thus may be substantially pure. For example, an isolated protein is unaccompanied by at least some of the material with which it is normally associated in its natural state, preferably constituting at least about 0.5%, more preferably at least about 5% by weight of the total protein in a given sample. A substantially pure protein comprises about 50-75%, about 80%, or about 90%. In some embodiments, a substantially pure protein comprises about 80-99%, 85-99%, 90-99%, 95-99%, or 97-99% by weight of the total protein. A recombinant protein can also include the production of a cancer associated protein from one organism (e.g. human) in a different organism (e.g. yeast, E. coli, or the like) or host cell. Alternatively, the protein may be made at a significantly higher concentration than is normally seen, through the use of an inducible promoter or high expression promoter, such that the protein is made at increased concentration levels. Alternatively, the protein may be in a form not normally found in nature, as in the addition of an epitope tag or amino acid substitutions, insertions and deletions, as discussed herein.

The term “support” refers to conventional supports such as beads, particles, dipsticks, fibers, filters, membranes, and silane or silicate supports such as glass slides.

As used herein, the term “tag,” “sequence tag” or “primer tag sequence” refers to an oligonucleotide with specific nucleic acid sequence that serves to identify a batch of polynucleotides bearing such tags therein. Polynucleotides from the same biological source are covalently tagged with a specific sequence tag so that in subsequent analysis the polynucleotide can be identified according to its source of origin. The sequence tags also serve as primers for nucleic acid amplification reactions.

As used herein, the term “therapeutic” or “therapeutic agent” means an agent that can be used to treat, combat, ameliorate, prevent or improve an unwanted condition or disease of a patient. In part, embodiments of the present disclosure are directed to the treatment of cancer or the decrease in proliferation of cells. In some embodiments, the term “therapeutic” or “therapeutic agent” may refer to any molecule that associates with or affects the target marker, its expression or its function. In various embodiments, such therapeutics may include molecules such as, for example, a therapeutic cell, a therapeutic peptide, a therapeutic gene, a therapeutic compound, or the like, that associates with or affects the target marker, its expression or its function.

A “therapeutically effective amount” or “effective amount” of a composition is a predetermined amount calculated to achieve the desired effect, i.e., to inhibit, block, or reverse the activation, migration, or proliferation of cells. In some embodiments, the effective amount is a prophylactic amount. In some embodiments, the effective amount is an amount used to medically treat the disease or condition. The specific dose of a composition administered according to this invention to obtain therapeutic and/or prophylactic effects will, of course, be determined by the particular circumstances surrounding the case, including, for example, the composition administered, the route of administration, and the condition being treated. It will be understood that the effective amount administered will be determined by the physician in the light of the relevant circumstances including the condition to be treated, the choice of composition to be administered, and the chosen route of administration. A therapeutically effective amount of composition of this invention is typically an amount such that when it is administered in a physiologically tolerable excipient composition, it is sufficient to achieve an effective systemic concentration or local concentration in the targeted tissue.

The terms “treat,” “treated,” or “treating” as used herein can refer to both therapeutic treatment or prophylactic or preventative measures, wherein the object is to prevent or slow down (lessen) an undesired physiological condition, disorder or disease, or to obtain beneficial or desired clinical results. In some embodiments, the term may refer to both treating and preventing. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

The term “tissue” as used herein, refers to any aggregation of similarly specialized cells that are united in the performance of a particular function.

As used herein, the term “optional” or “optionally” refers to embodiments where the subsequently described structure, event or circumstance may or may not occur, and that the description includes instances where the event occurs and instances where it does not.

### Cancers

Various embodiments described herein provide methods, compositions and kits for the treatment, diagnosis, prognosis, visualization and detection of cancer. The embodiments described herein relate to any cancer including apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, merkel cell, mucinous, non-small cell lung, oat cell, papillary, scirrhous, bronchiolar, bronchogenic, squamous cell, and transitional-cell), histiocytic disorders, leukemia (e.g., b-cell, mixed-cell, null-cell, T-cell, T-cell chronic, HTLV-II-associated, lyphocytic acute, lymphocytic chronic, mast-cell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, osteosarcoma, rhabdomyosarcoma, sarcoma (e.g., Ewing's, experimental, Kaposi's, and mast-cell), neoplasms of the bone, breast, digestive system, colorectal, liver, pancreatic, pituitary, testicular, orbital, head and neck, central nervous system, acoustic, pelvic, respiratory tract, and urogenital trect, neurofibromatosis, cervix dysplasia, or a combination thereof. The methods disclosed herein may also be used for diagnosis and treatment of other conditions in which cells have become immortalized. In some embodiments, the cancer may be selected from Breast Tumor Infiltrating Ductal Carcinoma, Breast Tumor Lobular carcinoma, Adenocarcinoma of colon, Cervix Tumor Squamous cell carcinoma, Cervix Tumor Adenocarcinoma, Ovary Tumor Carcinoma, Ovary Tumor Serous Cystadenocarcinoma, Lung Carcinoma of lung squamous cell, Lung Adenocarcinoma of lung, Lung Carcinoma of lung large cell, Lung Tumor Non-Small Cell Carcinoma Adenocarcinoma, Pleura Mesothelioma, Esophagus Tumor Squamous cell carcinoma, Urinary bladder Carcinoma of bladder transitional cell, Pancreas Adenocarcinoma of pancreas ductal, Pancreas Gland Tumor Neuroendocrine carcinoma large cell, Testis Seminoma of testis, Bile duct Cholangiocarcinoma of bile duct, Stomach Tumor Adenocarcinoma, Stomach Tumor Adenocarcinoma Intestinal Type, Breast primary tumor, Colon primary tumor, MP Lung primary tumor, Rectum primary tumor, Breast Adenocarcinoma of breast metastatic, Colon Adenocarcinoma of colon metastatic, Ovary Adenocarcinoma of ovary serous metastatic, Kidney Carcinoma of kidney renal cell metastatic, Gastroesophageal junction Adenocarcinoma of gastroesophageal junction metastatic, Neck Carcinoma of neck squamous cell metastatic, Thyroid gland Carcinoma of thyroid papillary metastatic, Urinary bladder Carcinoma of bladder small cell metastatic, Prostate Adenocarcinoma of prostate metastatic, MP2 Colon metastatic tumor, Rectum metastatic tumor, Soft Tissue Tumor Metastatic neoplasm adenocarcinoma Serous cystadenocarcinoma, Liver Tumor Metastatic Neoplasm Adenocarcinoma, Connective Tissue Tumor Giant cell tumor of soft parts malignant, Cartilage Chondrosarcoma, Bone Osteosarcoma metastatic, Smooth muscle Sarcoma metastatic consistent with leiomyosarcoma primary, and Endometrium Endometrial stromal sarcomametastatic. Other cancers include neoplasms of the bone, breast, digestive system, colorectal, liver, pancreatic, pituitary, testicular, orbital, head and neck, central nervous system, acoustic, pelvic, respiratory tract, and urogenital trect, neurofibromatosis, cervix dysplasia.

Cancers classified by site include, but are not limited to, cancer of the oral cavity and pharynx (lip, tongue, salivary gland, floor of mouth, gum and other mouth, nasopharynx, tonsil, oropharynx, hypopharynx, other oral/pharynx); cancers of the digestive system (esophagus; stomach; small intestine; colon and rectum; anus, anal canal, and anorectum; liver; intrahepatic bile duct; gallbladder; other biliary; pancreas; retroperitoneum; peritoneum, omentum, and mesentery; other digestive); cancers of the respiratory system (nasal cavity, middle ear, and sinuses; larynx; lung and bronchus; pleura; trachea, mediastinum, and other respiratory); cancers of the mesothelioma; bones and joints; and soft tissue, including heart; skin cancers, including melanomas and other non-epithelial skin cancers; Kaposi's sarcoma and breast cancer; cancer of the female genital system (cervix uteri; corpus uteri; uterus, nos; ovary; vagina; vulva; and other female genital); cancers of the male genital system (prostate gland; testis; penis; and other male genital); cancers of the urinary system (urinary bladder; kidney and renal pelvis; ureter; and other urinary); cancers of the eye and orbit; cancers of the brain and nervous system (brain; and other nervous system); cancers of the endocrine system (thyroid gland and other endocrine, including thymus); lymphomas (Hodgkin's disease and non-Hodgkin's lymphoma), multiple myeloma, and leukemias (lymphocytic leukemia; myeloid leukemia; monocytic leukemia; and other leukemias).

In some embodiments relating to the diagnosis, prognosis, detection, treatment of cancer disclosed herein, the cancer may be chosen from breast, bladder, lung, colon, pancreatic, kidney and colon cancer. In some embodiments relating to compositions of matter, such as isolated proteins peptides, nucleic acids, kits and components of kits the cancer may be chosen from breast, bladder, lung, colon, pancreatic, kidney and colon cancer.

### Cancer Associated Sequences

In some embodiments, the present disclosure provides for nucleic acid and protein sequences that are associated with cancer, herein termed “cancer associated” or “CA” sequences. The nucleic acids and/or proteins may be encoded for by any of the genes provided below. The cancer associated sequences may be associated with any of the cancers disclosed infra. Thus the cancer associated sequences may be expressed in cancer cells found in tumors of any of the cancers disclosed infra. In some embodiments the cancer associate sequence is expressed at higher levels in a cancer cell found in a tumor, compared to a non-cancer cell, such as a non-cancer cell of the same tissue type found in the cancer.

In some embodiments, the term “cancer associated sequences” may indicate that the nucleotide or protein sequences are differentially expressed, activated, inactivated or altered in cancers as compared to normal tissue. Cancer associated sequences may include those that are up-regulated (i.e. expressed at a higher level), as well as those that are down-regulated (i.e. expressed at a lower level), in cancers. Cancer associated sequences can also include sequences that have been altered (i.e., translocations, truncated sequences or sequences with substitutions, deletions or insertions, including, but not limited to, point mutations) and show either the same expression profile or an altered profile. In some embodiments, the cancer associated sequences are from humans; however, as will be appreciated by those in the art, cancer associated sequences from other organisms, including any subject, may be useful in animal models of disease and drug evaluation; thus, other cancer associated sequences may be useful such as, without limitation, sequences from vertebrates, including mammals, including rodents (rats, mice, hamsters, guinea pigs, etc.), primates, and farm animals (including sheep, goats, pigs, cows, horses, etc.). Cancer associated sequences from other organisms may be obtained using the techniques outlined herein.

Cancer associated sequences include nucleic acid sequences and fragments thereof encoding one or more markers associated with a diagnosis, prognosis and treatment of cancer. The sequences can be DNA sequences, included isolated DNA sequences and RNA such as mRNA sequences including isolated RNA sequences. Cancer associated sequences also include proteins and peptide fragments encoded for by DNA from a cancer associated sequence such as DNA sequence encoding one or more of the following genes: Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5. Any one or combination of 2 or more of the foregoing cancer associated sequences may be used in any of the embodiments disclosed herein, including methods of diagnosing, detecting, visualizing and treating cancer, as well as compositions and kits related to the treatment, detection, visualization and diagnosis of cancer as described infra.

In some embodiments, cancer associated sequences may include both nucleic acid and amino acid sequences. In some embodiments, the cancer associated sequences may include sequences having at least about 60% homology with the disclosed sequences. In some embodiments, the cancer associated sequences may have at least about 65%, about 70%, about 75%, about 80%, about 85%, about 90%, about 95%, about 97%, about 99%, about 99.8% homology with the disclosed sequences. In some embodiments, the cancer associated sequences may be “mutant nucleic acids”. As used herein, “mutant nucleic acids” refers to deletion mutants, insertions, point mutations, substitutions, translocations.

In some embodiments, the cancer associated sequences are nucleic acids. As will be appreciated by those skilled in the art and is described herein, cancer associated sequences of embodiments herein may be useful in a variety of applications including diagnostic applications to detect nucleic acids or their expression levels in a subject, therapeutic applications or a combination thereof. Further, the cancer associated sequences of embodiments herein may be used in screening applications; for example, generation of biochips comprising nucleic acid probes to the cancer associated sequences.

In some embodiments, the cancer associated sequences may be recombinant nucleic acids. By the term “recombinant nucleic acid” herein refers to nucleic acid molecules, originally formed in vitro, in general, by the manipulation of nucleic acid by polymerases and endonucleases, in a form not normally found in nature. Thus a recombinant nucleic acid may also be an isolated nucleic acid, in a linear form, or cloned in a vector formed in vitro by ligating DNA molecules that are not normally joined, are both considered recombinant for the purposes of this invention. It is understood that once a recombinant nucleic acid is made and reintroduced into a host cell or organism, it can replicate using the in vivo cellular machinery of the host cell rather than in vitro manipulations; however, such nucleic acids, once produced recombinantly, although subsequently replicated in vivo, are still considered recombinant or isolated for the purposes of the invention. As used herein, a “polynucleotide” or “nucleic acid” is a polymeric form of nucleotides of any length, either ribonucleotides or deoxyribonucleotides. This term includes double- and single-stranded DNA and RNA. It also includes known types of modifications, for example, labels which are known in the art, methylation, “caps”, substitution of one or more of the naturally occurring nucleotides with an analog, internucleotide modifications-such as, for example, those with uncharged linkages (e.g., phosphorothioates, phosphorodithioates, etc.), those containing pendant moieties, such as, for example proteins (including e.g., nucleases, toxins, antibodies, signal peptides, poly-L-lysine, etc.), those with intercalators (e.g., acridine, psoralen, etc.), those containing chelators (e.g., metals, radioactive metals, etc.), those containing alkylators, those with modified linkages (e.g., alpha anomeric nucleic acids, etc.), as well as unmodified forms of the polynucleotide.

A nucleic acid of the present disclosure may include phosphodiester bonds, although in some cases, as outlined below (for example, in antisense applications or when a nucleic acid is a candidate drug agent), nucleic acid analogs may have alternate backbones, comprising, for example, phosphoramidate (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within one definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done for a variety of reasons, for example to increase the stability and half-life of such molecules in physiological environments for use in anti-sense applications or as probes on a biochip.

As will be appreciated by those skilled in the art, such nucleic acid analogs may be used in some embodiments of the present disclosure. In addition, mixtures of naturally occurring nucleic acids and analogs can be made; alternatively, mixtures of different nucleic acid analogs, and mixtures of naturally occurring nucleic acids and analogs may be made.

In some embodiments, the nucleic acids may be single stranded or double stranded or may contain portions of both double stranded or single stranded sequence. As will be appreciated by those skilled in the art, the depiction of a single strand also defines the sequence of the other strand; thus the sequences described herein also includes the complement of the sequence. The nucleic acid may be DNA, both genomic and cDNA, RNA, or a hybrid, where the nucleic acid contains any combination of deoxyribo- and ribo-nucleotides, and any combination of bases, including uracil, adenine, thymine, cytosine, guanine, inosine, xanthine, hypoxanthine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occurring analog structures. Thus, for example, the subject units of a peptide nucleic acid, each containing a base, are referred to herein as a nucleoside.

In some embodiments, an isolated nucleic acid comprises at least 10, 12, 15, 20 or 30 contiguous nucleotides of a sequence selected from the group consisting of the cancer associated polynucleotide sequences disclosed in Table 1.

In some embodiments, the polynucleotide, or its complement or a fragment thereof, further comprises a detectable label, is attached to a solid support, is prepared at least in part by chemical synthesis, is an antisense fragment, is single stranded, is double stranded or comprises a microarray.

In some embodiments, the invention provides an isolated polypeptide, encoded within an open reading frame of a cancer associated sequence selected from the polynucleotide sequences shown in Table 1, or its complement. In some embodiments, the invention provides an isolated polypeptide, wherein said polypeptide comprises the amino acid sequence encoded by a polynucleotide selected from the group consisting of the sequences disclosed in Table 1. In some embodiments, the invention provides an isolated polypeptide, wherein said polypeptide comprises the amino acid sequence encoded by a cancer associated polypeptide.

In some embodiments, the invention further provides an isolated polypeptide, comprising the amino acid sequence of an epitope of the amino acid sequence of a cancer associated polypeptide, wherein the polypeptide or fragment thereof may be attached to a solid support. In some embodiments the invention provides an isolated antibody (monoclonal or polyclonal) or antigen binding fragment thereof, that binds to such a polypeptide. The isolated antibody or antigen binding fragment thereof may be attached to a solid support, or further comprises a detectable label.

Some embodiments herein are directed to one or more sequences associated with cancer, including any cancer disclosed infra. The sequences disclosed herein may also be used for diagnosis and treatment of other conditions in which cells have become immortalized. The use of microarray analysis of gene expression allows the identification of host sequences associated with cancer. These sequences may then be used in a number of different ways, including diagnosis, prognosis, screening for modulators (including both agonists and antagonists), antibody generation (for immunotherapy and imaging), etc. However, as will be appreciated by those skilled in the art, sequences that are identified in one type of cancer may have a strong likelihood of being involved in other types of cancers as well. Thus, while the sequences outlined herein may be initially identified as correlated with one or more types of cancers, they may also be found in other types of cancers as well.

Some embodiments described herein may be directed to the use of cancer associated sequences for diagnosis, prognosis, visualization and treatment of cancer. Any of the cancer associated sequences disclosed herein may be used. The cancer types include any of the cancers disclosed infra. The markers disclosed herein may also be used for diagnosis and treatment of other conditions in which cells have become immortalized.

### Methods of Diagnosing and Detecting Cancer

In some embodiments, a method of diagnosing and/or detecting cancer in a sample may comprise detecting a level of the cancer associated protein in a sample. In some embodiments, a method of screening for cancer may comprise detecting a level of the cancer associated protein. In some embodiments, the cancer associated protein is encoded by a nucleotide sequence selected from the sequences disclosed in Table 1, a fraction thereof or a complementary sequence thereof or a cancer associated sequence disclosed infra. In some embodiments, technologies such as ELISA, as well as other detection methods may be used.

Any technique known in the art may be used to assay a sample for presence of cancer cells. In some embodiments, detecting a level of a cancer associated sequence may comprise techniques such as, but not limited to, PCR, mass spectroscopy, microarray or other detection techniques described herein. Other suitable techniques include the gel electrophoresis, gel shift assays, nuclear run on assays, ELISAs radio immuno-assays, flow cytometric assays, microscopy, such as fluorescent microscopy, affinity chromatography, immune-precipitation, branched RNA and the like. Information relating to expression of the receptor can also be useful in determining therapies aimed at up or down-regulating the cancer associated sequence's signaling using agonists or antagonists.

In some embodiments cancer can be detected in a sample by isolating a nucleic acid from the sample. The nucleic acid may be an RNA molecule such as an mRNA molecule. The RNA molecule can be transcribed into cDNA. The cDNA can be analyzed by gel electrophoresis. The cDNA can be transferred from the gel onto a support, such as membrane. The cDNA can be hybridized with a probe that specifically binds to it. The probe can be labeled with a detectable substance. The signal obtained from the detectable substance can be measured and the amount of cancer associated sequence present in the sample may be determined, e.g. by comparing the signal obtained with the signal obtained from a known quantity of the cancer associated sequence. Alternatively, the cDNA molecule can be amplified using PCR before gel electrophoresis. In another embodiment rtPCR or qPCR may be used to analyze and quantitate the amount of cancer associated sequence in sample.

In other embodiments a protein encoded for by a cancer associated sequence can be isolated from a sample and contacted with an antibody that specifically binds to the protein. The antibody can be label with a detectable substance. By measuring the signal from the detectable substance the amount of protein encoded for by the cancer associated sequence can be determined, e.g. by comparing the signal obtained using the same antibody on a known quantity of the protein.

In some embodiments, a subject can be diagnosed with cancer by detecting the presence of a cancer associated sequence selected from the sequences disclosed in Table 1. In some embodiments, a method of diagnosing a subject with cancer comprises detecting the presence of a cancer associated sequence selected from the sequences disclosed in Table 1, wherein the presence of the cancer associated sequence indicates that the subject has cancer. In some embodiments, the method comprises detecting the presence or absence of a cancer associated sequence selected from the sequences disclosed in Table 1, wherein the absence of the cancer associated sequence indicates that absence of cancer. In some embodiments, the method further comprises treating the subject diagnosed with cancer with an antibody that binds to a cancer associated sequence selected from the sequences disclosed in Table 1 and inhibits the growth or progression of the cancer. As discussed, cancer may be detected in any type of sample, including, but not limited to, serum, blood, tumor and the like. The sample may be any type of sample as it is described herein.

In some embodiments, the method of diagnosing a subject with cancer comprises obtaining a sample and detecting the presence of a cancer associated sequence selected from sequences disclosed in Table 1 wherein the presence of the cancer associated sequence indicates the subject has cancer. In some embodiments, detecting the presence of a cancer associated sequence selected from sequences disclosed in Table 1 comprises contacting the sample with an antibody or other type of capture reagent that specifically binds to the cancer associated sequence's protein and detecting the presence or absence of the binding to the cancer associated sequence's protein in the sample. An example of an assay that can be used includes but is not limited to, an ELISA.

In some embodiments, the present disclosure provides a method of diagnosing cancer, or a neoplastic condition in a subject, the method comprising obtaining a cancer associated sequence gene expression result of a cancer associated sequence selected from sequences disclosed in Table 1 from a sample derived from a subject; and diagnosing cancer or a neoplastic condition in the subject based on the cancer associated sequence gene expression result, wherein the subject is diagnosed as having cancer or a neoplastic condition if the cancer associated sequence is overexpressed.

In some embodiments, the subject is diagnosed as not having cancer, cancer, or a neoplastic condition if the cancer associated sequence is not overexpressed. In some embodiments of the methods described herein and throughout, the cancer that is diagnosed based upon a cancer associated sequence gene expression result or the absence or presence of a cancer associated sequence or protein. Any cancer associated sequence disclosed infra. may be used.

In some embodiments, a method of diagnosing a subject with cancer comprises obtaining a sample and detecting the presence of a cancer associated sequence selected from a sequence disclosed in Table 1 wherein the presence of the cancer associated sequence indicates the subject has cancer. In some embodiments, detecting the presence of a cancer associated sequence selected from a sequence disclosed in Table 1 comprises contacting the sample with an antibody or other type of capture reagent that specifically binds to the cancer associated sequence's protein and detecting the presence or absence of the binding to the cancer associated sequence's protein in the sample.

Some embodiments herein describe method of diagnosing cancer or a neoplastic condition comprising administering an antibody against the cancer associated sequence to a subject. In some embodiments, the antibody may be monoclonal or polyclonal. In some embodiments, the antibody may be humanized or recombinant. In some embodiments, the antibody may neutralize biological activity of the cancer associated sequence by binding to and/or interfering with the cancer associated sequence's receptor. In some embodiments, administering the antibody may be to a biological fluid or tissue, such as, without limitation, blood, urine, serum, tumor tissue, or the like. Some embodiments herein may be directed to a method of screening for cancer comprising detecting the presence of the cancer associated sequence in a biological sample. In some embodiments, the biological sample may be any biological fluid or tissue from a subject, such as, without limitation, blood, urine, serum, tumor tissue, or the like.

In some embodiments, the present disclosure provides methods of diagnosing cancer or a neoplastic condition in a subject, the method comprising obtaining a gene expression result of a cancer associated sequence as disclosed infra.

In some embodiments the invention provides a method of detecting cancer in a sample comprising analyzing the sample for the expression level of one or more of the genes chosen from GNGT1, C12orf56, COL10A1, SLC35D3, snaR-A, SBK1, DSCR8, CELSR3 or a complement thereof. An elevated level (compared to a non-cancerous sample) of expression of one or more of these genes indicates cancer cells are present in the sample.

Some embodiments are directed to a biochip comprising a nucleic acid segment which encodes a cancer associated protein. In some embodiments, a biochip comprises a nucleic acid molecule which encodes at least a portion of a cancer associated protein. In some embodiments, the cancer associated protein is encoded by a sequence selected from a sequence disclosed in Table 1, homologs thereof, combinations thereof, or a fragment thereof. In some embodiments, the nucleic acid molecule specifically hybridizes with a nucleic acid sequence selected from a sequence disclosed in Table 1. In some embodiments, the biochip comprises a first and second nucleic molecule wherein the first nucleic acid molecule specifically hybridizes with a first sequence selected from a sequence disclosed in Table 1 and the second nucleic acid molecule specifically hybridizes with a second sequence selected from a sequence disclosed in Table 1, wherein the first and second sequences are not the same sequence. In some embodiments, the present invention provides methods of detecting or diagnosing cancer comprising detecting the expression of a nucleic acid sequence selected from a sequence disclosed in Table 1, wherein a sample is contacted with a biochip comprising a sequence selected from a sequence disclosed in Table 1, homologs thereof, combinations thereof, or a fragment thereof.

In some embodiments, the invention provides a method for detecting a cancer associated sequence with the expression of a polypeptide in a test sample, comprising detecting a level of expression of at least one polypeptide such as, without limitation, a cancer associated protein, or a fragment thereof. In some embodiments, the method comprises comparing the level of expression of the polypeptide in the test sample with a level of expression of polypeptide in a normal sample, wherein an altered level of expression of the polypeptide in the test sample relative to the level of polypeptide expression in the normal sample is indicative of the presence of cancer in the test sample. In some embodiments, the polypeptide expression is compared to a cancer sample, wherein the level of expression is at least the same as the cancer is indicative of the presence of cancer in the test sample. In some embodiments, the sample is a cell sample.

In some embodiments, the invention provides a method for detecting cancer by detecting the presence of an antibody in a test serum sample. In some embodiments, the antibody recognizes a polypeptide or an epitope thereof disclosed herein. In some embodiments, the antibody recognizes a polypeptide or epitope thereof encoded by a nucleic acid sequence disclosed herein. In some embodiments, the method comprises detecting a level of an antibody against an antigenic polypeptide such as, without limitation, a cancer associated protein, or an antigenic fragment thereof. In some embodiments, the method comprises comparing the level of the antibody in the test sample with a level of the antibody in the control sample, wherein an altered level of antibody in said test sample relative to the level of antibody in the control sample is indicative of the presence of cancer in the test sample. In some embodiments, the control sample is a sample derived from a normal cell or non-cancerous sample. In some embodiments, the control is derived from a cancer sample, and, therefore, in some embodiments, the method comprises comparing the levels of binding and/or the amount of antibody in the sample, wherein when the levels or amount are the same as the cancer control sample is indicative of the presence of cancer in the test sample.

In some embodiments, a method for diagnosing cancer or a neoplastic condition comprises a) determining the expression of one or more genes comprising a nucleic acid sequence selected from the group consisting of the human genomic and mRNA sequences described in Table 1, in a first sample type (e.g. tissue) of a first individual; and b) comparing said expression of said gene(s) from a second normal sample type from said first individual or a second unaffected individual; wherein a difference in said expression indicates that the first individual has cancer. In some embodiments, the expression is increased as compared to the normal sample. In some embodiments, the expression is decreased as compared to the normal sample.

In some embodiments, the invention also provides a method for detecting presence or absence of cancer cells in a sample from a subject. In some embodiments, the method comprises contacting one or more cells from the subject with an antibody as described herein. In some embodiments, the method comprises detecting a complex of a cancer associated protein and the antibody, wherein detection of the complex indicates with the presence of cancer cells in the subject. In some embodiments the invention provides a method for inhibiting growth of cancer cells in a subject. In some embodiments, the method comprises administering to the subject an effective amount of a pharmaceutical composition as described herein. In some embodiments the invention provides a method for delivering a therapeutic agent to cancer cells in a subject, the method comprising: administering to the subject an effective amount of a pharmaceutical composition according to according to the invention.

In some embodiments, the present disclosure provides methods of diagnosing cancer or a neoplastic condition in a subject, the method comprising: a) determining the expression of one or more genes or gene products or homologs thereof; and b) comparing said expression of the one or more nucleic acid sequences from a second normal sample from said first subject or a second unaffected subject, wherein a difference in said expression indicates that the first subject has cancer.

In some embodiments, the present disclosure provides methods of detecting cancer in a test sample, comprising: (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample.

### Screening for Cancer Therapeutics and Cancer Inhibitors

In some embodiments, a method of identifying an anti-cancer agent is provided, wherein the method comprises contacting a candidate agent to a sample; and determining the cancer associated sequence's activity in the sample. In some embodiments, the candidate agent is identified as an anti-cancer agent if the cancer associated sequence's activity is reduced in the sample after the contacting. In some embodiments, the candidate agent is a candidate antibody. In some embodiments, the method comprises contacting a candidate antibody that binds to the cancer associated sequence with a sample, and assaying for the cancer associated sequence's activity, wherein the candidate antibody is identified as an anti-cancer agent if the cancer associated sequence activity is reduced in the sample after the contacting. A cancer associated sequence's activity can be any activity of the cancer associated sequence.

In some embodiments, the present disclosure provides methods of identifying an anti cancer (e.g. cancer) agent, the method comprising contacting a candidate agent to a cell sample; and determining activity of one or more cancer associated sequences disclosed infra. Th activity can be measured by any method known in the art.

In some embodiments, a method of screening drug candidates includes comparing the level of expression of the cancer-associated sequence in the absence of the drug candidate to the level of expression in the presence of the drug candidate.

Some embodiments are directed to a method of screening for a therapeutic agent capable of binding to a cancer-associated sequence (nucleic acid or protein), the method comprising combining the cancer-associated sequence and a candidate therapeutic agent, and determining the binding of the candidate agent to the cancer-associated sequence.

Further provided herein is a method for screening for a therapeutic agent capable of modulating the activity of a cancer-associated sequence. In some embodiments, the method comprises combining the cancer-associated sequence and a candidate therapeutic agent, and determining the effect of the candidate agent on the bioactivity of the cancer-associated sequence. An agent that modulates the bioactivity of a cancer associated sequence may be used as a therapeutic agent capable of modulating the activity of a cancer-associated sequence.

A method of screening for anticancer activity, the method comprising: (a) contacting a cell that expresses a cancer associated gene which transcribes a cancer associated sequence selected from a sequence disclosed in Table 1, homologs thereof, combinations thereof, or fragments thereof with an anticancer drug candidate; (b) detecting an effect of the anticancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has anticancer activity.

In some embodiments, a method of evaluating the effect of a candidate cancer drug may comprise administering the drug to a patient and removing a cell sample from the patient. The expression profile of the cell is then determined. In some embodiments, the method may further comprise comparing the expression profile of the patient to an expression profile of a healthy individual. In some embodiments, the expression profile comprises measuring the expression of one or more or any combination thereof of the sequences disclosed herein. In some embodiments, where the expression profile of one or more or any combination thereof of the sequences disclosed herein is modified (increased or decreased) the candidate cancer drug is said to be effective.

In some embodiments, the invention provides a method of screening for anticancer activity comprising: (a) providing a cell that expresses a cancer associated gene that encodes a nucleic acid sequence selected from the group consisting of the cancer associated sequences shown in Table 1, or fragment thereof, (b) contacting the cell, which can be derived from a cancer cell with an anticancer drug candidate; (c) monitoring an effect of the anticancer drug candidate on an expression of the cancer associated sequence in the cell sample, and optionally (d) comparing the level of expression in the absence of said drug candidate to the level of expression in the presence of the drug candidate. The drug candidate may be an inhibitor of transcription, a G-protein coupled receptor antagonist, a growth factor antagonist, a serine-threonine kinase antagonist, a tyrosine kinase antagonist. In some embodiments, where the candidate modulates the expression of the cancer associated sequence the candidate is said to have anticancer activity. In some embodiments, the anticancer activity is determined by measuring cell growth. In some embodiments, the candidate inhibits or retards cell growth and is said to have anticancer activity. In some embodiments, the candidate causes the cell to die, and thus, the candidate is said to have anticancer activity.

In some embodiments, the present invention provides a method of screening for activity against cancer. In some embodiments, the method comprises contacting a cell that overexpresses a cancer associated gene which is complementary to a cancer associated sequence selected from the sequences disclosed in Table 1, homologs thereof, combinations thereof, or fragments thereof with a cancer drug candidate. In some embodiments, the method comprises detecting an effect of the cancer drug candidate on an expression of the cancer associated polynucleotide in the cell or an effect on the cell's growth or viability. In some embodiments, the method comprises comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against a cancer cell that overexpresses a cancer associated gene, wherein said gene comprises a sequence that is a sequence selected from the sequences disclosed in Table 1, or complementary thereto, homologs thereof, combinations thereof, or fragments thereof. In some embodiments, the drug candidate is selected from a transcription inhibitor, a G-protein coupled receptor antagonist, a growth factor antagonist, a serine-threonine kinase antagonist, or a tyrosine kinase antagonist.

In some embodiments, the invention provides a method for screening for a therapeutic agent capable of modulating the activity of a cancer associated sequence, wherein said sequence can be encoded by a nucleic acid comprising a nucleic acid sequence selected from the group consisting of the polynucleotide sequences shown in Table 1, said method comprising: a) combining said cancer associated sequence and a candidate therapeutic agent; and b) determining the effect of the candidate agent on the bioactivity of said cancer associated sequence. In some embodiments, the therapeutic agent: affects the expression of the cancer associated sequence; affects the activity of the cancer associated sequence. In some embodiments, the cancer associated sequence is a cancer associated protein. In some embodiments, the cancer associated sequence is a cancer associated nucleic acid molecule.

### Methods for Identifying Cancer Markers

Some embodiments of the invention include methods of screening a sample for a cancer marker, e.g. a cancer associated sequence. Cells can be screened using any technique known in the art. For example microarrays can be used. Gene expression can be analyzed in cells from the sample. Comparisons between samples known to contain cancer cells and samples known to be free of cancer cells can be made. The samples containing the cancer cells and those free of cancer may be comprised of cells of the same tissue type.

Some embodiments of the invention are directed to methods of identifying novel target markers useful in the diagnosis and treatment of cancer wherein expression levels of mRNAs, miRNAs, proteins, or protein post translational modifications including but not limited to phosphorylation and sumoylation are compared between five categories of cell types: (1) immortal pluripotent stem cells (such as embryonic stem (“ES”) cells, induced pluripotent stem (“iPS”) cells, and germ-line cells such as embryonal carcinoma (“EC”) cells) or gonadal tissues; (2) ES, iPS, or EC-derived clonal embryonic progenitor (“EP”) cell lines, (3) nucleated blood cells including but not limited to CD34+ cells and CD133+ cells; (4) normal mortal somatic adult-derived tissues and cultured cells including: skin fibroblasts, vascular endothelial cells, normal non-lymphoid and non-cancerous tissues, and the like, and (5) malignant cancer cells including cultured cancer cell lines or human tumor tissue. mRNAs, miRNAs, or proteins that are generally expressed (or not expressed) in categories 1, 3, and 5, or categories 1 and 5 but not expressed (or expressed) in categories 2 and 4 are candidate targets for cancer diagnosis and therapy. Some embodiments herein are directed to human applications, non-human veterinary applications, or a combination thereof.

In some embodiments, a method of identifying a target marker comprises the steps of: 1) obtaining a molecular profile of the mRNAs, miRNAs, proteins, or protein modifications of immortal pluripotent stem cells (such as embryonic stem (“ES”) cells, induced pluripotent stem (“iPS”) cells, and germ-line cells such as embryonal carcinoma (“EC”) cells); 2) ES, iPS, or EC-derived clonal embryonic progenitor (“EP”) cell lines malignant cancer cells including cultured cancer cell lines or human tumor tissues, and comparing those molecules to those present in mortal somatic cell types such as cultured clonal human embryonic progenitors, cultured somatic cells from fetal or adult sources, or normal tissue counterparts to malignant cancer cells. Target markers that are shared between pluripotent stem cells such as hES cells and malignant cancer cells, but are not present in a majority of somatic cell types may be candidate diagnostic markers and therapeutic targets.

Cancer associated sequences of embodiments herein are disclosed, for example, in Table 1. These sequences were extracted from fold-change and filter analysis KC110729.5. Expression of these cancer associated sequences in normal and tumor tissues is disclosed in Table 2. Once expression was determined, the gene sequence results were further filtered by considering fold-change in cancer cell lines vs. normal tissue; general specificity; secreted or not, level of expression in cancer cell lines; and signal to noise ratio. The cancer associated polynucleotide sequences include a sequence disclosed in Table 1 or a homolog thereof. In some embodiments, the polynucleotide sequences may be mRNA sequences selected from a sequence disclosed in Table 1, a complement thereof or a homolog thereof. In some embodiments, the cancer associated sequences may be DNA sequences encoding the above mRNA or the cancer associated protein or cancer associated polypeptide expressed by the above mRNA or homologs thereof. In some embodiments, the cancer associated sequence may be a mutant nucleic acid of the above disclosed sequences. In some embodiments, the homolog may have at least about 60%, at least about 65%, at least about 70%, at least about 75%, at least about 80%, at least about 85%, at least about 90%, at least about 95%, at least about 97%, at least about 98%, at least about 99%, at least about 99.5% identity with the disclosed polypeptide sequence.

The disclosed methods for identifying diagnostic and/or detection markers of cancer and therapeutic targets for the same overlap in some but not all cases with telomerase activity. The disclosed methods may be used to screen for markers for any cancer, including without limitation: apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, merkel cell, mucinous, non-small cell lung, oat cell, papillary, scirrhous, bronchiolar, bronchogenic, squamous cell, and transitional-cell), histiocytic disorders, leukemia (e.g., b-cell, mixed-cell, null-cell, T-cell, T-cell chronic, HTLV-II-associated, lyphocytic acute, lymphocytic chronic, mast-cell, and myeloid), histiocytosis malignant, Hodgkin's disease, immunoproliferative small, non-Hodgkin's lymphoma, plasmacytoma, reticuloendotheliosis, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, craniopharyngioma, dysgerminoma, hamartoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hem angioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, osteosarcoma, rhabdomyosarcoma, sarcoma (e.g., Ewing's, experimental, Kaposi's, and mast-cell), neoplasms of the bone, breast, digestive system, colorectal, liver, pancreatic, pituitary, testicular, orbital, head and neck, central nervous system, acoustic, pelvic, respiratory tract, and urogenital trect, neurofibromatosis, and cervix dysplasia, and for treatment of other conditions in which cells have become immortalized.

The pattern of gene expression in a particular living cell may be characteristic of its current state. Nearly all differences in the state or type of a cell are reflected in the differences in RNA levels of one or more genes. Comparing expression patterns of uncharacterized genes may provide clues to their function. High throughput analysis of expression of hundreds or thousands of genes can help in (a) identification of complex genetic diseases, (b) analysis of differential gene expression over time, between tissues and disease states, and (c) drug discovery and toxicology studies. Increase or decrease in the levels of expression of certain genes correlate with cancer biology. For example, oncogenes are positive regulators of tumorigenesis, while tumor suppressor genes are negative regulators of tumorigenesis. (Marshall, Cell, 64: 313-326 (1991); Weinberg, Science, 254: 1138-1146 (1991)). Accordingly, some embodiments herein provide for polynucleotide and polypeptide sequences involved in cancer and, in particular, in oncogenesis.

Oncogenes are genes that can cause cancer. Carcinogenesis can occur by a wide variety of mechanisms, including infection of cells by viruses containing oncogenes, activation of protooncogenes in the host genome, and mutations of protooncogenes and tumor suppressor genes. Carcinogenesis is fundamentally driven by somatic cell evolution (i.e. mutation and natural selection of variants with progressive loss of growth control). The genes that serve as targets for these somatic mutations are classified as either protooncogenes or tumor suppressor genes, depending on whether their mutant phenotypes are dominant or recessive, respectively.

It will be appreciated that there are various methods of obtaining expression data and uses of the expression data. For example, the expression data that can be used to detect or diagnose a subject with cancer can be obtained experimentally. In some embodiments, obtaining the expression data comprises obtaining the sample and processing the sample to experimentally determine the expression data. The expression data can comprise expression data for one or more of the cancer associated sequences described herein. The expression data can be experimentally determined by, for example, using a microarray or quantitative amplification method such as, but not limited to, those described herein. In some embodiments, obtaining expression data associated with a sample comprises receiving the expression data from a third party that has processed the sample to experimentally determine the expression data.

Detecting a level of expression or similar steps that are described herein may be done experimentally or provided by a third-party as is described herein. Therefore, for example, “detecting a level of expression” may refer to experimentally measuring the data and/or having the data provided by another party who has processed a sample to determine and detect a level of expression data. In some embodiments, the expression data may be detected experimentally and provided by a third party.

The comparison of gene expression on an mRNA level using Illumina gene expression microarrays hybridized to RNA probe sequences (shown in Table 1) prepared from the diverse categories of cell types: 1) human embryonic stem (“ES”) cells, or gonadal tissues 2) ES, iPS, or EC-derived clonal embryonic progenitor (“EP”) cell lines, 3) nucleated blood cells including but not limited to CD34+ cells and CD133+ cells; 4) Normal mortal somatic adult-derived tissues and cultured cells including: skin fibroblasts, vascular endothelial cells, normal non-lymphoid and non-cancerous tissues, and the like, and 5) malignant cancer cells including cultured cancer cell lines or human tumor tissue and filters was performed to detect genes that are generally expressed (or not expressed) in categories 1, 3, and 5, or categories 1 and 5 but not expressed (or expressed) in categories 2 and 4. Therapies in these cancers based on this observation would be based on reducing the expression of the above referenced transcripts up-regulated in cancer, or otherwise reducing the expression of the gene products.

Gene Expression Assays: Measurement of the gene expression levels may be performed by any known methods in the art, including but not limited to quantitative PCR, or microarray gene expression analysis, bead array gene expression analysis and Northern analysis. The gene expression levels may be represented as relative expression normalized to the ADPRT (Accession number NM_{—}001618.2), GAPD (Accession number NM_{—}002046.2), or other housekeeping genes known in the art. In the case of microarrayed probes of mRNA expression, the gene expression data may also be normalized by a median of medians method. In this method, each array gives a different total intensity. Using the median value is a robust way of comparing cell lines (arrays) in an experiment. As an example, the median was found for each cell line and then the median of those medians became the value for normalization. The signal from the each cell line was made relative to each of the other cell lines.

RNA extraction: Cells of the present disclosure may be incubated with 0.05% trypsin and 0.5 mM EDTA, followed by collecting in DMEM (Gibco, Gaithersburg, Md.) with 0.5% BSA. Total RNA may be purified from cells using the RNeasy Mini kit (Qiagen, Hilden, Germany).

Isolation of total RNA and miRNA from human embryonic stem cells and differentiated progeny cells: Total RNA or samples enriched for small RNA species may be isolated from cell cultures that undergo serum starvation prior to harvesting RNA to approximate cellular growth arrest observed in many mature tissues. Cellular growth arrest may be performed by changing to medium containing 0.5% serum for 5 days, with one medium change 2-3 days after the first addition of low serum medium. RNA may be harvested according to the vendor's instructions for Qiagen RNEasy kits to isolate total RNA or Ambion mirVana kits to isolate RNA enriched for small RNA species. The RNA concentrations may be determined by spectrophotometry and RNA quality may be determined by denaturing agarose gel electrophoresis to visualize 28S and 18S RNA. Samples with clearly visible 28S and 18S bands without signs of degradation and at a ratio of approximately 2:1, 28S:18S may be used for subsequent miRNA analysis.

Assay for miRNA in samples isolated from human embryonic stem cells and differentiated progeny cells: The miRNAs may be quantitated using a Human Panel TaqMan MicroRNA Assay from Applied Biosystems, Inc. This is a two-step assay that uses stem-loop primers for reverse transcription (RT) followed by real-time TaqMan®. A total of 330 miRNA assays may be performed to quantitate the levels of miRNA in the H9 human embryonic stem cell line, a differentiated fibroblast cell line, and nine cell lines differentiated from human embryonic stem cells. The assay includes two steps, reverse transcription (RT) and quantitative PCR. Real-time PCR may be performed on an Applied Biosystems 7500 Real-Time PCR System. The copy number per cell may be estimated based on the standard curve of synthetic mir-16 miRNA and assuming a total RNA mass of approximately 15 pg/cell.

The reverse transcription reaction may be performed using 1× cDNA archiving buffer, 3.35 units MMLV reverse transcriptase, 5 mM each dNTP, 1.3 units AB RNase inhibitor, 2.5 nM 330-plex reverse primer (RP), 3 ng of cellular RNA in a final volume of 5 μl. The reverse transcription reaction may be performed on a BioRad or MJ thermocycler with a cycling profile of 20° C. for 30 sec; 42° C. for 30 sec; 50° C. for 1 sec, for 60 cycles followed by one cycle of 85° C. for 5 min.

Real-time PCR. Two microlitres of 1:400 diluted Pre-PCR product may be used for a 20 ul reaction. All reactions may be duplicated. Because the method is very robust, duplicate samples may be sufficient and accurate enough to obtain values for miRNA expression levels. TaqMan universal PCR master mix of ABI may be used according to manufacturer's suggestion. Briefly, 1× TaqMan Universal Master Mix (ABI), 1 uM Forward Primer, 1 uM Universal Reverse Primer and 0.2 uM TaqMan Probe may be used for each real-time PCR. The conditions used may be as follows: 95° C. for 10 min, followed by 40 cycles at 95° C. for 15 s, and 60° C. for 1 min. All the reactions may be run on ABI Prism 7000 Sequence Detection System.

Microarray hybridization and data processing. cDNA samples and cellular total RNA (5 μg in each of eight individual tubes) may be subjected to the One-Cycle Target Labeling procedure for biotin labeling by in vitro transcription (IVT) (Affymetrix, Santa Clara, Calif.) or using the Illumina Total Prep RNA Labelling kit. For analysis on Affymetix gene chips, the cRNA may be subsequently fragmented and hybridized to the Human Genome U133 Plus 2.0 Array (Affymetrix) according to the manufacturer's instructions. The microarray image data may be processed with the GeneChip Scanner 3000 (Affymetrix) to generate CEL data. The CEL data may be then subjected to analysis with dChip software, which has the advantage of normalizing and processing multiple datasets simultaneously. Data obtained from the eight nonamplified controls from cells, from the eight independently amplified samples from the diluted cellular RNA, and from the amplified cDNA samples from 20 single cells may be normalized separately within the respective groups, according to the program's default setting. The model based expression indices (MBEI) may be calculated using the PM/MM difference mode with log-2 transformation of signal intensity and truncation of low values to zero. The absolute calls (Present, Marginal and Absent) may be calculated by the Affymetrix Microarray Software 5.0 (MAS 5.0) algorithm using the dChip default setting. The expression levels of only the Present probes may be considered for all quantitative analyses described below. The GEO accession number for the microarray data is GSE4309. For analysis on Illumina Human HT-12 v4 Expression Bead Chips, labeled cRNA may be hybridized according to the manufacturer's instructions.

Calculation of coverage and accuracy. A true positive is defined as probes called Present in at least six of the eight nonamplified controls, and the true expression levels are defined as the log-averaged expression levels of the Present probes. The definition of coverage is (the number of truly positive probes detected in amplified samples)/(the number of truly positive probes). The definition of accuracy is (the number of truly positive probes detected in amplified samples)/(the number of probes detected in amplified samples). The expression levels of the amplified and nonamplified samples may be divided by the class interval of 20.5 (20, 20.5, 21, 21.5 . . . ), where accuracy and coverage are calculated. These expression level bins may be also used to analyze the frequency distribution of the detected probes.

Analysis of gene expression profiles of cells: The unsupervised clustering and class neighbor analyses of the microarray data from cells may be performed using GenePattern software (http://www.broad.mit.edu/cancer/software/genepattern/), which performs the signal-to-noise ratio analysis/T-test in conjunction with the permutation test to preclude the contribution of any sample variability, including those from methodology and/or biopsy, at high confidence. The analyses may be conducted on the 14,128 probes for which at least 6 out of 20 single cells provided Present calls and at least 1 out of 20 samples provided expression levels >20 copies per cell. The expression levels calculated for probes with Absent/Marginal calls may be truncated to zero. To calculate relative gene expression levels, the Ct values obtained with Q-PCR analyses may be corrected using the efficiencies of the individual primer pairs quantified either with whole human genome (BD Biosciences) or plasmids that contain gene fragments. The relative expression levels may be further transformed into copy numbers with a calibration line calculated using the spike RNAs included in the reaction mixture (log₁₀[expression level]=1.05×log₁₀[copy number]+4.65). The Chi-square test for independence may be performed to evaluate the association of gene expressions with Gata4, which represents the difference between cluster 1 and cluster 2 determined by the unsupervised clustering and which is restricted to PE at later stages. The expression levels of individual genes measured with Q-PCR may be classified into three categories: high (>100 copies per cell), middle (10-100 copies per cell), and low (<10 copies per cell). The Chi-square and P-values for independence from Gata4 expression may be calculated based on this classification. Chi squared is defined as follows: χ2=ΣΣ(n fij−fi fj)²/n fi fj, where i and j represent expression level categories (high, middle or low) of the reference (Gata4) and the target gene, respectively; fi, fj, and fij represent the observed frequency of categories i, j and ij, respectively; and n represents the sample number (n=24). The degrees of freedom may be defined as (r−1)×(c−1), where r and c represent available numbers of expression level categories of Gata4 and of the target gene, respectively.

### Stimulating an Immune Response Against Cancer Cells

In some embodiments, antigen presenting cells (APCs) may used to activate T lymphocytes in vivo or ex vivo, to elicit an immune response against cells expressing a cancer associated sequence. APCs are highly specialized cells and may include, without limitation, macrophages, monocytes, and dendritic cells (DCs). APCs may process antigens and display their peptide fragments on the cell surface together with molecules required for lymphocyte activation. In some embodiments, the APCs may be dendritic cells. DCs may be classified into subgroups, including, e.g., follicular dendritic cells, Langerhans dendritic cells, and epidermal dendritic cells.

Some embodiments are directed to the use of cancer associated polypeptides and polynucleotides encoding a cancer associated sequence, a fragment thereof, or a mutant thereof, and antigen presenting cells (such as, without limitation, dendritic cells), to elicit an immune response against cells expressing a cancer-associated polypeptide sequence, such as, without limitation, cancer cells, in a subject. In some embodiments, the method of eliciting an immune response against cells expressing a cancer associated sequence comprises (1) isolating a hematopoietic stem cell, (2) genetically modifying the cell to express a cancer associated sequence, (3) differentiating the cell into DCs; and (4) administering the DCs to the subject (e.g., human patient). In some embodiments, the method of eliciting an immune response includes (1) isolating DCs (or isolation and differentiation of DC precursor cells), (2) pulsing the cells with a cancer associated sequence, and; (3) administering the DCs to the subject. These approaches are discussed in greater detail, infra. In some embodiments, the pulsed or expressing DCs may be used to activate T lymphocytes ex vivo. These general techniques and variations thereof may be within the skill of those in the art (see, e.g., WO97/29182; WO 97/04802; WO 97/22349; WO 96/23060; WO 98/01538; Hsu et al., 1996, Nature Med. 2:52-58), and that still other variations may be discovered in the future. In some embodiments, the cancer associated sequence is contacted with a subject to stimulate an immune response. In some embodiments, the immune response is a therapeutic immune response. In some embodiments, the immune response is a prophylactic immune response. For example, the cancer associated sequence can be contacted with a subject under conditions effective to stimulate an immune response. The cancer associated sequence can be administered as, for example, a DNA molecule (e.g. DNA vaccine), RNA molecule, or polypeptide, or any combination thereof. Administering sequence to stimulate an immune response is known, but the identity of which sequences to use was not known prior to the present disclosure. Any sequence or combination of sequences disclosed herein or a homolog thereof can be administered to a subject to stimulate an immune response.

In some embodiments, dendritic cell precursor cells are isolated for transduction with a cancer associated sequence, and induced to differentiate into dendritic cells. The genetically modified DCs express the cancer associated sequence, and may display peptide fragments on the cell surface.

In some embodiments, the cancer associated sequence expressed comprises a sequence of a naturally occurring protein. In some embodiments, the cancer associate sequence does not comprise a naturally occurring sequence. As already noted, fragments of naturally occurring proteins may be used; in addition, the expressed polypeptide may comprise mutations such as deletions, insertions, or amino acid substitutions when compared to a naturally occurring polypeptide, so long as at least one peptide epitope can be processed by the DC and presented on a MHC class I or II surface molecule. In some embodiments, it may be desirable to use sequences other than “wild type,” in order to, for example, increase antigenicity of the peptide or to increase peptide expression levels. In some embodiments, the introduced cancer associated sequences may encode variants such as polymorphic variants (e.g., a variant expressed by a particular human patient) or variants characteristic of a particular cancer (e.g., a cancer in a particular subject).

In some embodiments, a cancer associated expression sequence may be introduced (transduced) into DCs or stem cells in any of a variety of standard methods, including transfection, recombinant vaccinia viruses, adeno-associated viruses (AAVs), retroviruses, etc.

In some embodiments, the transformed DCs of the invention may be introduced into the subject (e.g., without limitation, a human patient) where the DCs may induce an immune response. Typically, the immune response includes a cytotoxic T-lymphocyte (CTL) response against target cells bearing antigenic peptides (e.g., in a MHC class I/peptide complex). These target cells are typically cancer cells.

In some embodiments, when the DCs are to be administered to a subject, they may preferably isolated from, or derived from precursor cells from, that subject (i.e., the DCs may administered to an autologous subject). However, the cells may be infused into HLA-matched allogeneic or HLA-mismatched allogeneic subject. In the latter case, immunosuppressive drugs may be administered to the subject.

In some embodiments, the cells may be administered in any suitable manner. In some embodiments, the cell may be administered with a pharmaceutically acceptable carrier (e.g., saline). In some embodiments, the cells may be administered through intravenous, intra-articular, intramuscular, intradermal, intraperitoneal, or subcutaneous routes. Administration (i.e., immunization) may be repeated at time intervals. Infusions of DC may be combined with administration of cytokines that act to maintain DC number and activity (e.g., GM-CSF, FL-12).

In some embodiments, the dose administered to a subject may be a dose sufficient to induce an immune response as detected by assays which measure T cell proliferation, T lymphocyte cytotoxicity, and/or effect a beneficial therapeutic response in the patient over time, e.g., to inhibit growth of cancer cells or result in reduction in the number of cancer cells or the size of a tumor.

In some embodiments, DCs are obtained (either from a patient or by in vitro differentiation of precursor cells) and pulsed with antigenic peptides having a cancer associated sequence. The pulsing results in the presentation of peptides onto the surface MHC molecules of the cells. The peptide/MHC complexes displayed on the cell surface may be capable of inducing a MHC-restricted cytotoxic T-lymphocyte response against target cells expressing cancer associated polypeptides (e.g., without limitations, cancer cells).

In some embodiments, cancer associated sequences used for pulsing may have at least about 6 or 8 amino acids and fewer than about 30 amino acids or fewer than about 50 amino acid residues in length. In some embodiments, an immunogenic peptide sequence may have from about 8 to about 12 amino acids. In some embodiments, a mixture of human protein fragments may be used; alternatively a particular peptide of defined sequence may be used. The peptide antigens may be produced by de novo peptide synthesis, enzymatic digestion of purified or recombinant human peptides, by purification of the peptide sequence from a natural source (e.g., a subject or tumor cells from a subject), or expression of a recombinant polynucleotide encoding a human peptide fragment.

In some embodiments, the amount of peptide used for pulsing DC may depend on the nature, size and purity of the peptide or polypeptide. In some embodiments, an amount of from about 0.05 ug/ml to about 1 mg/ml, from about 0.05 ug/ml to about 500 ug/ml, from about 0.05 ug/ml to about 250 ug/ml, from about 0.5 ug/ml to about 1 mg/ml, from about 0.5 ug/ml to about 500 ug/ml, from about 0.5 ug/ml to about 250 ug/ml, or from about 1 ug/ml to about 100 ug/ml of peptide may be used. After adding the peptide antigen(s) to the cultured DC, the cells may then be allowed sufficient time to take up and process the antigen and express antigen peptides on the cell surface in association with either class I or class II MHC. In some embodiments, the time to take up and process the antigen may be about 18 to about 30 hours, about 20 to about 30 hours, or about 24 hours.

Numerous examples of systems and methods for predicting peptide binding motifs for different MHC Class I and II molecules have been described. Such prediction could be used for predicting peptide motifs that will bind to the desired MHC Class I or II molecules. Examples of such methods, systems, and databases that those of ordinary skill in the art might consult for such purpose include:
1. Peptide Binding Motifs for MHC Class I and II Molecules; William E. Biddison, Roland Martin, Current Protocols in Immunology, Unit 11 (DOI: 10.1002/0471142735.ima01is36; Online Posting Date: May, 2001).

Reference 1 above, provides an overview of the use of peptide-binding motifs to predict interaction with a specific MHC class I or II allele, and gives examples for the use of MHC binding motifs to predict T-cell recognition.

Table 3 provides an exemplary result for a HLA peptide motif search at the NIH Center for Information Technology website, BioInformatics and Molecular Analysis Section. Full length GNGT1 peptide sequence was used as the search query.

TABLE 3
EXEMPLARY RESULT FOR HLA PEPTIDE MOTIF SEARCH
User Parameter and Scoring
Information

| | |
|---|---|
| Method selected to mimic the number of | Explicit number |
| results | |
| Number of results requested | 20 |
| HLA molecule type selected | A_0201 |
| Length selected for subsequences to be | 9 |
| scored | |
| Echoing mode selected for input sequence | Y |
| Echoing format | Numbered lines |
| Length of user's input peptide sequence | 369 |
| Number of subsequence scores calculated | 361 |
| Number of top-scoring subsequences | 20 |
| reported back in scoring output table | |

Scoring Results

| | | | Score (estimate of |
|---|---|---|---|
| | | | half time of |
| | | | disassociation of a |
| | | Subsequence residue | molecule containing |
| Rank | Start Position | listing | this subsequence |
| 1 | 310 | SLLKFLAKV (SEQ ID NO: 1) | 2249.173 |
| 2 | 183 | MLLVFGIDV (SEQ ID NO: 2) | 1662.432 |
| 3 | 137 | KVTDLVQFL (SEQ ID NO: 3) | 339.313 |
| 4 | 254 | GLYDGMMEHL (SEQ ID NO: 4) | 315.870 |
| 5 | 228 | ILILSIIFI (SEQ ID NO: 5) | 224.357 |
| 6 | 296 | FLWGPRAHA (SEQ ID NO: 6) | 189.678 |
| 7 | 245 | VIWEALNMM (SEQ ID NO: 7) | 90.891 |
| 8 | 308 | KMSILKFLA (SEQ ID NO: 8) | 72.836 |
| 9 | 166 | KNYEDHFPL (SEQ ID NO: 9) | 37.140 |
| 10 | 201 | FVLVTSLGL (SEQ ID NO: 10) | 31.814 |
| 11 | 174 | ILFSEASEC (SEQ ID NO: 11) | 31.249 |
| 12 | 213 | GMLSDVQSM (SEQ ID NO: 12) | 30.534 |
| 13 | 226 | ILILILSII (SEQ ID NO: 13) | 16.725 |
| 14 | 225 | GILILILSI (SEQ ID NO: 14) | 12.208 |
| 15 | 251 | NMMGLYDGM (SEQ ID NO: 15) | 9.758 |
| 16 | 88 | QIACSSPSV (SEQ ID NO:) | 9.563 |
| 17 | 66 | LIPSTPEEV (SEQ ID NO: 16) | 7.966 |
| 18 | 220 | SMPKTGILI (SEQ ID NO: 17) | 7.535 |
| 19 | 233 | IIFIEGYCT (SEQ ID NO: 28) | 6.445 |
| 20 | 247 | WEALNMGL (SEQ ID NO: 18) | 4.395 |

One skilled in the art of peptide-based vaccination may determine which peptides would work best in individuals based on their HLA alleles (e.g., due to “MHC restriction”). Different HLA alleles will bind particular peptide motifs (usually 2 or 3 highly conserved positions out of 8-10) with different energies which can be predicted theoretically or measured as dissociation rates. Thus, a skilled artisan may be able to tailor the peptides to a subject's HLA profile.

Introducing Cancer Associated Sequences into Cells

Some embodiments provide for antigens (e.g., cancer-associated polypeptides) associated with a variety of cancers as targets for diagnostic and/or therapeutic antibodies. These antigens may also be useful for drug discovery (e.g., small molecules) and for further characterization of cellular regulation, growth, and differentiation.

In some embodiments cells may be transfected with one or more of the cancer associated sequences disclosed herein. In some embodiments the cell may be a dendritic cell. Dendritic cells transfected with one or more of the cancer associated sequences may be used as antigen presenting cells to stimulate an immune response against one or more of the cancer associated sequences disclosed infra.

Any method known in the art may be used to transfect a cell with one or more of the cancer associated sequences disclosed infra. Electroporation may be used to introduce the cancer associated nucleic acids described herein into mammalian cells (Neumann, E. et al. (1982) EMBO J. 1, 841-845), plant and bacterial cells, and may also be used to introduce proteins (Marrero, M. B. et al. (1995) J. Biol. Chem. 270, 15734-15738; Nolkrantz, K. et al. (2002) Anal. Chem. 74, 4300-4305; Rui, M. et al. (2002) Life Sci. 71, 1771-1778). Cells (such as the cells of this invention) suspended in a buffered solution of the purified protein of interest are placed in a pulsed electrical field. Briefly, high-voltage electric pulses result in the formation of small (nanometer-sized) pores in the cell membrane. Proteins enter the cell via these small pores or during the process of membrane reorganization as the pores close and the cell returns to its normal state. The efficiency of delivery may be dependent upon the strength of the applied electrical field, the length of the pulses, temperature and the composition of the buffered medium. Electroporation is successful with a variety of cell types, even some cell lines that are resistant to other delivery methods, although the overall efficiency is often quite low. Some cell lines may remain refractory even to electroporation unless partially activated.

Microinjection may be used to introduce femtoliter volumes of DNA directly into the nucleus of a cell (Capecchi, M. R. (1980) Cell 22, 470-488) where it can be integrated directly into the host cell genome, thus creating an established cell line bearing the sequence of interest. Proteins such as antibodies (Abarzua, P. et al. (1995) Cancer Res. 55, 3490-3494; Theiss, C. and Meller, K. (2002) Exp. Cell Res. 281, 197-204) and mutant proteins (Naryanan, A. et al. (2003) J. Cell Sci. 116, 177-186) can also be directly delivered into cells via microinjection to determine their effects on cellular processes firsthand. Microinjection has the advantage of introducing macromolecules directly into the cell, thereby bypassing exposure to potentially undesirable cellular compartments such as low-pH endosomes.

Several proteins and small peptides have the ability to transduce or travel through biological membranes independent of classical receptor-mediated or endocytosis-mediated pathways. Examples of these proteins include the HIV-1 TAT protein, the herpes simplex virus 1 (HSV-1) DNA-binding protein VP22, and the Drosophila Antennapedia (Antp) homeotic transcription factor. In some embodiments, protein transduction domains (PTDs) from these proteins may be fused to other macromolecules, peptides or proteins such as, without limitation, a cancer associated polypeptide to successfully transport the polypeptide into a cell (Schwarze, S. R. et al. (2000) Trends Cell Biol. 10, 290-295). Exemplary advantages of using fusions of these transduction domains is that protein entry is rapid, concentration-dependent and appears to work with difficult cell types (Fenton, M. et al. (1998) J. Immunol. Methods 212, 41-48).

In some embodiments, liposomes may be used as vehicles to deliver oligonucleotides, DNA (gene) constructs and small drug molecules into cells (Zabner, J. et al. (1995) J. Biol. Chem. 270, 18997-19007; Feigner, P. L. et al. (1987) Proc. Natl. Acad. Sci. USA 84, 7413-7417). Certain lipids, when placed in an aqueous solution and sonicated, form closed vesicles consisting of a circularized lipid bilayer surrounding an aqueous compartment. The vesicles or liposomes of embodiments herein may be formed in a solution containing the molecule to be delivered. In addition to encapsulating DNA in an aqueous solution, cationic liposomes may spontaneously and efficiently form complexes with DNA, with the positively charged head groups on the lipids interacting with the negatively charged backbone of the DNA. The exact composition and/or mixture of cationic lipids used can be altered, depending upon the macromolecule of interest and the cell type used (Feigner, J. H. et al. (1994) J. Biol. Chem. 269, 2550-2561). The cationic liposome strategy has also been applied successfully to protein delivery (Zelphati, O. et al. (2001) J. Biol. Chem. 276, 35103-35110). Because proteins are more heterogeneous than DNA, the physical characteristics of the protein, such as its charge and hydrophobicity, may influence the extent of its interaction with the cationic lipids.

### Capture Reagents and Binding Partners

In certain embodiments of the invention provides capture reagents and specific binding partners for molecules encoded by the cancer associated sequences disclosed infra. The capture reagents and specific binding partners may be used to isolate a molecule, such as a nucleic acid encoding a cancer associated sequence disclosed infra, or a protein or protein fragment encoded by cancer associated sequence disclosed infra. The capture reagent and specific binding partners may be used to diagnose and/or detect cancer in a sample. The capture reagent or binding partner may be used as therapeutic to treat cancer, where the cancer is associated with the expression of one or more cancer associated sequences disclosed infra.

The capture reagent and specific binding partner may be any molecule that specifically binds to a molecule encoded for by cancer associated sequence disclosed infra. The may be a protein, peptide, a nucleic acid, including DNA, RNA, PNA and the like, a lipid, a carbohydrate, a small molecule, including inorganic and organic molecules and combination of a plurality of the foregoing molecules.

The capture reagent and binding partner may be, for example, a nucleic acid such as DNA molecule or a PNA molecule. The nucleic acid may bind to a sequence encoded for by cancer associated sequence, such as a DNA molecule, or an RNA molecule. The capture reagent or binding partner may be, for example 5-500 nucleotides long, 10-200 nucleotides long, 20-100 nucleotides long. The capture reagent or binding partner may be about 5, about 10, about 15, about 20, about 25, about 30, about 35, about 40 nucleotides long.

The capture reagent and specific binding partner may be, for example, a protein, including any protein that binds specifically to a molecule encoded for by cancer associated sequence. As an example, the capture reagent and specific binding partner may be an antibody. The antibody, may, for example bind to a protein or protein fragment encoded for by a cancer associated sequence.

Binding in IgG antibodies, for example, is generally characterized by an affinity of at least about 10⁻⁷ M or higher, such as at least about 10⁻⁸ M or higher, or at least about 10⁻⁹ M or higher, or at least about 10⁻¹⁰ or higher, or at least about 10⁻¹¹ M or higher, or at least about 10⁻¹² M or higher. The term is also applicable where, e.g., an antigen-binding domain is specific for a particular epitope that is not carried by numerous antigens, in which case the antibody or antigen binding protein carrying the antigen-binding domain will generally not bind other antigens. In some embodiments, the capture reagent has a Kd equal or less than 10⁻⁹ M, 10⁻¹⁰ M, or 10⁻¹¹ M for its binding partner (e.g. antigen). In some embodiments, the capture reagent has a Ka greater than or equal to 10⁹ M⁻¹ for its binding partner. Capture reagent can also refer to, for example, antibodies. Intact antibodies, also known as immunoglobulins, are typically tetrameric glycosylated proteins composed of two light (L) chains of approximately 25 kDa each, and two heavy (H) chains of approximately 50 kDa each. Two types of light chain, termed lambda and kappa, exist in antibodies. Depending on the amino acid sequence of the constant domain of heavy chains, immunoglobulins are assigned to five major classes: A, D, E, G, and M, and several of these may be further divided into subclasses (isotypes), e.g., IgG1, IgG2, IgG3, IgG4, IgA1, and IgA2. Each light chain is composed of an N-terminal variable (V) domain (VL) and a constant (C) domain (CL). Each heavy chain is composed of an N-terminal V domain (VH), three or four C domains (CHs), and a hinge region. The CH domain most proximal to VH is designated CH1. The VH and VL domains consist of four regions of relatively conserved sequences named framework regions (FR1, FR2, FR3, and FR4), which form a scaffold for three regions of hypervariable sequences (complementarity determining regions, CDRs). The CDRs contain most of the residues responsible for specific interactions of the antibody or antigen binding protein with the antigen. CDRs are referred to as CDR1, CDR2, and CDR3. Accordingly, CDR constituents on the heavy chain are referred to as H1, H2, and H3, while CDR constituents on the light chain are referred to as L1, L2, and L3. CDR3 is the greatest source of molecular diversity within the antibody or antigen binding protein-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, Eds. Harlow et al., 1988. One of skill in the art will recognize that each subunit structure, e.g., a CH, VH, CL, VL, CDR, and/or FR structure, comprises active fragments. For example, active fragments may consist of the portion of the VH, VL, or CDR subunit that binds the antigen, i.e., the antigen-binding fragment, or the portion of the CH subunit that binds to and/or activates an Fc receptor and/or complement.

Non-limiting examples of binding fragments encompassed within the term “antigen-specific antibody” used herein include: (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment, which consists of a VH domain; and (vi) an isolated CDR. Furthermore, although the two domains of the Fv fragment, VL and VH, are coded for by separate genes, they may be recombinantly joined by a synthetic linker, creating a single protein chain in which the VL and VH domains pair to form monovalent molecules (known as single chain Fv (scFv)). The most commonly used linker is a 15-residue (Gly₄Ser)₃ peptide, but other linkers are also known in the art. Single chain antibodies are also intended to be encompassed within the terms “antibody or antigen binding protein,” or “antigen-binding fragment” of an antibody. The antibody can also be a polyclonal antibody, monoclonal antibody, chimeric antibody, antigen-binding fragment, Fc fragment, single chain antibodies, or any derivatives thereof.

Antibodies can be obtained using conventional techniques known to those skilled in the art, and the fragments are screened for utility in the same manner as intact antibodies. Antibody diversity is created by multiple germline genes encoding variable domains and a variety of somatic events. The somatic events include recombination of variable gene segments with diversity (D) and joining (J) gene segments to make a complete VH domain, and the recombination of variable and joining gene segments to make a complete VL domain. The recombination process itself is imprecise, resulting in the loss or addition of amino acids at the V(D)J junctions. These mechanisms of diversity occur in the developing B cell prior to antigen exposure. After antigenic stimulation, the expressed antibody genes in B cells undergo somatic mutation. Based on the estimated number of germline gene segments, the random recombination of these segments, and random VH-VL pairing, up to 1.6×10⁷ different antibodies may be produced (Fundamental Immunology, 3rd ed. (1993), ed. Paul, Raven Press, New York, N.Y.). When other processes that contribute to antibody diversity (such as somatic mutation) are taken into account, it is thought that upwards of 1×10¹⁰ different antibodies may be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

Antibody or antigen binding protein molecules capable of specifically interacting with the antigens, epitopes, or other molecules described herein may be produced by methods well known to those skilled in the art. For example, monoclonal antibodies can be produced by generation of hybridomas in accordance with known methods. Hybridomas formed in this manner can then be screened using standard methods, such as enzyme-linked immunosorbent assay (ELISA) and Biacore analysis, to identify one or more hybridomas that produce an antibody that specifically interacts with a molecule or compound of interest. As an alternative to preparing monoclonal antibody-secreting hybridomas, a monoclonal antibody to a polypeptide of the present disclosure may be identified and isolated by screening a recombinant combinatorial immunoglobulin library (e.g., an antibody phage display library) with a polypeptide of the present disclosure to thereby isolate immunoglobulin library members that bind to the polypeptide. Techniques and commercially available kits for generating and screening phage display libraries are well known to those skilled in the art. Additionally, examples of methods and reagents particularly amenable for use in generating and screening antibody or antigen binding protein display libraries can be found in the literature.

Examples of chimeric antibodies include, but are not limited to, humanized antibodies. The antibodies described herein can also be human antibodies. In some embodiments, the capture reagent comprises a detection reagent. The detection reagent can be any reagent that can be used to detect the presence of the capture reagent binding to its specific binding partner. The capture reagent can comprise a detection reagent directly or the capture reagent can comprise a particle that comprises the detection reagent. In some embodiments, the capture reagent and/or particle comprises a color, colloidal gold, radioactive tag, fluorescent tag, or a chemiluminescent substrate. The particle can be, for example, a viral particle, a latex particle, a lipid particle, or a fluorescent particle.

The capture reagents (e.g. antibody) of the present disclosure can also include an anti-antibody, i.e. an antibody that recognizes another antibody but is not specific to an antigen, such as, but not limited to, anti-IgG, anti-IgM, or ant-IgE antibody. This non-specific antibody can be used as a positive control to detect whether the antigen specific antibody is present in a sample. In some embodiments, the antibody binds to an epitope from a protein encoded by the nucleotide sequence disclosed in Table 1. In some embodiments, the epitope is a fragment of the protein sequence encoded by the nucleotide sequence of a sequence disclosed in Table 1, which is described herein. In some embodiments, the epitope comprises about 1-10, 1-20, 1-30, 3-10, or 3-15 residues of the cancer associated sequence. In some embodiments, the epitope is not linear.

In some embodiments, the antibody binds to the regions described herein or a peptide with at least 90, 95, or 99% homology or identity to the region. In some embodiments, the fragment of the regions described herein is 5-10 residues in length. In some embodiments, the fragment of the regions (e.g. epitope) described herein are 3-5 residues in length. The fragments are described based upon the length provided. In some embodiments, the epitope is about 3, 4, 5, 6, 7, 8, 9, 10, 11, 12, 13, 14, 15, or 20 residues in length.

In some embodiments, the sequence to which the antibody binds may include both nucleic acid and amino acid sequences. In some embodiments, the sequence to which the antibody binds may include sequences having at least about 60% homology with the disclosed sequences. In some embodiments, the sequence to which the antibody binds may have at least about 65%, about 70%, about 75%, about 80%, about 85%, about 90%, about 95%, about 97%, about 99%, about 99.8% homology with the disclosed sequences. In some embodiments, the sequences may be referred to as “mutant nucleic acids” or “mutant peptide sequences.”

### Treatment of Cancer

In some embodiments, cancers expressing one or more of the cancer associated sequences may be treated by antagonizing the cancer associated sequence's activity. In some embodiments, a method of treating cancer may comprise administering a therapeutic such as, without limitation, antibodies that antagonize the ligand binding to the cancer associated sequence, small molecules that inhibit the cancer associated sequence's expression or activity, siRNAs directed towards the cancer associated sequence, or the like. In some embodiments, a method of treating cancer may comprise administering an antibody against the protein to a subject in need thereof. In some embodiments, the antibody may be a monoclonal antibody or a polyclonal antibody. In some embodiments, the antibody may be a humanized or a recombinant antibody. Antibodies can be made that specifically bind to this region using known methods and any method is suitable. In some embodiments, the antibody specifically binds to a sequence disclosed in Table 1 or a fragment thereof.

In some embodiments, a method of treating cancer (e.g. adenocarcinomas or other types of cancer) comprises detecting the presence of a cancer associated sequence's receptor and administering a cancer treatment. The cancer treatment may be any cancer treatment or one that is specific to the inhibiting the action of a cancer associated sequence. For example, various cancers are tested to determine if a specific molecule is present before giving a cancer treatment. In some embodiments, therefore, a sample would be obtained from the patient and tested for the presence of a cancer associated sequence or the overexpression of a cancer associated sequence as described herein. In some embodiments, if a cancer associated sequence is found to be overexpressed, a cancer treatment or therapeutic is administered to the subject. The cancer treatment may be a conventional non-specific treatment, such as chemotherapy, or the treatment may comprise of a specific treatment that only targets the activity of the cancer associated sequence or the receptor to which the cancer associated sequence binds. These treatments can be, for example, an antibody that specifically binds to the cancer associated sequence and inhibits its activity.

In some embodiments, the present disclosure provides methods of treating cancer in a subject, the method comprising administering to a subject having cancer an agent that inhibits activity of any cancer associated sequence disclosed infra. In some embodiments the invention provides methods of treating cancer in a subject, the method comprising administering to a subject having cancer an agent that inhibits activity of one or more of gene expression and or protein expression encoded for by GNGT1, C12orf56, COL10A1, SLC35D3, snaR-A, SBK1, DSCR8, CELSR3 or a complement thereof.

In some embodiments, the cancer cell may be targeted specifically with a therapeutic based upon the differentially expressed gene or gene product. For example, in some embodiments, the differentially expressed gene product may be an enzyme, which can convert an anti-cancer prodrug into its active form. Therefore, in normal cells, where the differentially expressed gene product is not expressed or expressed at significantly lower levels, the prodrug may be either not activated or activated in a lesser amount, and may be, therefore less toxic to normal cells. Therefore, the cancer prodrug may, in some embodiments, be given in a higher dosage so that the cancer cells can metabolize the prodrug, which will, for example, kill the cancer cell, and the normal cells will not metabolize the prodrug or not as well, and, therefore, be less toxic to the patient. An example of this is where tumor cells overexpress a metalloprotease, which is described in Atkinson et al., British Journal of Pharmacology (2008) 153, 1344-1352, which is hereby incorporated by reference in its entirety and for the method of specifically targeting cancer cells. Using proteases to target cancer cells is also described in Carl et al., PNAS, Vol. 77, No. 4, pp. 2224-2228, April 1980, which is hereby incorporated by reference in its entirety and for the method of specifically targeting cancer cells. For example, doxorubicin or other type of chemotherapeutic can be linked to a peptide sequence that is specifically cleaved or recognized by the differentially expressed gene product. The doxorubicin or other type of chemotherapeutic is then cleaved from the peptide sequence and is activated such that it can kill or inhibit the growth of the cancer cell whereas in the normal cell the chemotherapeutic is never internalized into the cell or is not metabolized as efficiently, and is, therefore, less toxic.

In some embodiments, a method of treating cancer may comprise gene knockdown of one or more cancer associated sequences described herein. Gene knockdown refers to techniques by which the expression of one or more of an organism's genes is reduced, either through genetic modification (a change in the DNA of one of the organism's chromosomes such as, without limitation, chromosomes encoding cancer associated sequences) or by treatment with a reagent such as a short DNA or RNA oligonucleotide with a sequence complementary to either an mRNA transcript or a gene. In some embodiments, the oligonucleotide used may be selected from RNase-H competent antisense, such as, without limitation, ssDNA oligonucleotides, ssRNA oligonucleotides, phosphorothioate oligonucleotides, or chimeric oligonucleotides; RNase-independent antisense, such as morpholino oligonucleotides, 2′-O-methyl phosphorothioate oligonucleotides, locked nucleic acid oligonucleotides, or peptide nucleic acid oligonucleotides; RNAi oligonucleotides, such as, without limitation, siRNA duplex oligonucleotides, or shRNA oligonucleotides; or any combination thereof. In some embodiments, a plasmid may be introduced into a cell, wherein the plasmid expresses either an antisense RNA transcript or an shRNA transcript. The oligo introduced or transcript expressed may interact with the target mRNA (ex. a sequence disclosed in Table 1) by complementary base pairing (a sense-antisense interaction).

The specific mechanism of silencing may vary with the oligo chemistry. In some embodiments, the binding of a oligonucleotide described herein to the active gene or its transcripts may cause decreased expression through blocking of transcription, degradation of the mRNA transcript (e.g. by small interfering RNA (siRNA) or RNase-H dependent antisense) or blocking either mRNA translation, pre-mRNA splicing sites or nuclease cleavage sites used for maturation of other functional RNAs such as miRNA (e.g. by Morpholino oligonucleotides or other RNase-H independent antisense). For example, RNase-H competent antisense oligonucleotides (and antisense RNA transcripts) may form duplexes with RNA that are recognized by the enzyme RNase-H, which cleaves the RNA strand. As another example, RNase-independent oligonucleotides may bind to the mRNA and block the translation process. In some embodiments, the oligonucleotides may bind in the 5′-UTR and halt the initiation complex as it travels from the 5′-cap to the start codon, preventing ribosome assembly. A single strand of RNAi oligonucleotides may be loaded into the RISC complex, which catalytically cleaves complementary sequences and inhibits translation of some mRNAs bearing partially-complementary sequences. The oligonucleotides may be introduced into a cell by any technique including, without limitation, electroporation, microinjection, salt-shock methods such as, for example, CaCl2 shock; transfection of anionic oligo by cationic lipids such as, for example, Lipofectamine; transfection of uncharged oligonucleotides by endosomal release agents such as, for example, Endo-Porter; or any combination thereof. In some embodiments, the oligonucleotides may be delivered from the blood to the cytosol using techniques selected from nanoparticle complexes, virally-mediated transfection, oligonucleotides linked to octaguanidinium dendrimers (Morpholino oligonucleotides), or any combination thereof.

In some embodiments, a method of treating cancer may comprise treating cells to knockdown or inhibit expression of a gene encoding the mRNA disclosed in Table 1. The method may comprise culturing hES cell-derived clonal embryonic progenitor cell lines CM02 and EN13 (see U.S. Patent Publication 2008/0070303, entitled “Methods to accelerate the isolation of novel cell strains from pluripotent stem cells and cells obtained thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each of which is incorporated by reference herein in its entirety) with a retrovirus expressing silencing RNA directed to a cancer-associated sequence. In some embodiments, the method may further comprise confirming down-regulation by qPCR. In some embodiments, the method further comprises cryopreserving the cells. In some embodiments, the method further comprises reprogramming the cells. In some embodiments, the method comprises cryopreserving or reprogramming the cells within two days by the exogenous administration of OCT4, MYC, KLF4, and SOX2 (see Takahashi and Yamanaka 2006 Aug. 25; 126(4):663-76; U.S. patent application Ser. No. 12/086,479, published as US2009/0068742 and entitled “Nuclear Reprogramming Factor”, each of which is incorporated herein by reference) and by the method described in PCT/US06/30632, published as WO/2007/019398 and entitled “Improved Methods of Reprogramming Animal Somatic Cells”, incorporated by reference herein in its entirety. In some embodiments, the method may comprise culturing mammalian differentiated cells under conditions that promote the propagation of ES cells. In some embodiments, any convenient ES cell propagation condition may be used, e.g., on feeders or in feeder free media capable of propagating ES cells. In some embodiments, the method comprises identifying cells from ES colonies in the culture. Cells from the identified ES colony may then be evaluated for ES markers, e.g., Oct4, TRA 1-60, TRA 1-81, SSEA4, etc., and those having ES cell phenotype may be expanded. Control lines that have not been preconditioned by the knockdown may be reprogrammed in parallel to demonstrate the effectiveness of the preconditioning.

Some embodiments herein are directed to a method of treating cancer in a subject, the method comprising administering to a subject in need thereof a therapeutic agent modulating the activity of a cancer associated protein, wherein the cancer associated protein is encoded by a nucleic acid comprising a nucleic acid sequence selected from a sequence disclosed in Table 1, homologs thereof, combinations thereof, or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody. In some embodiments, a method of treating cancer may comprise gene knockdown of a gene disclosed in Table 1. In some embodiments, a method of treating cancer may comprise treating cells to knockdown or inhibit expression of a gene encoding the mRNA disclosed in Table 1.

In some embodiments, a method of treating cancer may comprise administering an agent that interferes with the synthesis, secretion, receptor binding or receptor signaling of cancer associated proteins (e.g. a protein encoded for by one or more of the cancer associated sequences disclosed infra.) or its receptors.

In some embodiments, the cancers treated by modulating the activity or expression of the genes disclosed in Table 1 or the gene product thereof is a cancer classified by site or by histological type.

In some embodiments, implementation of an immunotherapy strategy for treating, reducing the symptoms of, or preventing cancer or neoplasms, (e.g., a vaccine) may be achieved using many different techniques available to the skilled artisan.

Immunotherapy or the use of antibodies for therapeutic purposes has been used in recent years to treat cancer. Passive immunotherapy involves the use of monoclonal antibodies in cancer treatments. See, for example, Cancer: Principles and Practice of Oncology, 6 th Edition (2001) Chapt. 20 pp. 495-508. Inherent therapeutic biological activity of these antibodies include direct inhibition of tumor cell growth or survival, and the ability to recruit the natural cell killing activity of the body's immune system. These agents may be administered alone or in conjunction with radiation or chemotherapeutic agents. Alternatively, antibodies may be used to make antibody conjugates where the antibody is linked to a toxic agent and directs that agent to the tumor by specifically binding to the tumor.

In some embodiments, a method for treating cancer comprises administering to a subject in need thereof a therapeutic agent modulating the activity of a cancer associated protein, wherein the cancer associated protein is encoded by a nucleic acid comprising a nucleic acid sequence selected from the group consisting of the human nucleic acid sequences in Table 1 and further wherein the therapeutic agent binds to the cancer associated protein.

In some embodiments, a method of treating cancer comprises administering an antibody (e.g. monoclonal antibody, human antibody, humanized antibody, recombinant antibody, chimeric antibody, and the like) that specifically binds to a cancer associated protein that is expressed on a cell surface. In some embodiments, the antibody binds to an extracellular domain of the cancer associated protein. In some embodiments, the antibody binds to a cancer associated protein differentially expressed on a cancer cell surface relative to a normal cell surface, or, in some embodiments, to at least one human cancer cell line. In some embodiments, the antibody is linked to a therapeutic agent. Kits and pharmaceutical compositions for detecting a presence or an absence of cancer cells in a subject, and comprising such antibodies are also provided.

### Administration of Therapeutics and Pharmaceutical Compositions

Modes of administration for a therapeutic (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of therapeutic to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the therapeutic of the present disclosure and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present disclosure. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compositions of the present disclosure can be formulated for parenteral administration by injection, e.g., by bolus injection or continuous infusion. The compositions can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compositions can be formulated readily by combining the therapeutic with pharmaceutically acceptable carriers well known in the art. Such carriers enable the therapeutic of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active therapeutic doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active therapeutic can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the pharmaceutical compositions can take the form of, e.g., tablets or lozenges formulated in a conventional manner.

For administration by inhalation, the therapeutic for use according to the present disclosure is conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the therapeutic and a suitable powder base such as lactose or starch.

The compositions of the present disclosure can also be formulated in rectal compositions such as suppositories or retention enemas, e.g., containing conventional suppository bases such as cocoa butter or other glycerides.

In addition to the formulations described previously, the therapeutic of the present disclosure can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compositions can be formulated with suitable polymeric or hydrophobic materials (for example as an emulsion in an acceptable oil) or ion exchange resins, or as sparingly soluble derivatives, for example, as a sparingly soluble salt.

In transdermal administration, the compositions of the present disclosure, for example, can be applied to a plaster, or can be applied by transdermal, therapeutic systems that are consequently supplied to the organism.

Pharmaceutical compositions can include suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compositions of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component may include one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearylfumarate, fatty acid, fatty alcohol, fatty acid ester, glycerylbehenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### Kits

Also provided by the subject invention are kits and systems for practicing the subject methods, as described above, such components configured to diagnose cancer in a subject, treat cancer in a subject, or perform basic research experiments on cancer cells (e.g., either derived directly from a subject, grown in vitro or ex vivo, or from an animal model of cancer. The various components of the kits may be present in separate containers or certain compatible components may be pre-combined into a single container, as desired.

In some embodiments, the invention provides a kit for diagnosing the presence of cancer in a test sample, said kit comprising at least one polynucleotide that selectively hybridizes to a cancer associated polynucleotide sequence shown in Table 1, or its complement. In another embodiment the invention provides an electronic library comprising a cancer associated polynucleotide, a cancer associated polypeptide, or fragment thereof, shown in Table 1.

The subject systems and kits may also include one or more other reagents for performing any of the subject methods. The reagents may include one or more matrices, solvents, sample preparation reagents, buffers, desalting reagents, enzymatic reagents, denaturing reagents, probes, polynucleotides, vectors (e.g., plasmid or viral vectors), etc., where calibration standards such as positive and negative controls may be provided as well. As such, the kits may include one or more containers such as vials or bottles, with each container containing a separate component for carrying out a sample processing or preparing step and/or for carrying out one or more steps for producing a normalized sample according to the present disclosure.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the subject database, programming and instructions, the kits may also include one or more control samples and reagents, e.g., two or more control samples for use in testing the kit.

### Additional Embodiments of the Invention

Table 2 provided infra shows raw data from an Illumina microarray screen.

Embodiments of the disclosure are directed to methods of diagnosis, prognosis and treatment of cancer. The methods, compositions and kits described herein may be used for the treatment, diagnosis and prognosis of cancer including any cancer disclosed herein

In some embodiments, the methods comprise targeting a marker that is expressed at abnormal levels in tumor tissue in comparison to normal somatic tissue. In some embodiments, the marker may comprise a sequence selected from a sequence disclosed in Table 1, a complement thereof, or a combination thereof. In some embodiments, the methods for the treatment of cancer and related pharmaceutical preparations and kits are provided. Some embodiments are directed to methods of treating cancer comprising administering a composition including a therapeutic that affects the expression, abundance or activity of a target marker. In some embodiments, the target marker may include a sequence described in Table 1, a complement thereof, or any combination thereof.

Some embodiments are directed to methods of detecting cancer comprising detecting a level of a target marker associated with the cancer. In some embodiments, the target marker may include a sequence described in Table 1, a complement thereof or any combination thereof.

Some embodiments herein provide antigens (i.e. cancer-associated polypeptides) associated with cancer as targets for diagnostic and/or therapeutic antibodies. In some embodiments, these antigens may be useful for drug discovery (e.g., small molecules) and for further characterization of cellular regulation, growth, and differentiation.

Some embodiments describe a method of diagnosing cancer in a subject, the method comprising: (a) determining the expression of one or more genes or gene products or homologs thereof disclosed infra e.g. disclosed in Table 1 and/or infra under the heading Cancer Associated Sequences; and (b) comparing the expression of the one or more nucleic acid sequences from a second normal sample from the first subject or a second unaffected subject, wherein a difference in the expression indicates that the first subject has cancer. Some embodiments describe a method of eliciting an immune response against cells expressing a cancer associated sequence comprising contacting a subject with a cancer associated sequence under conditions effective to elicit an immune response in the subject, wherein the cancer associated sequence comprises a sequence or fragment thereof a gene selected from a gene described in Table 1 or a combination thereof.

Some embodiments describe a method of detecting cancer in a test sample, comprising: (i) detecting a level of activity of at least one polypeptide that is a gene product; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein the gene product is a product of a gene selected from: a gene described in Table 1 or a combination thereof.

Some embodiments herein are directed to a method of treating cancer in a subject, the method comprising administering to a subject in need thereof a therapeutic agent modulating the activity of a cancer associated protein, wherein the cancer associated protein is encoded by a nucleic acid comprising a nucleic acid sequence selected from a sequence described in Table 1, homologs thereof, combinations thereof, or a fragment thereof. In some embodiments, the therapeutic agent binds to the cancer associated protein. In some embodiments, the therapeutic agent is an antibody. In some embodiments, the antibody may be a monoclonal antibody or a polyclonal antibody. In some embodiments, the antibody is a humanized or human antibody. In some embodiments, a method of treating cancer may comprise gene knockdown of a gene described in Table 1. In some embodiments, a method of treating cancer may comprise treating cells to knockdown or inhibit expression of a gene encoding an mRNA disclosed in Table 1. The methods disclosed herein may also be used for diagnosis and treatment of other conditions in which cells have become immortalized.

In some embodiments, a method of diagnosing a subject with cancer comprises obtaining a sample and detecting the presence of a cancer associated sequence selected from a sequence described in Table 1, a fragment thereof or a complement thereof wherein the presence of the cancer associated sequence indicates the subject has cancer. In some embodiments, detecting the presence of a cancer associated sequence comprises contacting the sample with an antibody or other type of capture reagent that specifically binds to the cancer associated sequence's protein and detecting the presence or absence of the binding to the cancer associated sequence's protein in the sample. The methods disclosed herein may also be used for diagnosis and treatment of other conditions in which cells have become immortalized.

In some embodiments, the present invention provides methods of treating cancer in a subject, the method comprising administering to a subject in need thereof a therapeutic agent that modulates the activity of a sequence disclosed in Table 1 or homologs thereof, wherein the therapeutic agent treats the cancer in the subject.

In some embodiments, the present invention provides methods of diagnosing cancer in a subject, the method comprising determining the expression of a gene disclosed in Table 1 from a sample; and diagnosing cancer in the subject based on the expression, wherein the subject is diagnosed as having cancer if the gene disclosed in Table 1 is overexpressed.

In some embodiments, the present invention provides methods of detecting cancer in a test sample, the method comprising: (i) detecting a level of an antibody, wherein the antibody binds to an antigenic polypeptide encoded by a nucleic acid sequence comprising a sequence disclosed in Table 1, homologs thereof, combinations thereof, or a fragment thereof; and (ii) comparing the level of the antibody in the test sample with a level of the antibody in a control sample, wherein an altered level of antibody in the test sample relative to the level of antibody in the control sample is indicative of the presence of cancer in the test sample.

In some embodiments, the present invention provides methods of detecting cancer in a test sample, comprising: (i) detecting a level of activity of at least one polypeptide that is encoded by a nucleic acid comprising a nucleic acid sequence disclosed in Table 1, homologs thereof; combinations thereof, or a fragment thereof; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample.

In some embodiments, the present invention provides methods of detecting cancer in a test sample, the method comprising: (i) detecting a level of expression of at least one polypeptide that is encoded by a nucleic acid comprising a nucleic acid sequence disclosed in Table 1, homologs thereof, combinations thereof, or a fragment thereof; and (ii) comparing the level of expression of the polypeptide in the test sample with a level of expression of polypeptide in a normal sample, wherein an altered level of expression of the polypeptide in the test sample relative to the level of polypeptide expression in the normal sample is indicative of the presence of cancer in the test sample.

In some embodiments, the present invention provides methods of detecting cancer in a test sample, the method comprising: (i) detecting a level of expression of a nucleic acid sequence comprising a nucleic acid sequence disclosed in Table 1, homologs thereof, mutant nucleic acids thereof, combinations thereof; or a fragment thereof; and (ii) comparing the level of expression of the nucleic acid sequence in the test sample with a level of expression of nucleic acid sequence in a normal sample, wherein an altered level of expression of the nucleic acid sequence in the test sample relative to the level of nucleic acid sequence expression in the normal sample is indicative of the presence of cancer in the test sample.

In some embodiments, the present invention provides methods of screening for activity against cancer, the method comprising: (a) contacting a cell that expresses a cancer associated gene comprising a sequence disclosed in Table 1, a complement thereof, homologs thereof, combinations thereof, or fragments thereof with a cancer drug candidate; (b) detecting an effect of the cancer drug candidate on an expression of the cancer associated polynucleotide in the cell; and (c) comparing the level of expression in the absence of the drug candidate to the level of expression in the presence of the drug candidate; wherein an effect on the expression of the cancer associate polynucleotide indicates that the candidate has activity against cancer.

In some embodiments, the present invention provides methods of screening for activity against cancer, the method comprising: (a) contacting a cell that overexpresses a cancer associated gene comprising a sequence disclosed in Table 1, a complement thereof, homologs thereof, combinations thereof, or fragments thereof with a cancer drug candidate; (b) detecting an effect of the cancer drug candidate on an expression of the cancer associated polynucleotide in the cell or an effect on cell growth or viability; and (c) comparing the level of expression, cell growth, or viability in the absence of the drug candidate to the level of expression, cell growth, or viability in the presence of the drug candidate; wherein an effect on the expression of the cancer associated polynucleotide, cell growth, or viability indicates that the candidate has activity against cancer cell that overexpresses a cancer associated gene comprising the sequence disclosed in Table 1, a complement thereof, homologs thereof, combinations thereof, or fragments thereof.

In some embodiments, the present invention provides methods of diagnosing cancer in a subject, the method comprising: a) determining the expression of one or more nucleic acid sequences, wherein the one or more nucleic acid sequences comprises a sequence disclosed in Table 1, homologs thereof, combinations thereof, or fragments thereof in a first sample of a first subject; and b) comparing the expression of the one or more nucleic acid sequences from a second normal sample from the first subject or a second unaffected subject, wherein a difference in the expression of a sequence disclosed in Table 1 indicates that the first subject has cancer.

In some embodiments, the present invention provides methods of diagnosing cancer in a subject, the method comprising: a) determining the expression of one or more genes or gene products or homologs thereof in a subject; and b) comparing the expression of the one or more genes or gene products or homologs thereof in the subject to the expression of one or more genes or gene products or homologs thereof from a normal sample from the subject or a normal sample from an unaffected subject, wherein a difference in the expression indicates that the subject has cancer, wherein the one or more genes or gene products comprises a sequence disclosed in Table 1.

In some embodiments, the present invention provides methods of detecting cancer in a test sample, comprising: (i) detecting a level of activity of at least one polypeptide; and (ii) comparing the level of activity of the polypeptide in the test sample with a level of activity of polypeptide in a normal sample, wherein an altered level of activity of the polypeptide in the test sample relative to the level of polypeptide activity in the normal sample is indicative of the presence of cancer in the test sample, wherein the polypeptide is a gene product of a sequence disclosed in Table 1.

In some embodiments, the present invention provides methods of diagnosing cancer in a subject, the method comprising: obtaining one or more gene expression results for one or more sequences, wherein the one or more sequences comprises a sequence disclosed in Table 1, from a sample derived from a subject; and diagnosing cancer in the subject based on the one or more gene expression results, wherein the subject is diagnosed as having cancer if one or more genes is overexpressed.

Embodiments illustrating the method and materials used may be further understood by reference to the following non-limiting examples.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### GNGT1

GNGT1 (Accession number NM_{—}021955.3) encodes a “guanine nucleotide binding protein (G protein) gamma transducing activity polypeptide 1”, the gamma subunit of transducing, a G-protein found specifically in rod outer segments, where it mediates the activation by rhodopsin of a cyclic GTP-specific phosphodiesterase (Hurley et al., 1984 [PubMed 6438626]; Scherer et al., 1996 [PubMed 8661128]). Surprisingly, the data included herein shows that GNGT1 is a novel marker for many types of malignant tumors from diverse tissues of origin, including, but not limited to, tumors of the kidney, cervix, endometrium, ovary, lung, bladder, liver, breast, soft tissue, connective tissue, stomach, esophagus, uterus, and muscle. Therefore, as discussed herein and throughout, GNGT1 can be used as a diagnostic marker in a subject. GNGT1 can be used to diagnose cancer in a subject.

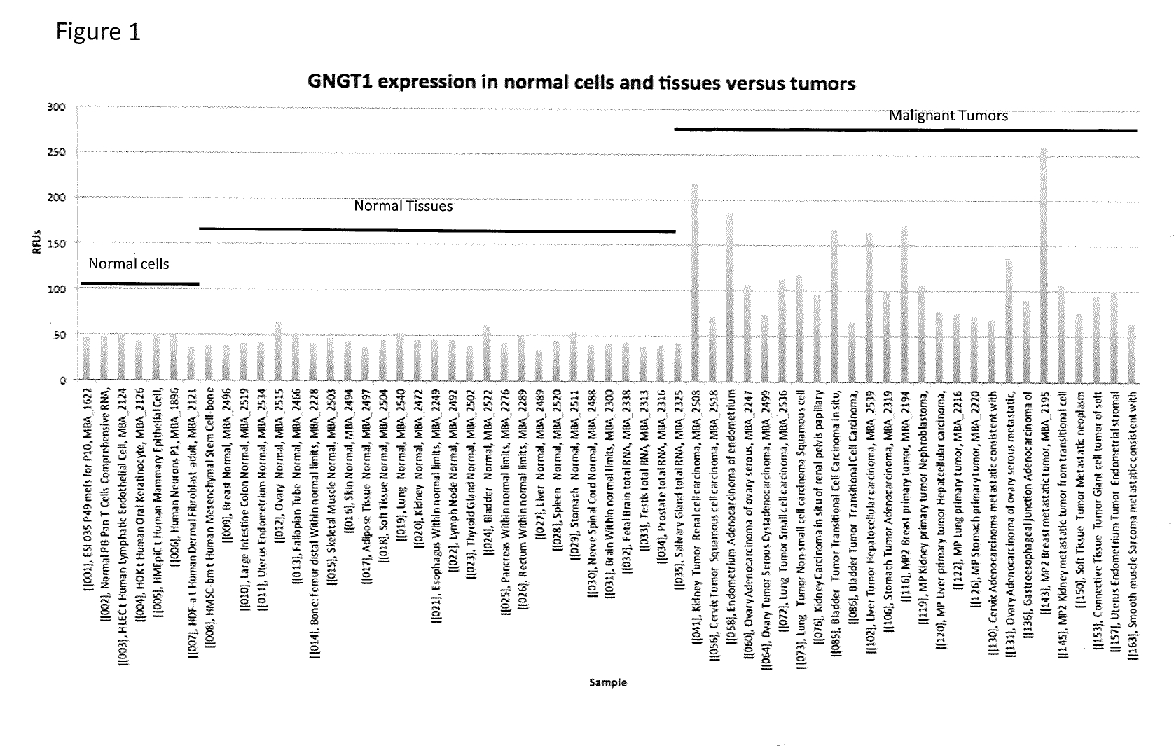

As shown in FIG. 1, GNGT1 expression was assayed by Illumina microarray, a probe specific for GNGT1 (probe sequence GTTGAAGAACGATCTGGCGAGGATCCACT GGTAAAGGGCATCCCAGAGGA; (SEQ ID NO: 19) Illumina probe ID ILMN_{—}2091100). The assay detected strong gene expression (>75 rfus) in kidney tumor, renal cell carcinoma, cervix tumor, aquamous cell carcinoma, endometrium adenocarcinoma of endometrium endometrioid, Ovary Adenocarcinoma of ovary serous, Ovary Tumor Serous Cystadenocarcinoma, Lung Tumor Small cell carcinoma, Lung Tumor Non-small cell carcinoma, Squamous cell carcinoma, Kidney Carcinoma in situ of renal pelvis papillary transitional cell, Bladder Tumor Transitional Cell Carcinoma in situ, Bladder Tumor Transitional Cell Carcinoma, Liver Tumor Hepatocellular carcinoma, Stomach Tumor Adenocarcinoma, Breast primary tumor, Kidney primary tumor Nephroblastoma, Liver primary tumor Hepatcellular carcinoma, Lung primary tumor, Stomach primary tumor, Cervix Adenocarcinoma metastatic, Ovary Adenocarcinoma of ovary serous metastatic, Adenocarcinoma of gastroesophageal junction metastatic, Breast metastatic tumor, Kidney metastatic tumor from transitional cell carcinoma, Soft Tissue Tumor Metastatic neoplasm adenocarcinoma Serous cystadenocarcinoma, Connective Tissue Tumor Giant cell tumor of soft parts malignant, Uterus Endometrium Tumor Endometrial stromal sarcoma, and Smooth muscle Sarcoma metastatic. In contrast expression of GNGT1 in a wide variety of normal tissues including colon, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, liver, spleen, stomach, spinal cord, brain, testis, thyroid, salivary gland and nucleated blood cells was generally low (<60 rfus).

As shown in FIG. 1, the expression of GNGT1 was also low in a large variety of normal primary human cell cultures including but not limited to mammary epithelial cells, neurons, dermal fibroblasts and mesenchymal stem cells. The specificity of elevated GNGT1 expression in malignant tumors of diverse origin shown herein demonstrates that GNGT1 is a marker for the diagnosis of many types of cancers, including metastatic disease. GNGT1 may also be used as a target for therapeutic intervention in many cancers. Therapeutics that target GNGT1 can be identified using the methods described herein and therapeutics that target GNGT1 include, but are not limited to, antibodies that modulate the activity of GNGT1. The manufacture and use of antibodies are described herein.

### Example 2

### C12ORF56

C12ORF56 (Accession number NM_{—}001099676.1) encodes an uncharacterized open reading frame of unknown function. Surprisingly, it is shown herein that C12ORF56 is a novel marker for many types of malignant tumors from diverse tissues of origin, including but not limited to tumors of the uterus, rectum, cervix, ovary, lung, kidney, esophagus, bladder, testis, prostate, liver, soft tissue, cartilage, endometrium and metastatic tumors.

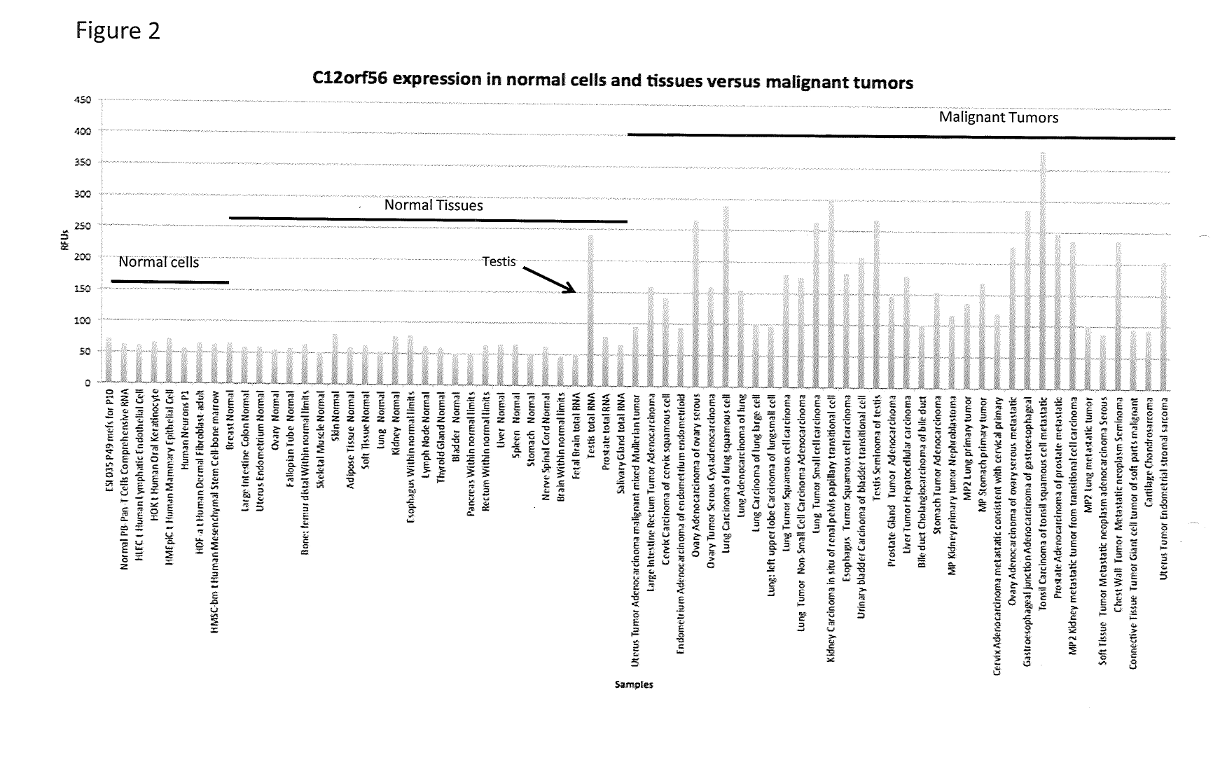

As shown in FIG. 2, C12ORF56 expression was assayed by Illumina microarray, a probe specific for C12ORF56 (probe sequence TGCCAGCCTTGCAGAAAAGGC TCCCATTGTGTTACCCCATCACTCAACCT; (SEQ ID NO: 20) Illumina probe ID ILMN_{—}1770616). The assay detected strong gene expression (>80 rfus) in uterus tumor adenocarcinoma, large intestine rectum tumor adenocarcinoma, cervix carcinoma of cervix squamous cell, endometrium adenocarcinoma of endometrium endometrioid, Ovary Adenocarcinoma of ovary serous, Ovary Tumor Serous Cystadenocarcinoma, Lung Carcinoma of lung squamous cell, Lung Adenocarcinoma of lung, Lung Carcinoma of lung large cell, Lung: left upper lobe Carcinoma of lung small cell, Lung Tumor Squamous cell carcinoma, Lung Tumor Non-Small Cell Carcinoma Adenocarcinoma, Lung Tumor Small cell carcinoma, Kidney Carcinoma in situ of renal pelvis papillary transitional cell, Esophagus Tumor Squamous cell carcinoma, Urinary bladder Carcinoma of bladder transitional cell, Testis Seminoma of testis, Prostate Gland Tumor Adenocarcinoma, Liver Tumor Hepatocellular carcinoma, Bile duct Cholangiocarcinoma of bile duct, Stomach Tumor Adenocarcinoma, Kidney primary tumor Nephroblastoma, Lung primary tumor, Stomach primary tumor, Cervix Adenocarcinoma metastatic consistent with cervical primary, Ovary Adenocarcinoma of ovary serous metastatic, Gastroesophageal junction Adenocarcinoma of gastroesophageal junction metastatic, Tonsil Carcinoma of tonsil squamous cell metastatic, Prostate Adenocarcinoma of prostate metastatic, Kidney metastatic tumor from transitional cell carcinoma, Lung metastatic tumor, Soft Tissue Tumor Metastatic neoplasm adenocarcinoma, Serous cystadenocarcinoma, Chest Wall Tumor Metastatic neoplasm, Seminoma, Connective Tissue, Tumor Giant cell tumor of soft parts malignant, Cartilage Chondrosarcoma, Uterus Tumor Endometrial stromal sarcoma.

With the exception of testis, expression of C12ORF56 in a wide variety of normal tissues including colon, cervix, endometrium, uterus myometrium, ovary, fallopian tube, bone, skeletal muscle, skin, adipose tissue, soft tissue, lung, kidney, esophagus, lymph node, thyroid, urinary bladder, pancreas, prostate, rectum, liver, spleen, stomach, spinal cord, brain, testis, thyroid, salivary gland and nucleated blood cells was generally low (<80 rfus).

As shown in FIG. 2, the expression of C12ORF56 is also low in a large variety of normal primary human cell cultures including but not limited to mammary epithelial cells, neurons, dermal fibroblasts and mesenchymal stem cells. The specificity of elevated C12ORF56 expression in malignant tumors of diverse origin shown herein demonstrates that C12ORF56 is a marker for the diagnosis of cancer, including but not limited to those described in this example.

Therapeutics that target C12ORF56 can be identified using the methods described herein and therapeutics that target C12ORF56 include, but are not limited to, antibodies that modulate the activity of C12ORF56. The manufacture and use of antibodies are described herein.

### Example 3

### COL10A1

COL10A1 (Accession number NM_{—}000493.3) encodes collagen type X alpha1, a collagen that is expressed by hypertrophic chondrocytes during endochondral ossification. Surprisingly, the data herein demonstrates COL10A1 is a novel marker for many types of malignant tumors from diverse tissues of origin, including but not limited to tumors of the kidney, cervix, endometrium, ovary, lung, pleura, bladder, pancreas, testis, colon, rectum, liver, breast, soft tissue, connective tissue, stomach, esophagus, uterus, muscle and metastatic tumors.

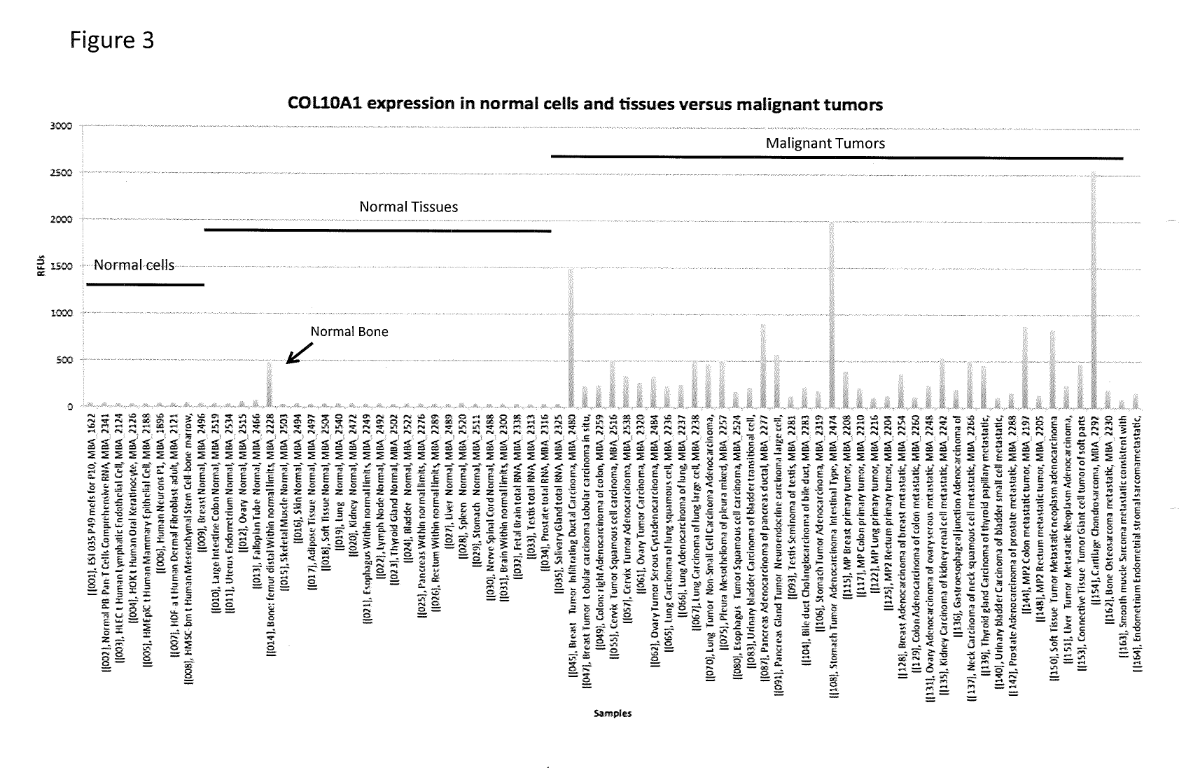

As shown in FIG. 3, GNGT1 expression is assayed by Illumina microarray, a probe specific for COL10A1 (probe sequence CCCCTAAAATATTTCTGATGGTGCACT ACTCTGAGGCCTGTATGGCCCCT; (SEQ ID NO: 21) Illumina probe ID ILMN_{—}1672776) detected strong gene expression (>100 RFUs) in Breast Tumor Infiltrating Ductal Carcinoma, Breast Tumor Lobular carcinoma, Adenocarcinoma of colon, Cervix Tumor Squamous cell carcinoma, Cervix Tumor Adenocarcinoma, Ovary Tumor Carcinoma, Ovary Tumor Serous Cystadenocarcinoma, Lung Carcinoma of lung squamous cell, Lung Adenocarcinoma of lung, Lung Carcinoma of lung large cell, Lung Tumor Non-Small Cell Carcinoma Adenocarcinoma, Pleura Mesothelioma, Esophagus Tumor Squamous cell carcinoma, Urinary bladder Carcinoma of bladder transitional cell, Pancreas Adenocarcinoma of pancreas ductal, Pancreas Gland Tumor Neuroendocrine carcinoma large cell, Testis Seminoma of testis, Bile duct Cholangiocarcinoma of bile duct, Stomach Tumor Adenocarcinoma, Stomach Tumor Adenocarcinoma Intestinal Type, Breast primary tumor, Colon primary tumor, MP Lung primary tumor, Rectum primary tumor, Breast Adenocarcinoma of breast metastatic, Colon Adenocarcinoma of colon metastatic, Ovary Adenocarcinoma of ovary serous metastatic, Kidney Carcinoma of kidney renal cell metastatic, Gastroesophageal junction Adenocarcinoma of gastroesophageal junction metastatic, Neck Carcinoma of neck squamous cell metastatic, Thyroid gland Carcinoma of thyroid papillary metastatic, Urinary bladder Carcinoma of bladder small cell metastatic, Prostate Adenocarcinoma of prostate metastatic, MP2 Colon metastatic tumor, Rectum metastatic tumor, Soft Tissue Tumor Metastatic neoplasm adenocarcinoma Serous cystadenocarcinoma, Liver Tumor Metastatic Neoplasm Adenocarcinoma, Connective Tissue Tumor Giant cell tumor of soft parts malignant, Cartilage Chondrosarcoma, Bone Osteosarcoma metastatic, Smooth muscle Sarcoma metastatic consistent with leiomyosarcoma primary, and Endometrium Endometrial stromal sarcomametastatic. In contrast, expression of COL10A1 in a wide variety of normal tissues including colon, cervix, endometrium, uterus myometrium, ovary, fallopian tube, skeletal muscle, skin, adipose tissue, soft tissue, lung, kidney, esophagus, lymph node, thyroid, urinary bladder, pancreas, prostate, rectum, liver, spleen, stomach, spinal cord, brain, testis, thyroid, salivary gland and nucleated blood cells was generally low (<60 RFUs), with the exception of normal bone.

As shown in FIG. 3, the expression of COL10A1 is also low in a large variety of normal primary human cell cultures including but not limited to mammary epithelial cells, neurons, dermal fibroblasts and mesenchymal stem cells. The specificity of elevated COL10A1 expression in malignant tumors of diverse origin shown herein demonstrates that COL10A1 is a marker for the diagnosis of many types of cancers, including metastatic disease and a target for therapeutic intervention in many cancers.

Therapeutics that target COL10A1 can be identified using the methods described herein and therapeutics that target COL10A1 include, but are not limited to, antibodies that modulate the activity of COL10A1. The manufacture and use of antibodies are described herein. For example, therapeutics can be used to modulate (e.g. inhibit) the interaction with COL1.

### Example 4

### SLC35D3

SLC35D3 (Accession number NM_{—}001008783.1) encodes SLC35D3, an orphan nucleotide sugar transporter. Surprisingly, we disclose here that SLC35D3 is a novel marker for many types of malignant tumors from diverse tissues of origin, including but not limited to tumors of the colon, rectum, liver, stomach, lung, pleura, bladder, cervix and ovary.

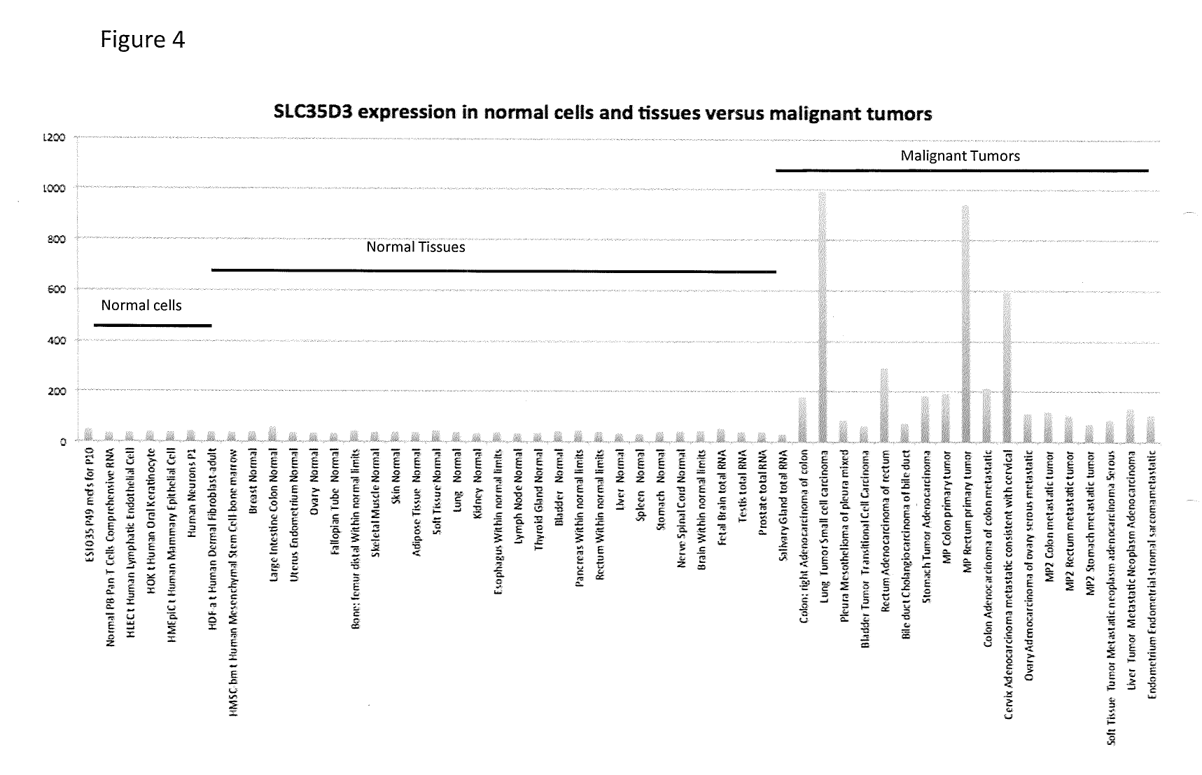

As shown in FIG. 4, SLC35D3 expression was assayed by Illumina microarray, a probe specific for SLC35D3 (probe sequence ACTGAAACCCAGCCAGAAGAG GGACCACCTGTAAAGCAAGTCCTTTCAAG; (SEQ ID NO: 22) Illumina probe ID ILMN_{—}1702419) detected strong gene expression (>70 RFUs) in Adenocarcinoma of colon, Lung Tumor Small cell carcinoma, Pleura Mesothelioma of pleura mixed, Bladder Tumor Transitional Cell Carcinoma, Rectum Adenocarcinoma of rectum, Bile duct Cholangiocarcinoma of bile duct, Stomach Tumor Adenocarcinoma, Colon primary tumor, Rectum primary tumor, Colon Adenocarcinoma of colon metastatic, Cervix Adenocarcinoma metastatic consistent with cervical primary, Ovary Adenocarcinoma of ovary serous metastatic, Colon metastatic tumor, Rectum metastatic tumor, Stomach metastatic tumor, Soft Tissue Tumor Metastatic neoplasm adenocarcinoma Serous cystadenocarcinoma, Liver Tumor Metastatic Neoplasm Adenocarcinoma, Endometrium Endometrial stromal sarcomametastatic. In expression of SLC35D3 in a wide variety of normal tissues including colon, cervix, endometrium, uterus myometrium, ovary, fallopian tube, skeletal muscle, skin, adipose tissue, soft tissue, lung, kidney, esophagus, lymph node, thyroid, urinary bladder, pancreas, prostate, rectum, liver, spleen, stomach, spinal cord, brain, bone, testis, thyroid, gland and nucleated blood cells was generally low (<60 RFUs).

As shown in FIG. 4, the expression of SLC35D3 is also low in a large variety of normal primary human cell cultures including but not limited to mammary epithelial cells, neurons, dermal fibroblasts and mesenchymal stem cells. The specificity of elevated SLC35D3 expression in malignant tumors of diverse origin shown herein demonstrates that SLC35D3 is a marker for the diagnosis of many types of cancers (e.g. including, but not limited to, those described in this example), including metastatic disease and a target for therapeutic intervention in many cancers.

Therapeutics that target SLC35D3 can be identified using the methods described herein and therapeutics that target SLC35D3 include, but are not limited to, antibodies that modulate the activity of SLC35D3. The manufacture and use of antibodies are described herein. For example, therapeutics can be used to modulate (e.g. inhibit or enhance) the transport function of SLC35D3. Assays can also be used to identify new therapeutics that can inhibit the function of SLC35D3. Any transport assay can be used to identify the new inhibitors.

### Example 5

### snaR-A

SNAR-A (Accession number BU536065) encodes SNAR-A, a small, untranslated RNA of unknown function. While snaR-A was shown to be up-regulated in cellular immortalization in vitro (Parrott, et al, NAR 2011, Vol 39, No. 4), its expression in cancer in vivo has not been reported. Surprisingly, it is disclosed herein that SNAR-A is a novel marker for many types of malignant tumors from diverse tissues of origin, including but not limited to tumors of the kidney, cervix, endometrium, ovary, lung, pleura, bladder, pancreas, testis, colon, rectum, liver, breast, soft tissue, connective tissue, stomach, esophagus, prostate, bone, uterus, muscle and metastatic tumors.

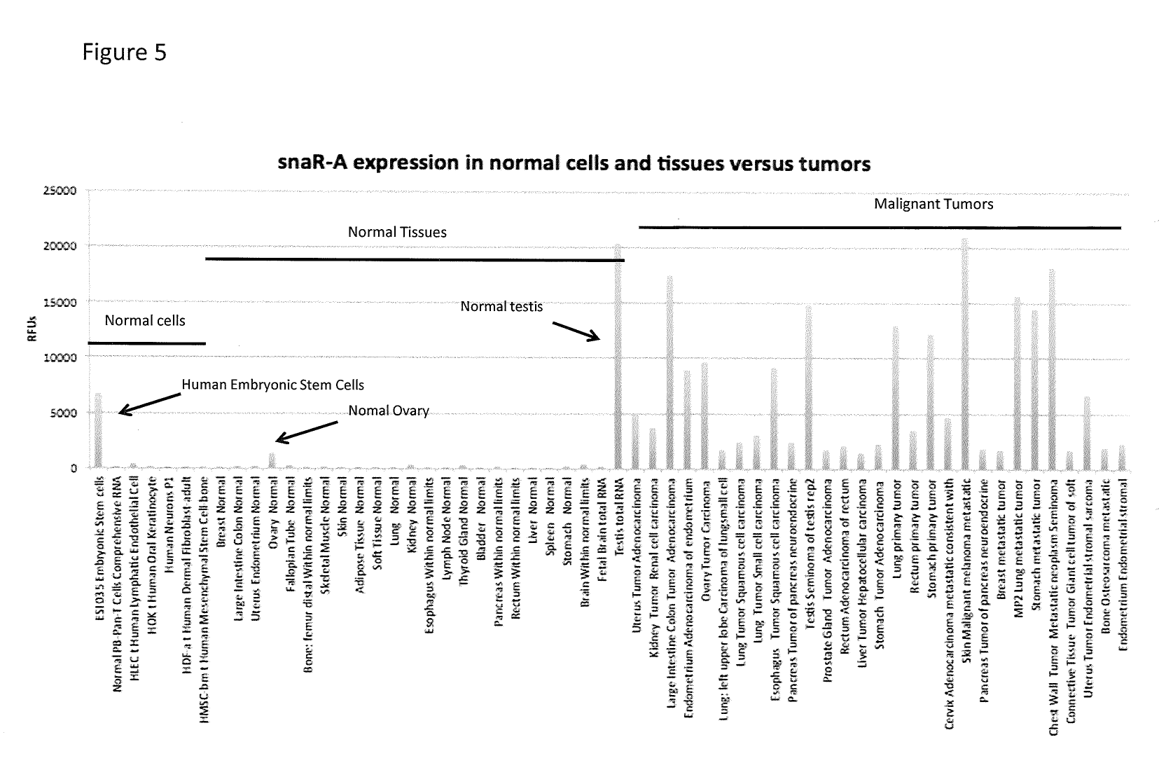

As shown in FIG. 5, SNAR-A expression was assayed by Illumina microarray, a probe specific for SNAR-A (probe sequence TTCCAGGGCACGAGTTCGAGG CCAGCCTGGTCCACATGGGTCGGaaaaaa; (SEQ ID NO: 23) Illumina probe ID ILMN_{—}1881909) detected strong gene expression (>1500 RFUs) in Uterus Tumor Adenocarcinoma, Kidney Tumor Renal cell carcinoma, Large Intestine Colon Tumor Adenocarcinoma, Endometrium Adenocarcinoma, Ovary Tumor, Lung tumor small cell, Lung Tumor Squamous cell carcinoma, Esophagus Tumor Squamous cell carcinoma, Pancreas Tumor of pancreas neuroendocrine, Testis Seminoma, Prostate Gland Tumor Adenocarcinoma, Rectum Adenocarcinoma of rectum, Liver Tumor Hepatocellular carcinoma, Stomach Tumor Adenocarcinoma, Lung primary tumor, Rectum primary tumor, Stomach primary tumor, Cervix Adenocarcinoma metastatic consistent with cervical primary, Skin Malignant melanoma metastatic, Pancreas Tumor of pancreas neuroendocrine metastatic, Breast metastatic tumor, Lung metastatic tumor, Stomach metastatic tumor, Chest Wall Tumor Metastatic neoplasm Seminoma, Connective Tissue Tumor Giant cell tumor of soft parts malignant, Uterus Tumor Endometrial stromal sarcoma, Bone Osteosarcoma metastatic, Endometrium Endometrial stromal sarcomametastatic. In contrast expression of SNAR-A in a wide variety of normal tissues including colon, cervix, endometrium, uterus myometrium, fallopian tube, skeletal muscle, skin, adipose tissue, soft tissue, lung, kidney, esophagus, lymph node, thyroid, urinary bladder, pancreas, prostate, rectum, liver, spleen, stomach, spinal cord, brain, bone, thyroid, salivary gland and nucleated blood cells was generally low (<500 RFUs), with the exception of reproductive tissues, ovary and testis.

As shown in FIG. 5, the expression of SNAR-A is also low in a large variety of normal primary human cell cultures including but not limited to neurons, dermal fibroblasts and mesenchymal stem cells. The specificity of elevated SNAR-A expression in malignant tumors of diverse origin shown herein demonstrates that SNAR-A is a marker for the diagnosis of many types of cancers (e.g. including but not limited to the cancers described in this example), including metastatic disease and a target for therapeutic intervention in many cancers. SNAR-A can be used as a diagnostic marker of cancer in general or the specific types of cancers described herein.

Therapeutics that target SNAR-A can be identified using the methods described herein and therapeutics that target SNAR-A include, but are not limited to, antibodies that modulate the activity of SNAR-A. The manufacture and use of antibodies are described herein.

### Example 6

### SBK1

SBK1 (Accession number NM_{—}001024401.2) encodes SH3-binding domain kinase 1. Surprisingly, it is disclosed herein that SBK1 is a novel marker for many types of malignant tumors from diverse tissues of origin, including but not limited to tumors of the lymph node. kidney, cervix, endometrium, ovary, lung, pleura, bladder, pancreas, testis, colon, rectum, liver, breast, soft tissue, bladder, brain, tonsil, thyroid, connective tissue, stomach, esophagus, prostate, bone, uterus, muscle and metastatic tumors.

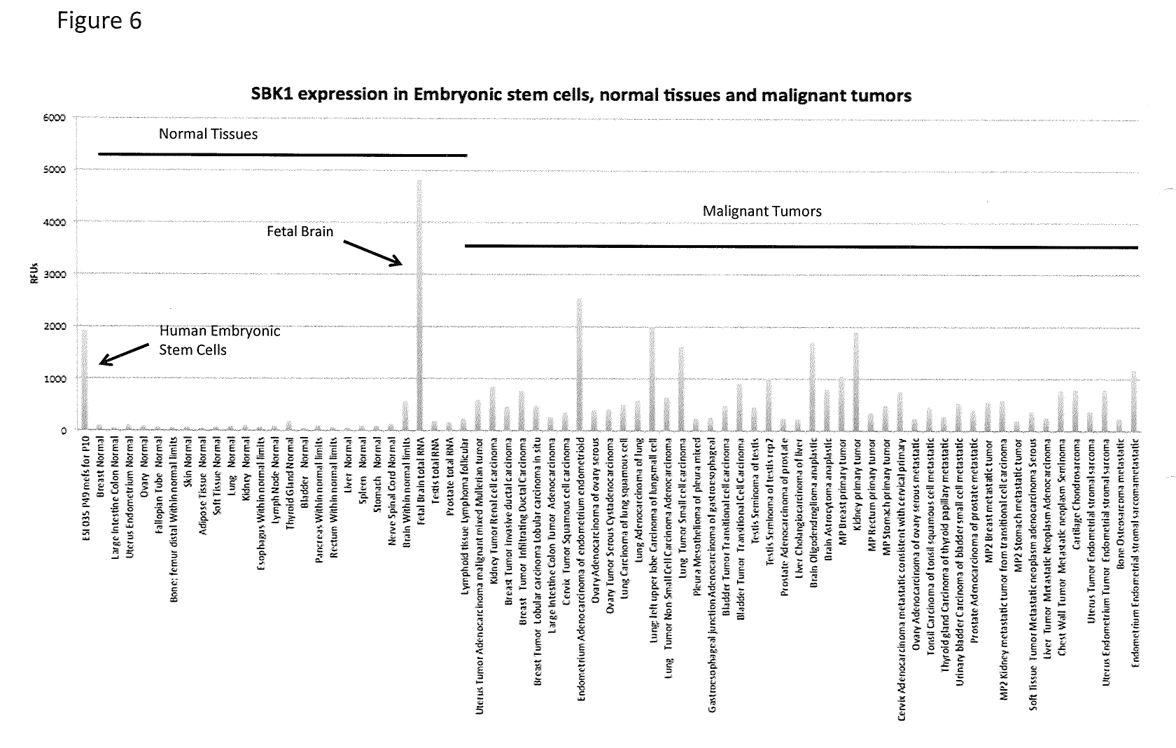

As shown in FIG. 6, SBK1 expression was assayed by Illumina microarray, a probe specific for SBK1 (probe sequence CAGAGCCCCAGCCCCTCATGTCTTGCCGCCCTT CCTCCATGTGTTTGTAA; (SEQ ID NO: 24) Illumina probe ID ILMN_{—}1728298) detected strong gene expression (>240 RFUs) in Lymphoma follicular, Uterus Tumor Adenocarcinoma malignant tumor, Kidney Tumor Renal cell carcinoma, Breast Tumor invasive ductal carcinoma, Breast Tumor Infiltrating Ductal Carcinoma, Breast Tumor Lobular carcinoma Lobular carcinoma in situ, Large Intestine Colon Tumor Adenocarcinoma, Cervix Tumor Squamous cell carcinoma, Endometrium Adenocarcinoma of endometrium endometrioid, Ovary Adenocarcinoma of ovary serous, Ovary Tumor Serous Cystadenocarcinoma, Lung Carcinoma of lung squamous cell, Lung Adenocarcinoma of lung, Lung: left upper lobe Carcinoma of lung small cell, Lung Tumor Non-Small Cell Carcinoma Adenocarcinoma, Lung Tumor Small cell carcinoma, Pleura Mesothelioma of pleura mixed, Gastroesophageal junction Adenocarcinoma of gastroesophageal junction, Bladder Tumor Transitional cell carcinoma, Bladder Tumor Transitional Cell Carcinoma, Testis Seminoma of testis, Testis Seminoma, Prostate Adenocarcinoma of prostate, Liver Cholangiocarcinoma of liver, Brain Oligodendroglioma anaplastic, Brain Astrocytoma anaplastic, Breast primary tumor, Kidney primary tumor, Rectum primary tumor, Stomach primary tumor, Cervix Adenocarcinoma metastatic consistent with cervical primary, Ovary Adenocarcinoma of ovary serous metastatic, Tonsil Carcinoma of tonsil squamous cell metastatic, Thyroid gland Carcinoma of thyroid papillary metastatic, Urinary bladder Carcinoma of bladder small cell metastatic, Prostate Adenocarcinoma of prostate metastatic, Breast metastatic tumor, Kidney metastatic tumor from transitional cell carcinoma, Stomach metastatic tumor, Soft Tissue Tumor Metastatic neoplasm adenocarcinoma Serous cystadenocarcinoma, Liver Tumor Metastatic Neoplasm Adenocarcinoma, Chest Wall Tumor Metastatic neoplasm Seminoma, Cartilage Chondrosarcoma, Uterus Tumor Endometrial stromal sarcoma, Uterus Endometrium Tumor Endometrial stromal sarcoma, Bone Osteosarcoma metastatic, Endometrium Endometrial stromal sarcomametastatic. In contrast, expression of SBK1 in a wide variety of normal tissues including colon, cervix, endometrium, uterus myometrium, fallopian tube, skeletal muscle, skin, adipose tissue, soft tissue, lung, kidney, esophagus, lymph node, thyroid, urinary bladder, pancreas, prostate, rectum, liver, spleen, stomach, spinal cord, bone, thyroid, and salivary gland was generally low (<500 RFUs), with the exception of fetal brain.

The specificity of elevated SBK1 expression in malignant tumors of diverse origin shown herein demonstrates that SBK1 is a marker for the diagnosis of many types of cancers (e.g. including but not limited to the cancers described in this example), including metastatic disease and a target for therapeutic intervention in many cancers.

Therapeutics that target SBK1 can be identified using the methods described herein and therapeutics that target SBK1 include, but are not limited to, antibodies that modulate the activity of SBK1. The manufacture and use of antibodies are described herein.

### Example 7

### DSCR8

DSCR8 (Accession number NM_{—}203428.1) encodes Down Syndrome critical region 8. Surprisingly, it is disclosed here that DSCR8 is a novel marker for many types of malignant tumors from diverse tissues of origin, including but not limited to tumors of the endometrium, ovary, lung, bladder, testis, bladder, stomach, esophagus, skin melanomas and metastatic tumors.

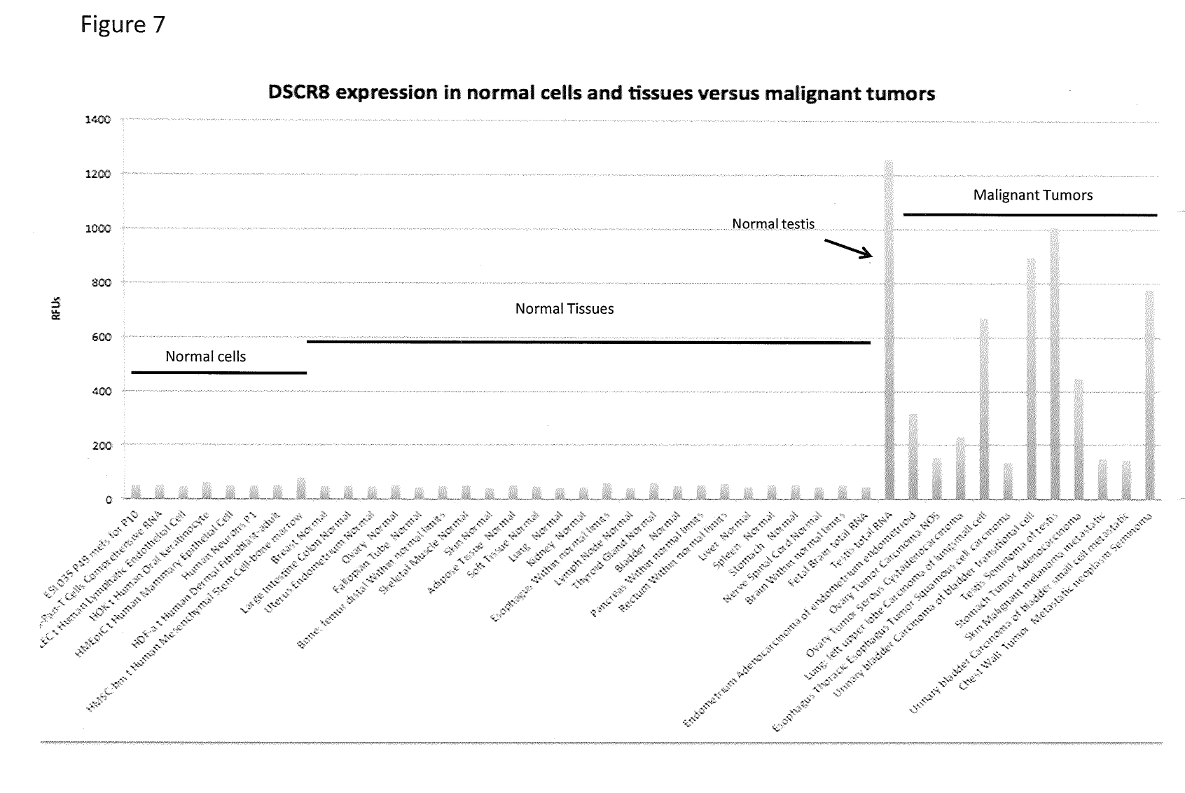

As shown in FIG. 7, DSCR8 expression was assayed by Illumina microarray, a probe specific for DSCR8 (probe sequence TCCCACTTGGCAGGGGCCGTCTTGTCCACTC GTTTCTGTAAACATGGGTG; (SEQ ID NO:25) Illumina probe ID ILMN_{—}1763901) detected strong gene expression (>145 RFUs) in Endometrium Adenocarcinoma, Ovary Tumor Carcinoma, Ovary Tumor Serous Cystadenocarcinoma, Carcinoma of lung small cell, Esophagus Tumor Squamous cell carcinoma, Urinary bladder Carcinoma transitional cell, Seminoma of testis, Stomach Tumor Adenocarcinoma, Skin Malignant melanoma metastatic, Urinary bladder Carcinoma of bladder small cell metastatic, Chest Wall Tumor Metastatic neoplasm Seminoma. In contrast, expression of DSCR8 in a wide variety of normal tissues including colon, cervix, endometrium, uterus myometrium, fallopian tube, skeletal muscle, skin, adipose tissue, soft tissue, lung, kidney, esophagus, lymph node, thyroid, urinary bladder, pancreas, prostate, rectum, liver, spleen, stomach, spinal cord, bone, thyroid, and salivary gland was generally low (<80 RFUs), with the exception of testis.

The specificity of elevated DSCR8 expression in malignant tumors of diverse origin shown herein demonstrates that DSCR8 is a marker for the diagnosis of many types of cancers (e.g. including but not limited to the cancers described in this example), including metastatic disease and a target for therapeutic intervention in many cancers.

Therapeutics that target DSCR8 can be identified using the methods described herein and therapeutics that target DSCR8 include, but are not limited to, antibodies that modulate the activity of DSCR8. The manufacture and use of antibodies are described herein.

### Example 8

### CELSR3

CELSR3 (Accession number NM 001407.2) encodes cadherin, EGF LAG seven-pass G-type receptor 3. Surprisingly, it is disclosed here that CELSR3 is a novel marker for many types of malignant tumors from diverse tissues of origin, including but not limited to tumors of the lymph node. kidney, cervix, endometrium, ovary, lung, pleura, bladder, pancreas, testis, colon, rectum, liver, breast, soft tissue, bladder, brain, tonsil, thyroid, connective tissue, stomach, esophagus, prostate, bone, uterus, testis, muscle and metastatic tumor.

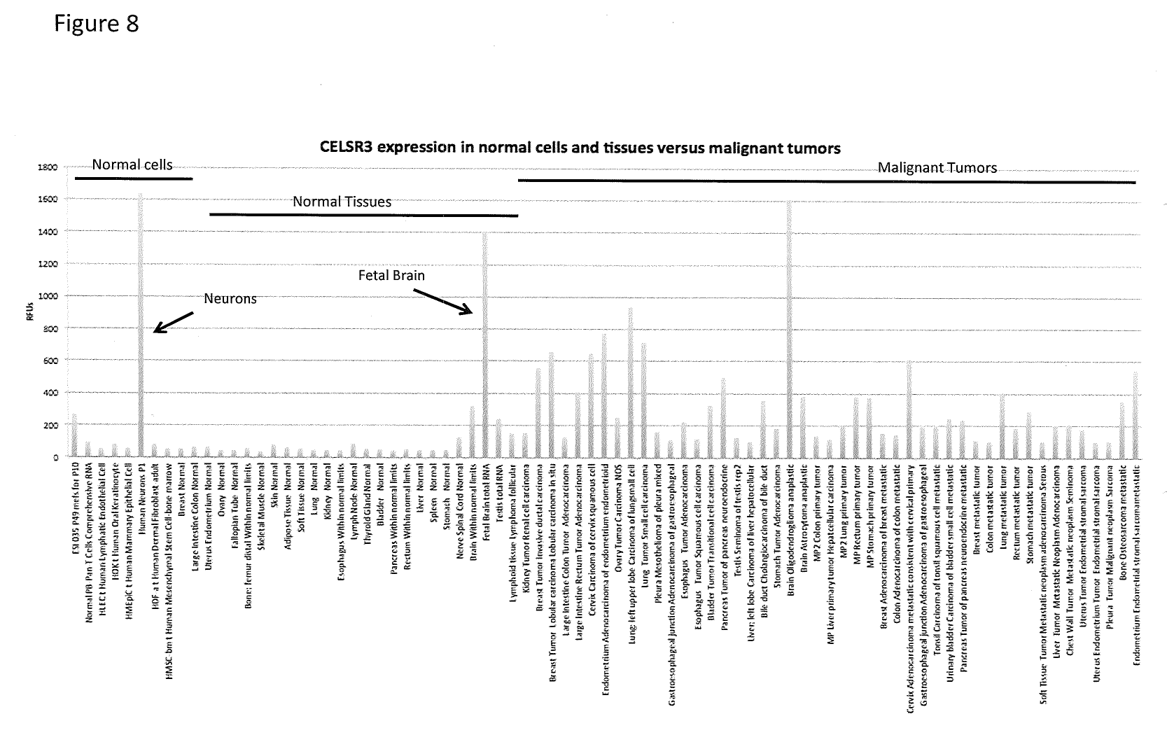

As shown in FIG. 8, CELSR3 expression was assayed by Illumina microarray, a probe specific for CELSR3 (probe sequence CCCAGCGGCCCTCTTTCCTGTCTGTGTAAAT TGTTCCGTGAAGCCGCGCT; (SEQ ID NO: 26) Illumina probe ID ILMN_{—}1691290) detected strong gene expression (>100 RFUs) in Lymphoma follicular, Kidney Tumor Renal cell carcinoma, Breast Tumor invasive ductal carcinoma, Breast Tumor Lobular carcinoma, Large Intestine Colon Tumor Adenocarcinoma, Large Intestine Rectum Tumor Adenocarcinoma, Cervix Carcinoma of cervix squamous cell, Endometrium Adenocarcinoma, Ovary Tumor Carcinoma, Carcinoma of lung small cell, Lung Tumor Small cell carcinoma, Pleura Mesothelioma of pleura mixed, Gastroesophageal junction Adenocarcinoma of gastroesophageal junction, Esophagus Tumor Adenocarcinoma, Esophagus Tumor Squamous cell carcinoma, Bladder Tumor Transitional cell carcinoma, Pancreas Tumor of pancreas neuroendocrine, Testis Seminoma, Liver: left lobe Carcinoma of liver hepatocellular, Bile duct Cholangiocarcinoma of bile duct, Stomach Tumor Adenocarcinoma, Brain Oligodendroglioma anaplastic, Brain Astrocytoma anaplastic, Colon primary tumor, Liver primary tumor Hepatcellular carcinoma, Lung primary tumor, Rectum primary tumor, Stomach primary tumor, Breast Adenocarcinoma of breast metastatic, Colon Adenocarcinoma of colon metastatic, Cervix Adenocarcinoma metastatic consistent with cervical primary, Gastroesophageal junction Adenocarcinoma of gastroesophageal junction metastatic, Tonsil Carcinoma of tonsil squamous cell metastatic, Urinary bladder Carcinoma of bladder small cell metastatic, Pancreas Tumor of pancreas neuroendocrine metastatic, Breast metastatic tumor, Colon metastatic tumor, Lung metastatic tumor, Rectum metastatic tumor, Stomach metastatic tumor, Soft Tissue Tumor Metastatic neoplasm adenocarcinoma Serous cystadenocarcinoma, Liver Tumor Metastatic Neoplasm Adenocarcinoma, Chest Wall Tumor Metastatic neoplasm Seminoma, Uterus Tumor Endometrial stromal sarcoma, Uterus Endometrium Tumor Endometrial stromal sarcoma, Pleura Tumor Malignant neoplasm Sarcoma, Bone Osteosarcoma metastatic, Endometrium Endometrial stromal sarcomametastatic. In contrast expression of CELSR3 in a wide variety of normal tissues including colon, cervix, endometrium, uterus myometrium, fallopian tube, skeletal muscle, skin, adipose tissue, soft tissue, lung, kidney, esophagus, lymph node, thyroid, urinary bladder, pancreas, prostate, rectum, liver, spleen, stomach, bone, thyroid, and salivary gland was generally low (<95 RFUs), with the exception of fetal brain and spinal cord.

The specificity of elevated CELSR3 expression in malignant tumors of diverse origin shown herein demonstrates that CELSR3 is a marker for the diagnosis of many types of cancers (e.g. including but not limited to the cancers described in this example), including metastatic disease and a target for therapeutic intervention in many cancers.

Therapeutics that target CELSR3 can be identified using the methods described herein and therapeutics that target CELSR3 include, but are not limited to, antibodies that modulate the activity of CELSR3. The manufacture and use of antibodies are described herein.

### Example 9

### PPEF1

PPEF1 (Accession number NM_{—}152224.1) encodes protein phosphatase, EF-hand calcium binding domain 1. Surprisingly, it is disclosed here that PPEF1 is a novel marker for many types of malignant tumors from diverse tissues of origin, including but not limited to tumors of the breast, bladder, pancreas, connective tissue, cartilage, skin, bone, smooth muscle and metastatic tumors.

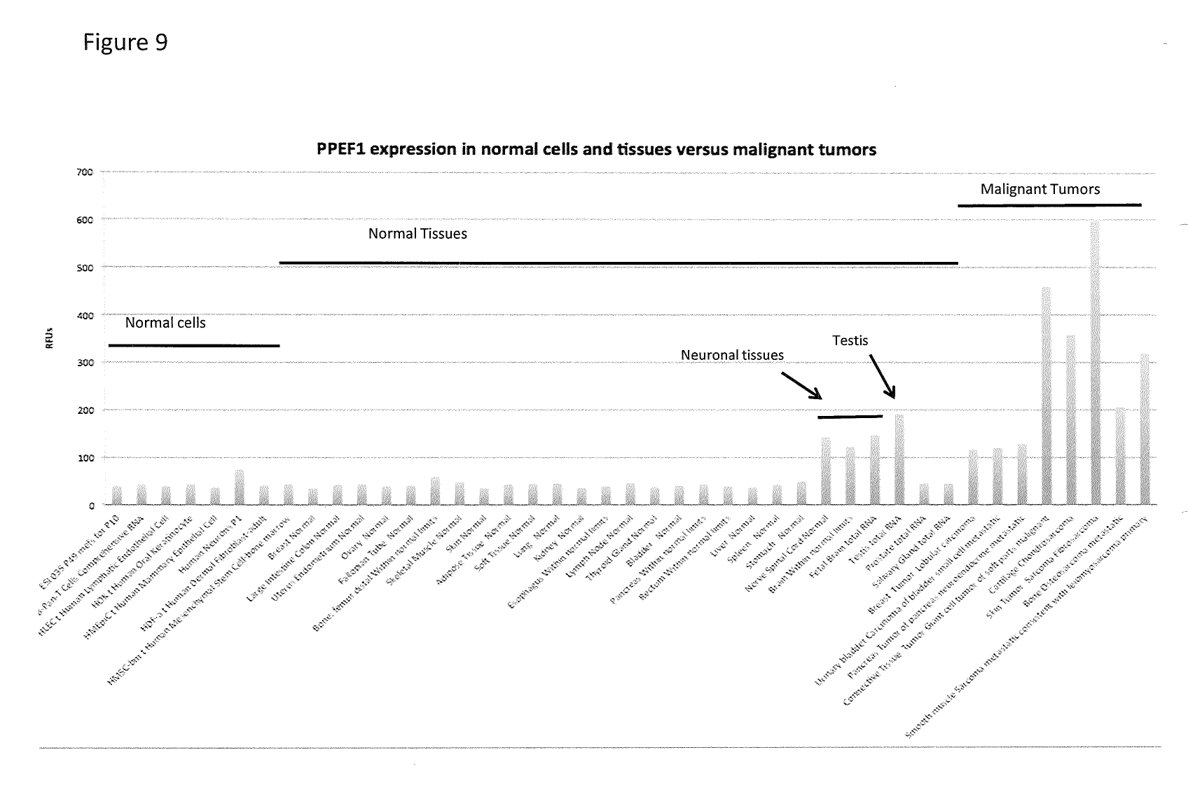

As shown in FIG. 9, PPEF1 expression was assayed by Illumina microarray, a probe specific for PPEF1 (probe sequence TGGGTTGGACCTAGTGGTGTTGTCGTGAGTGC CACCTAACCAGGAGGCCA; (SEQ ID NO: 27) Illumina probe ID ILMN_{—}1652017) detected strong gene expression (>100 RFUs) in Breast Tumor Lobular carcinoma, Carcinoma of urinary bladder small cell metastatic, Pancreas Tumor of pancreas neuroendocrine metastatic, Connective Tissue Tumor Giant cell tumor of soft parts malignant, Cartilage Chondrosarcoma, Skin Tumor Sarcoma Fibrosarcoma, Bone Osteosarcoma metastatic and Smooth muscle Sarcoma metastatic. In contrast, expression of PPEF1 in a wide variety of normal tissues including colon, cervix, endometrium, uterus myometrium, fallopian tube, skeletal muscle, skin, adipose tissue, soft tissue, lung, kidney, esophagus, lymph node, thyroid, urinary bladder, pancreas, prostate, rectum, liver, spleen, stomach, bone, thyroid, and salivary gland was generally low (<80 RFUs), with the exception of testis and neuronal tissues such as brain and spinal chord.

The specificity of elevated PPEF1 expression in malignant tumors of diverse origin shown herein demonstrates that PPEF1 is a marker for the diagnosis of many types of cancers (e.g. including but not limited to the cancers described in this example), including metastatic disease and a target for therapeutic intervention in many cancers.

Therapeutics that target PPEF1 can be identified using the methods described herein and therapeutics that target PPEF1 include, but are not limited to, antibodies that modulate the activity of PPEF1. The manufacture and use of antibodies are described in this disclosure.

### Example 10

Levels of the following proteins COL10A1, CXCL10, EPYC, IL8, LAMC2, PI3, MMP7, MMP11, MMP12, NMU, OLFM4, and WNT10A were assayed in serum using a USCN ELISA kit (USCN) according to the manufacturer's instructions. Samples came from cancer patients as well as patients who were cancer free (normal samples) In brief, 100 μL of the blank, standards, and samples with specified dilutions were added to the appropriate wells of a 96 well plate followed by 2 hours of incubation at 37° C. After removal of the liquid, 100 ul of Detection Reagent A was added to each well and incubated for 1 hour at 37° C. After removal of Reagent A, each well was washed 3 times with 350 uL of wash solution. 100 uL of Detection Reagent B was added to each well and then incubated for 30 minutes at 37° C. After removal of Reagent B, each well was washed 5 times with 350 uL of wash solution. 90 uL of Substrate solution was added to each well and incubated for 15-25 minutes at 37° C. 50 uL of Stop Solution was added to each well. The plate was read either on the Molecular Devices SpectraMax250 or the BioTek Synergy H1 plate reader at 450 nm. A standard curve was derived from the standards supplied in the kit and the sample values were extrapolated from this curve.

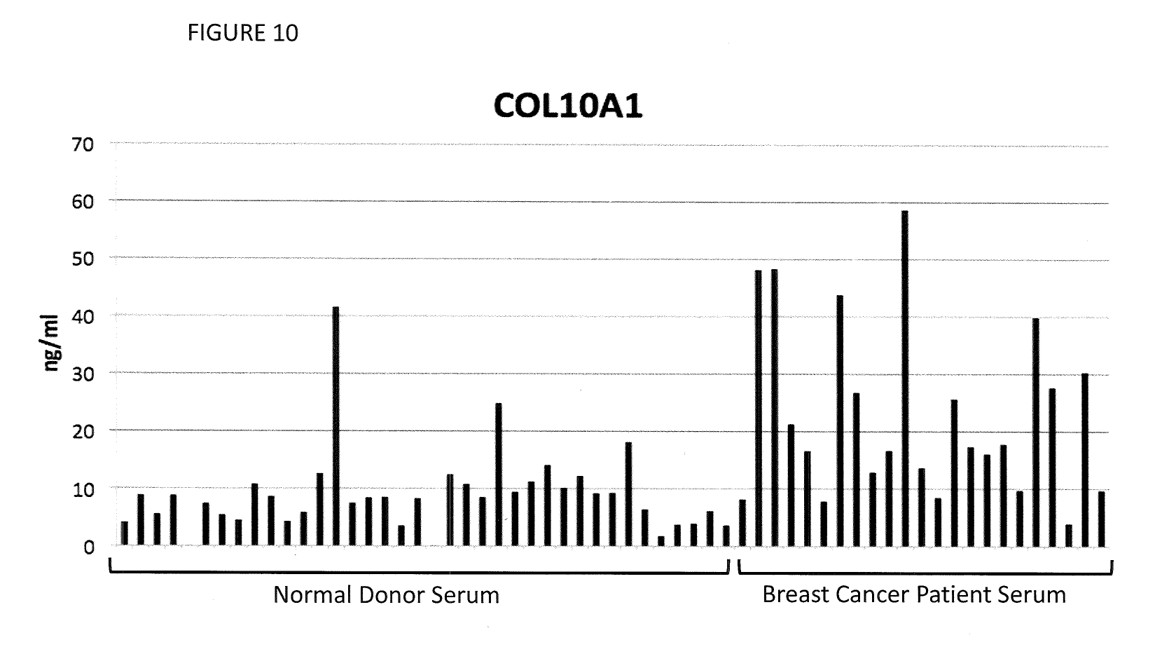

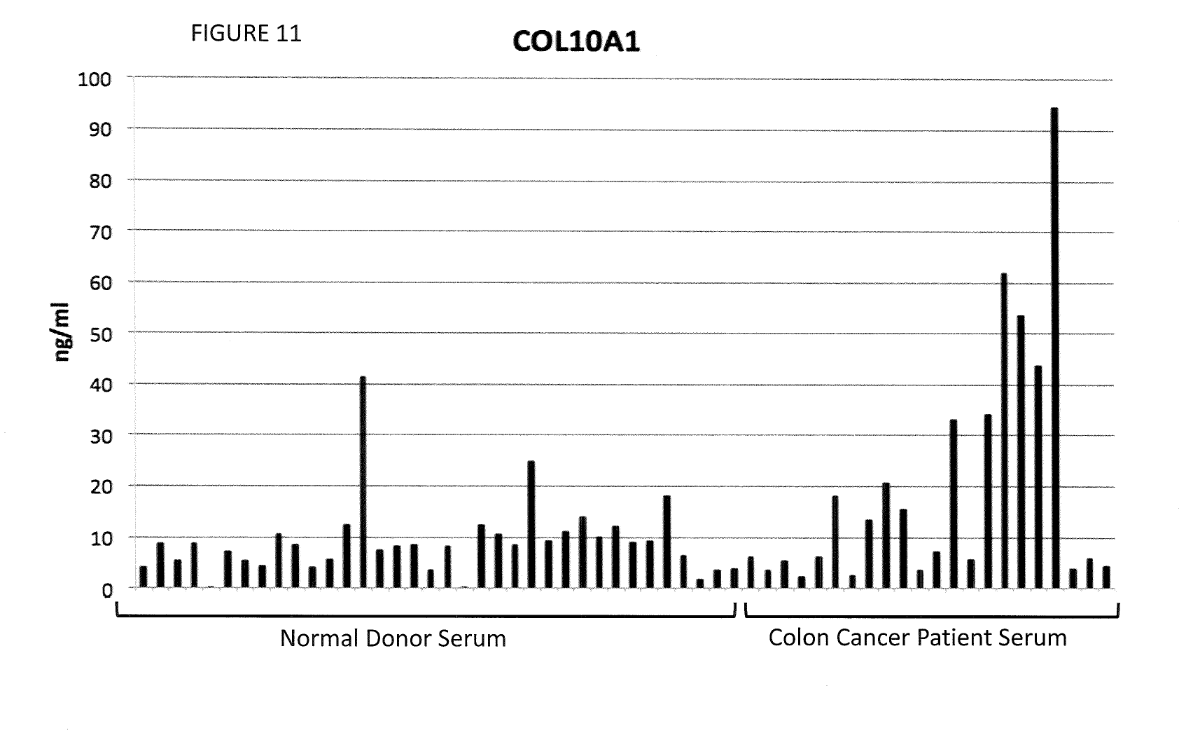

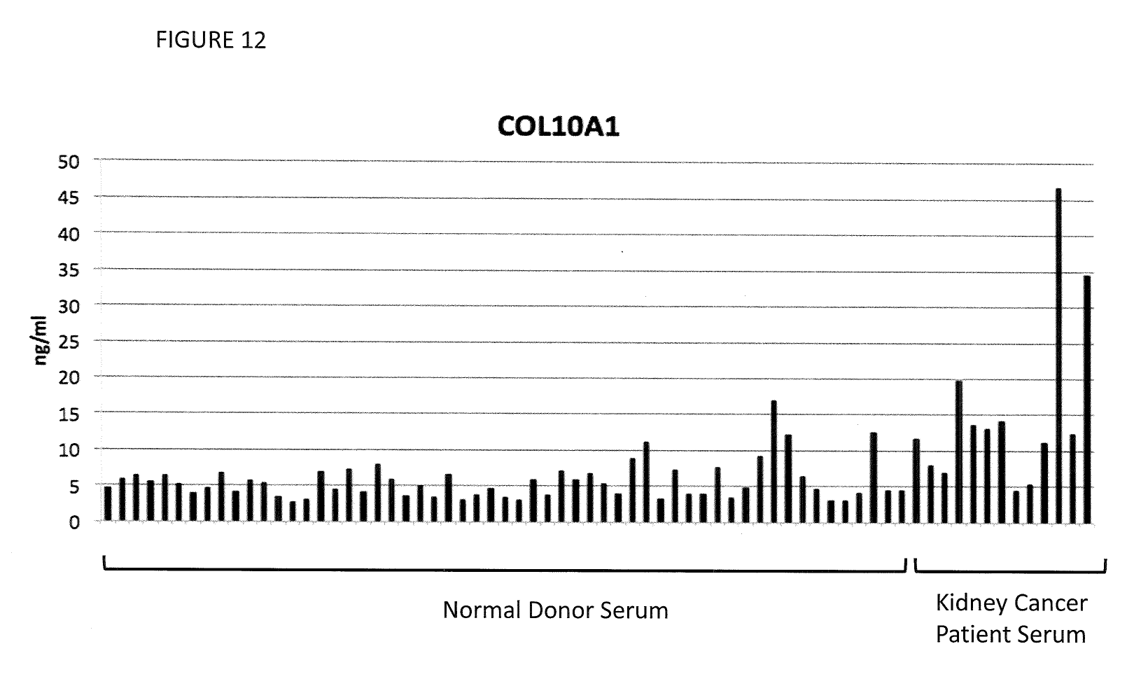

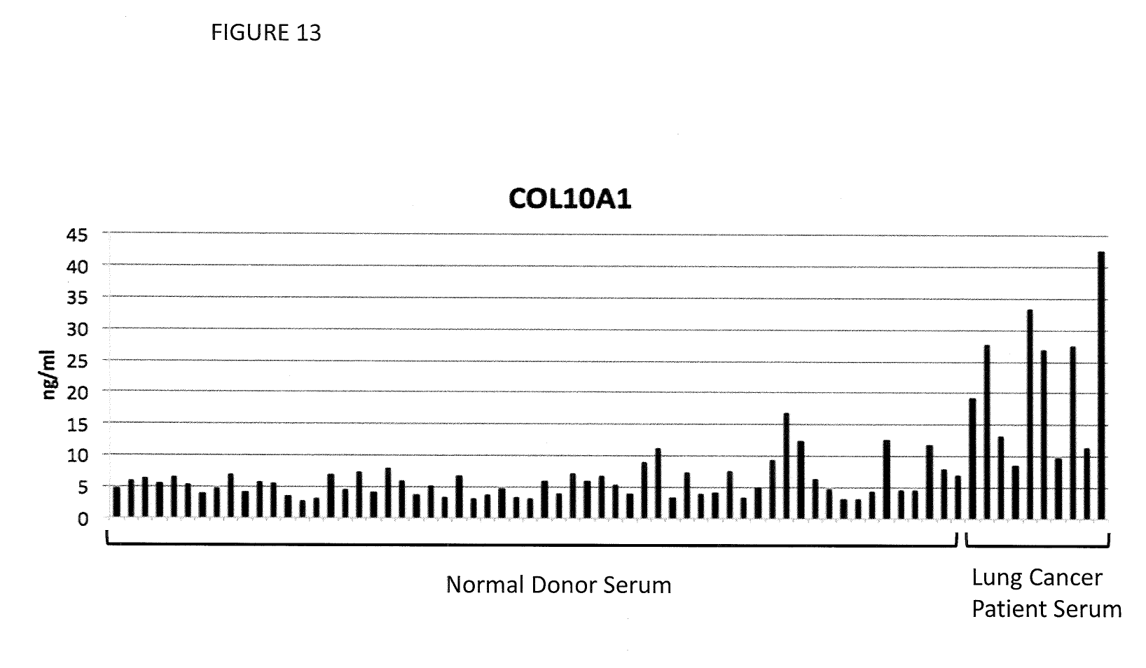

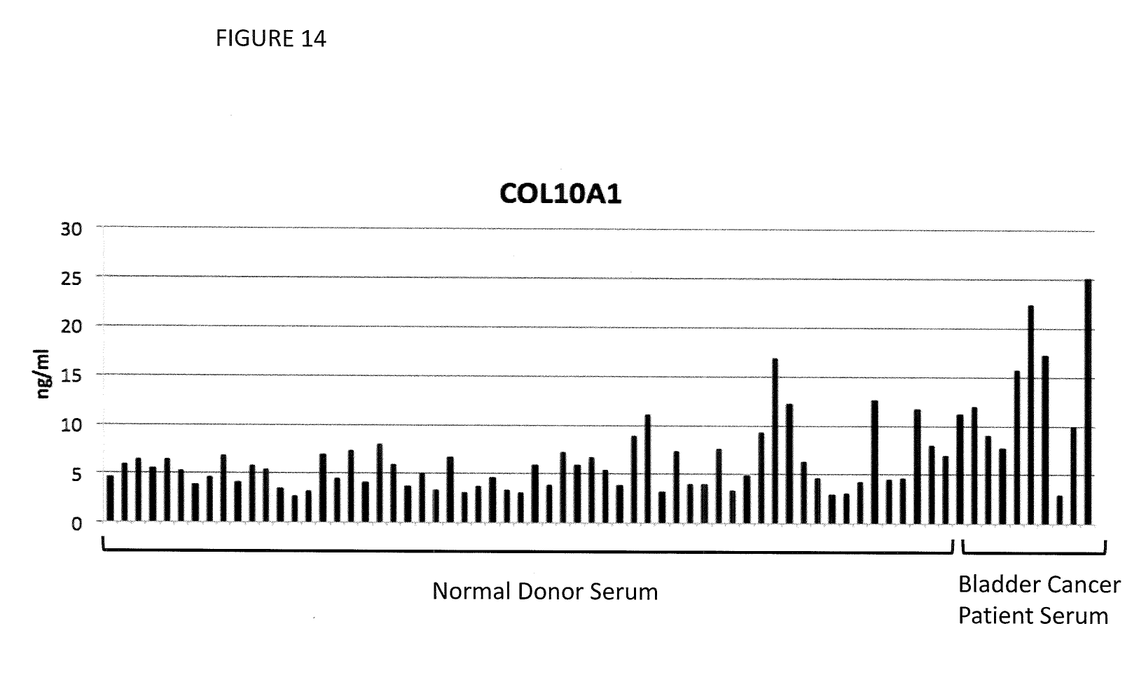

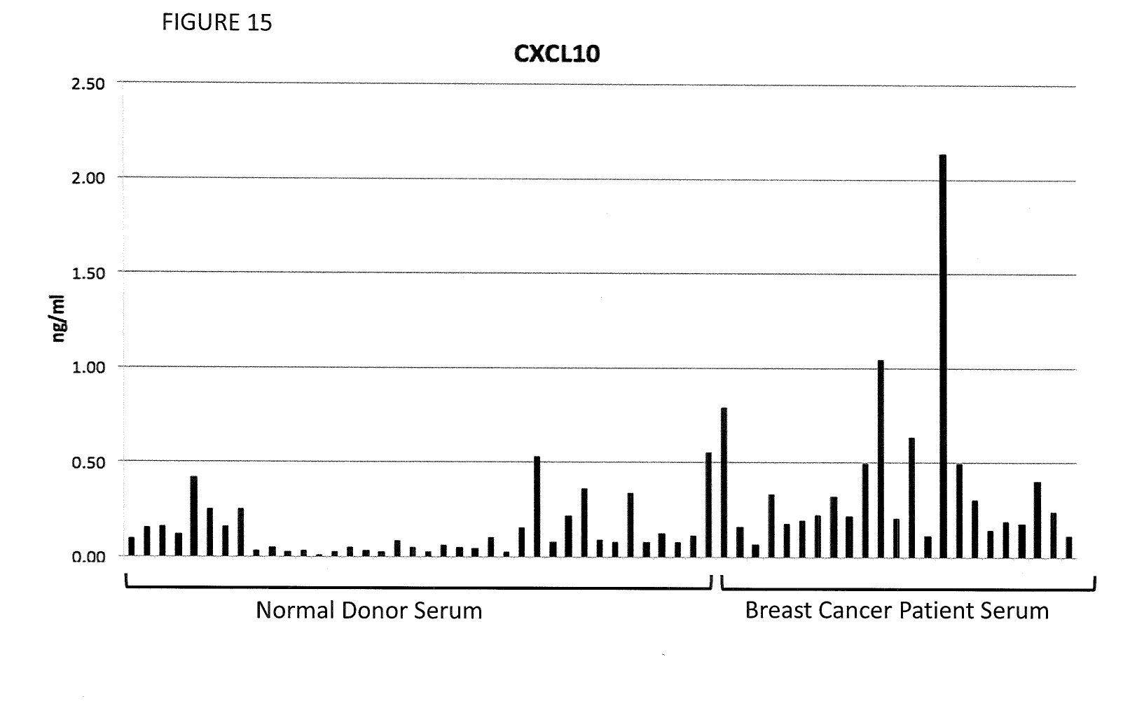

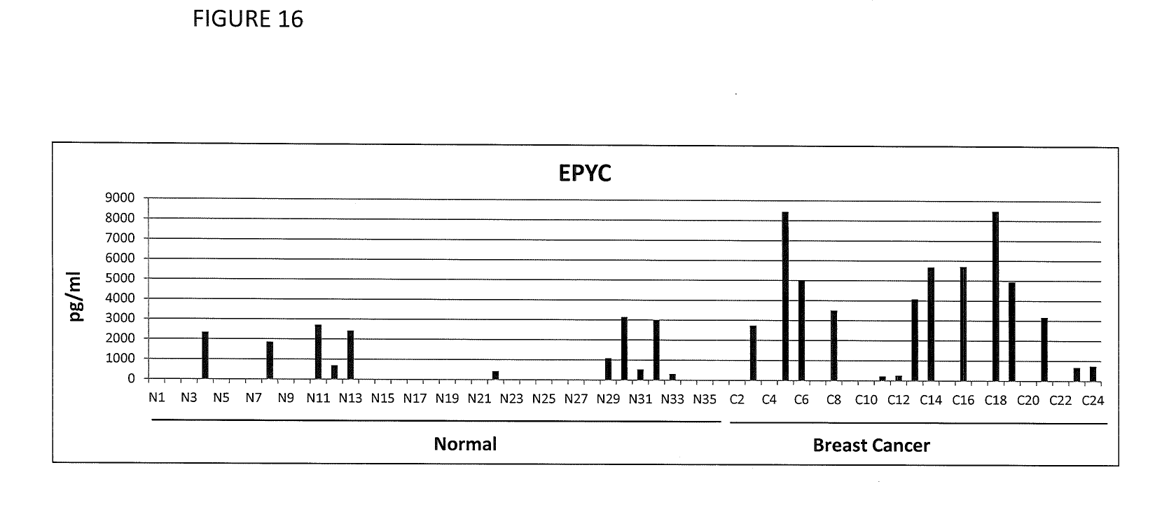

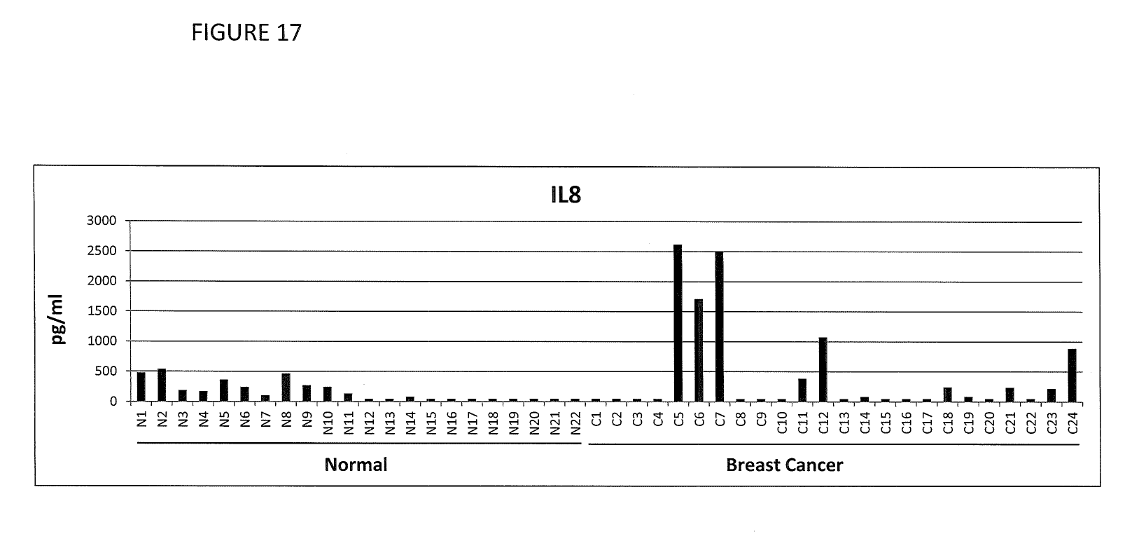

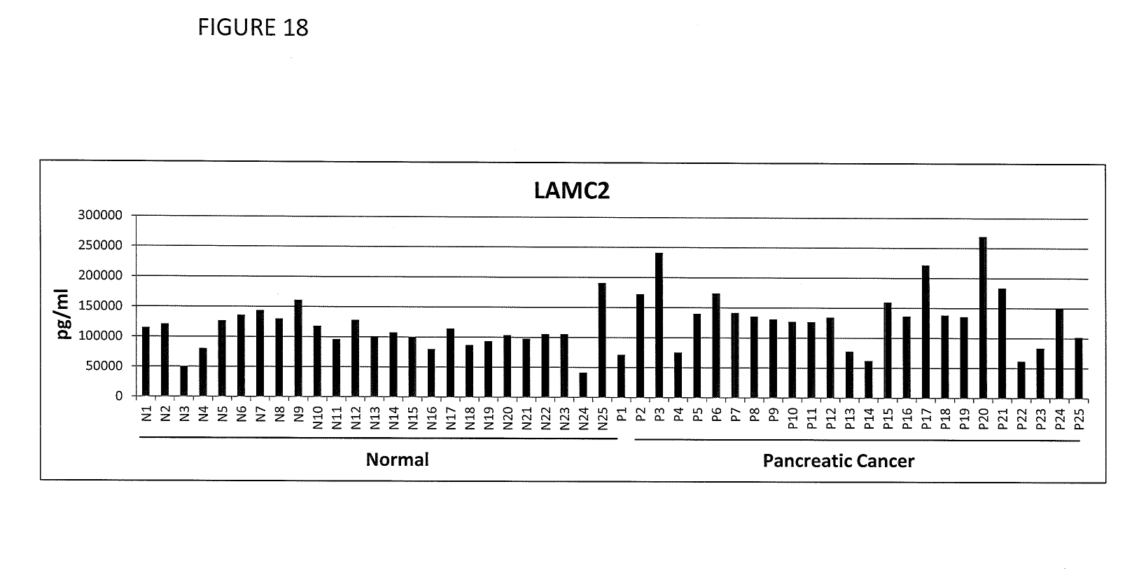

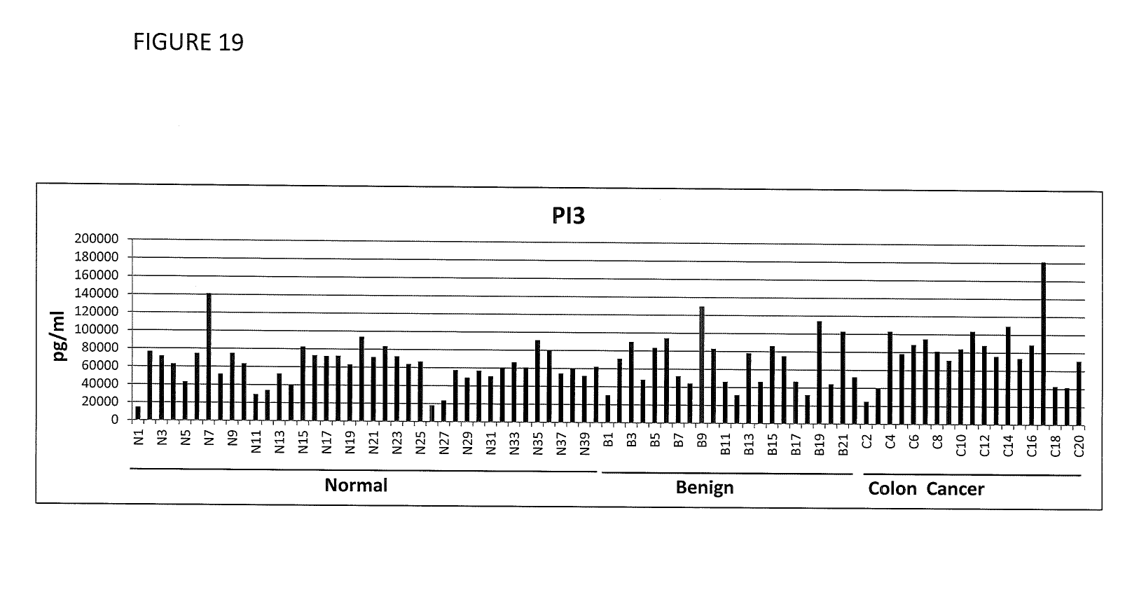

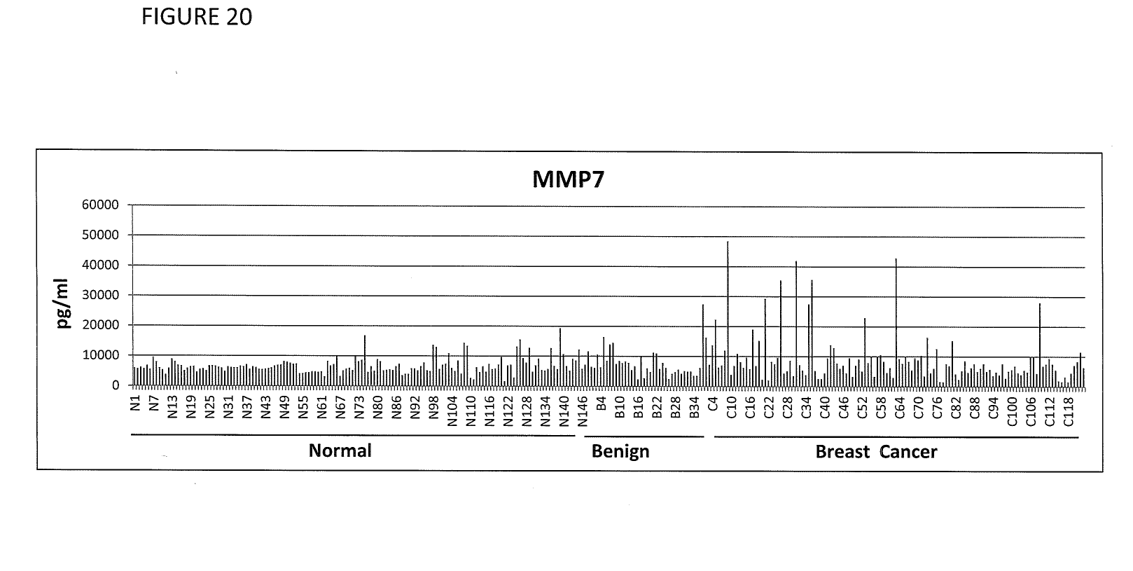

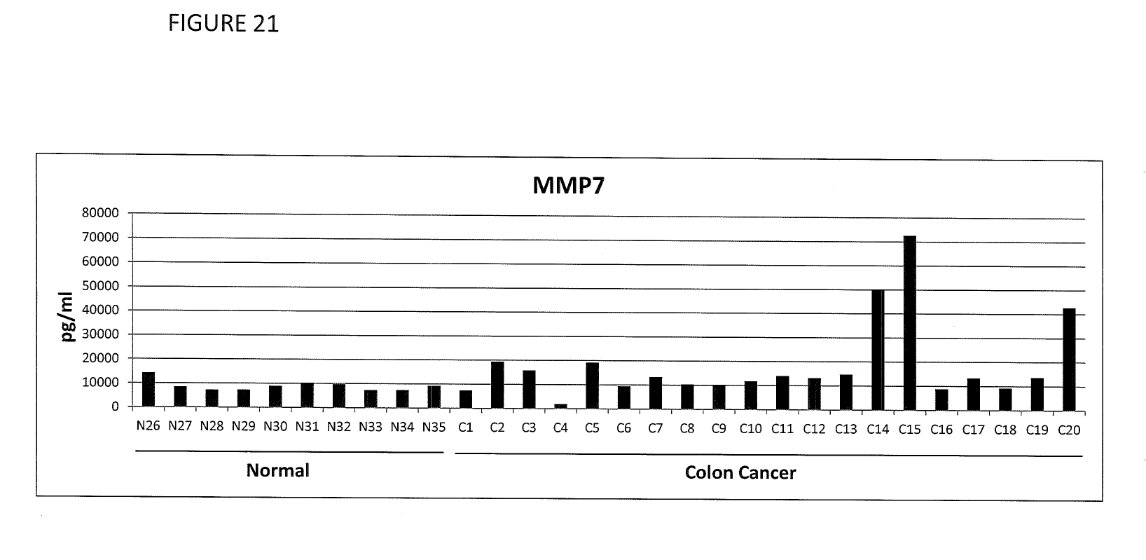

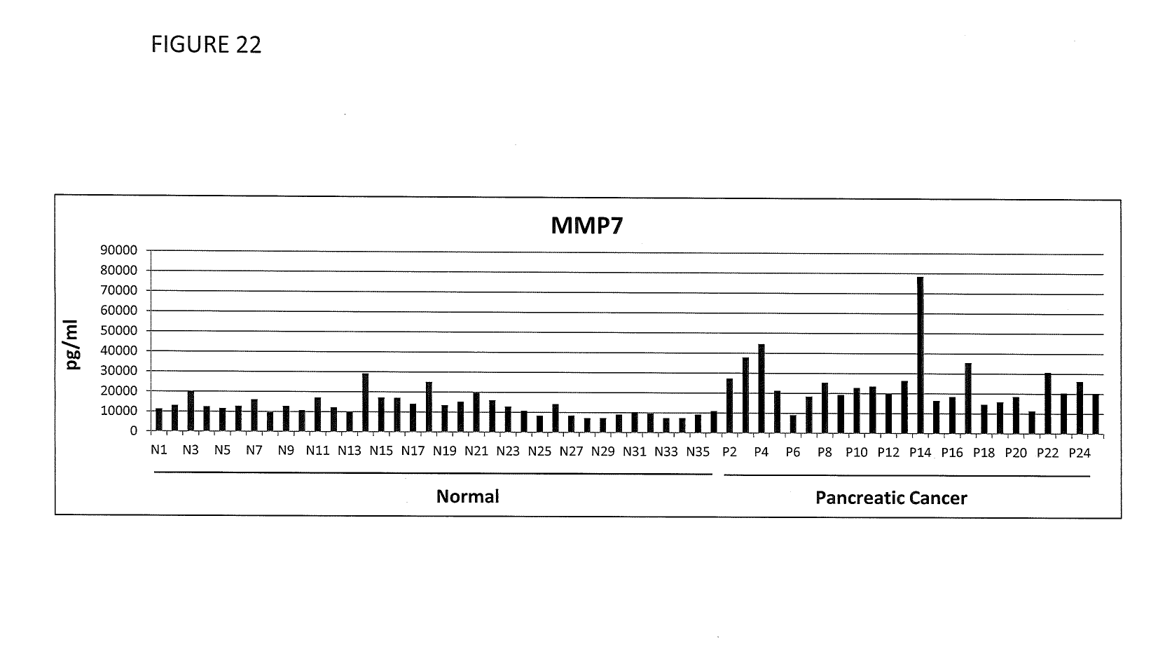

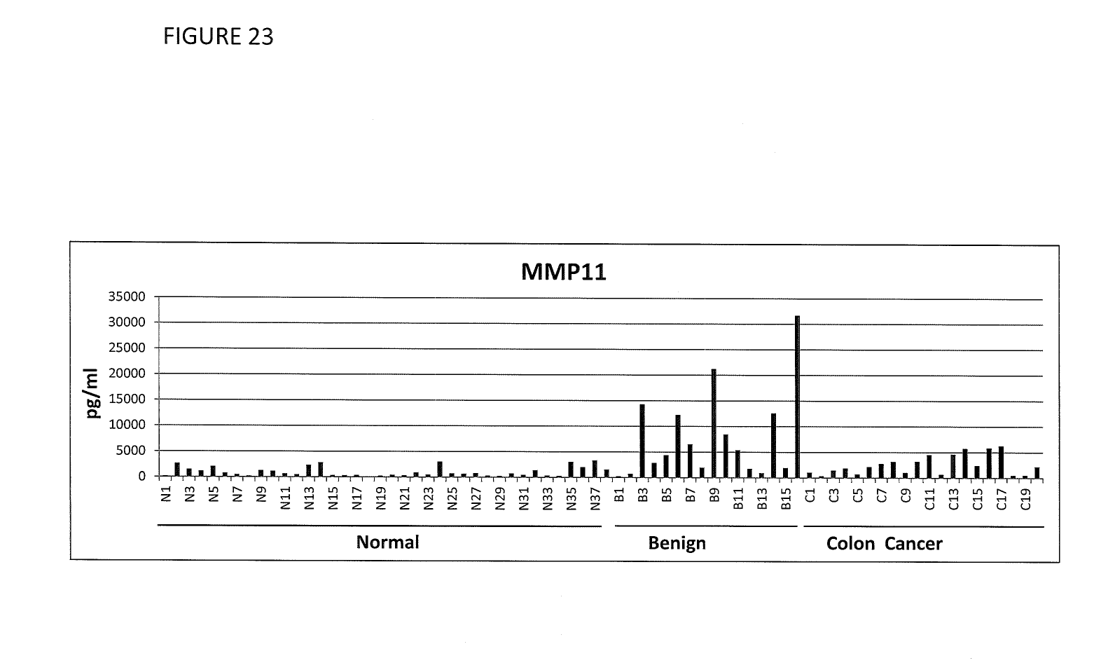

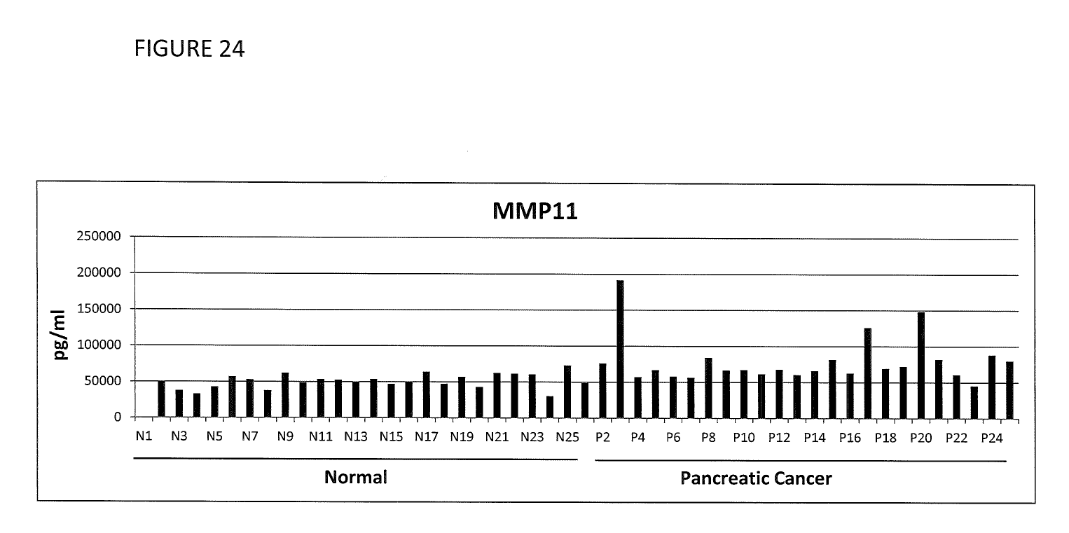

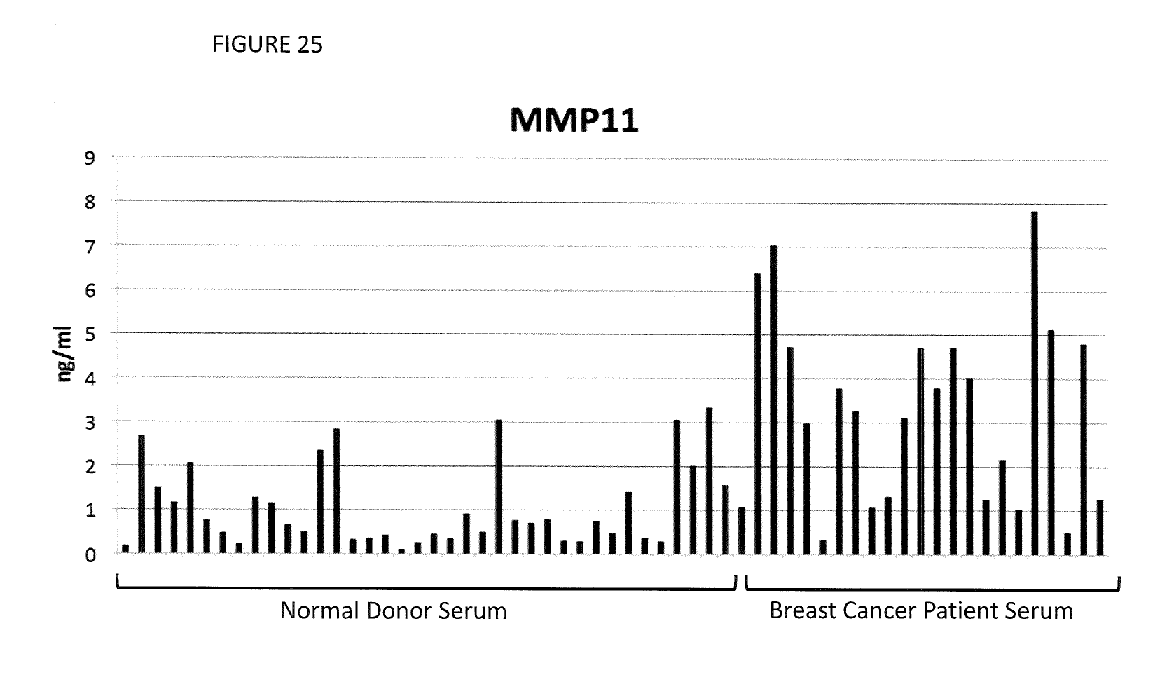

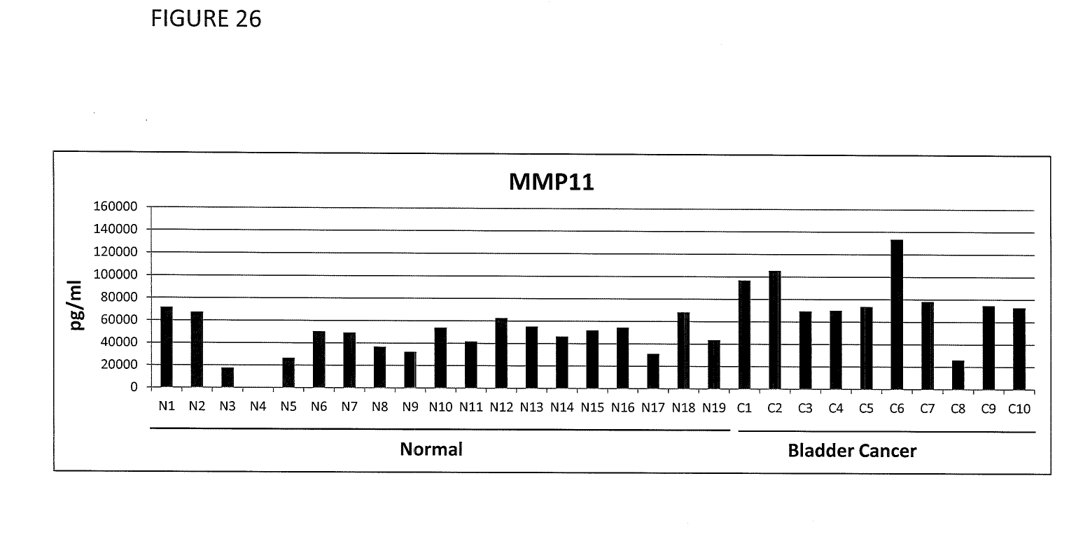

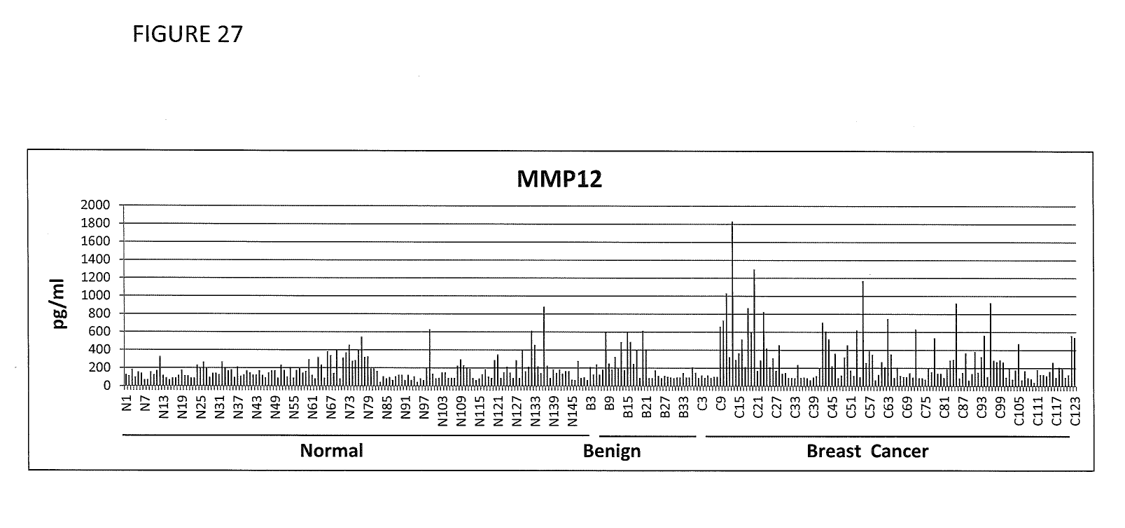

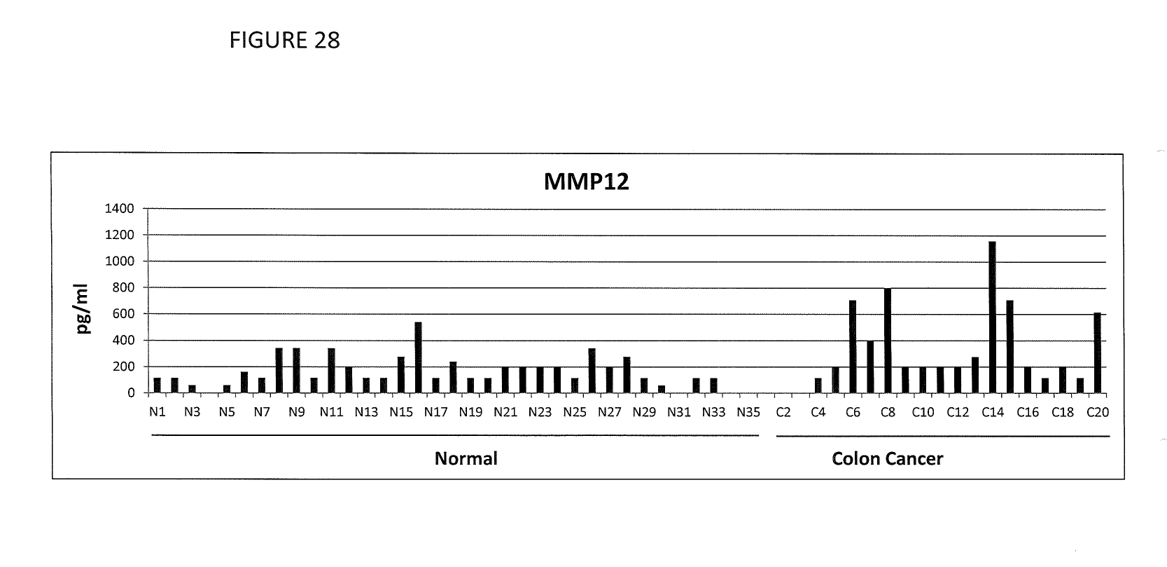

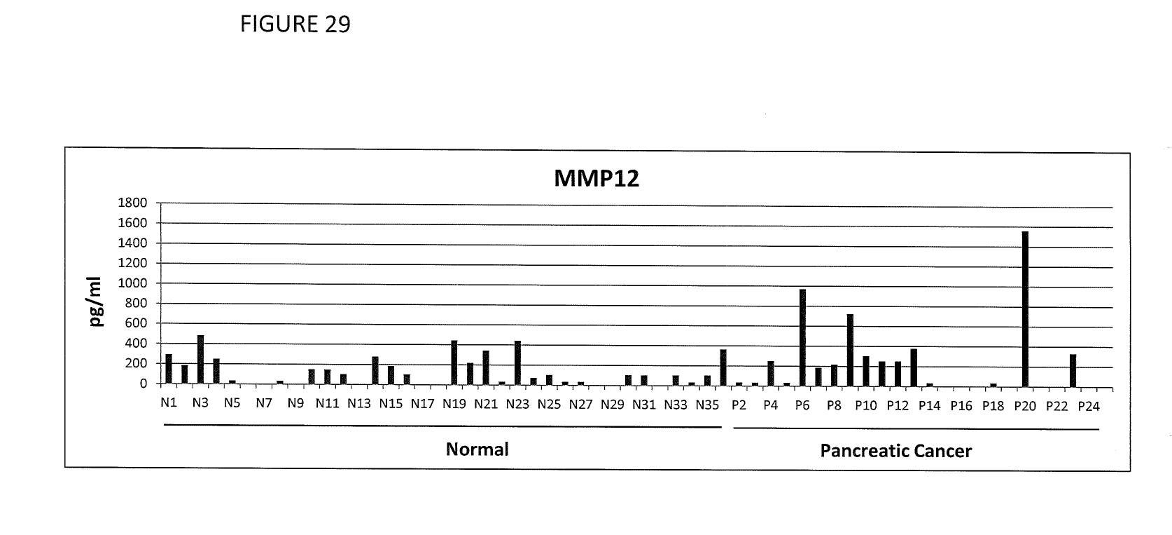

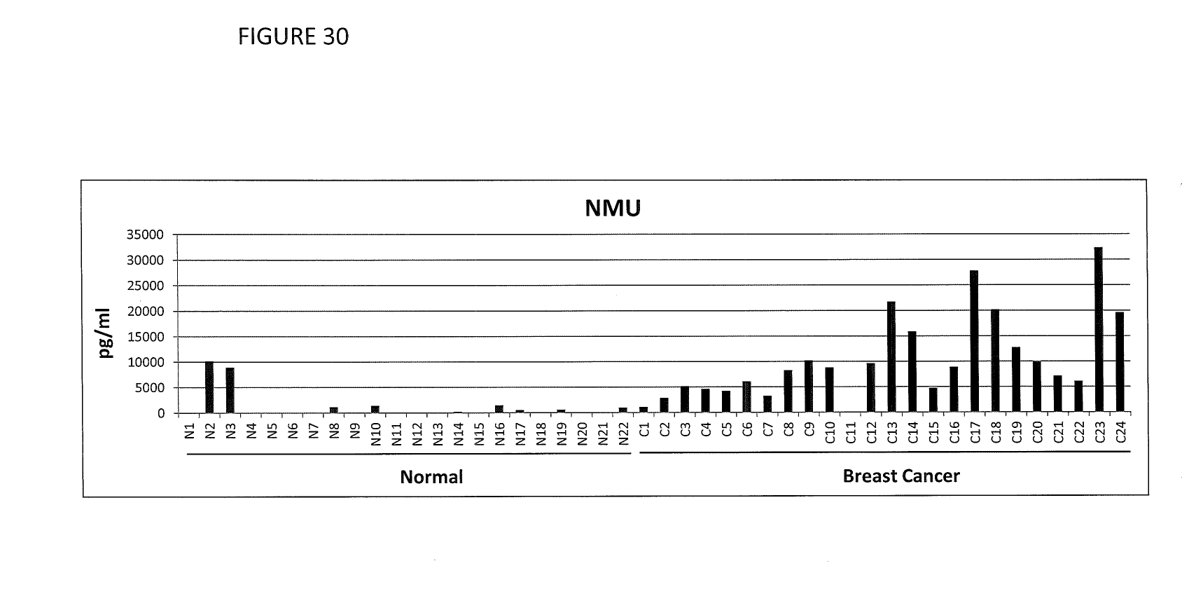

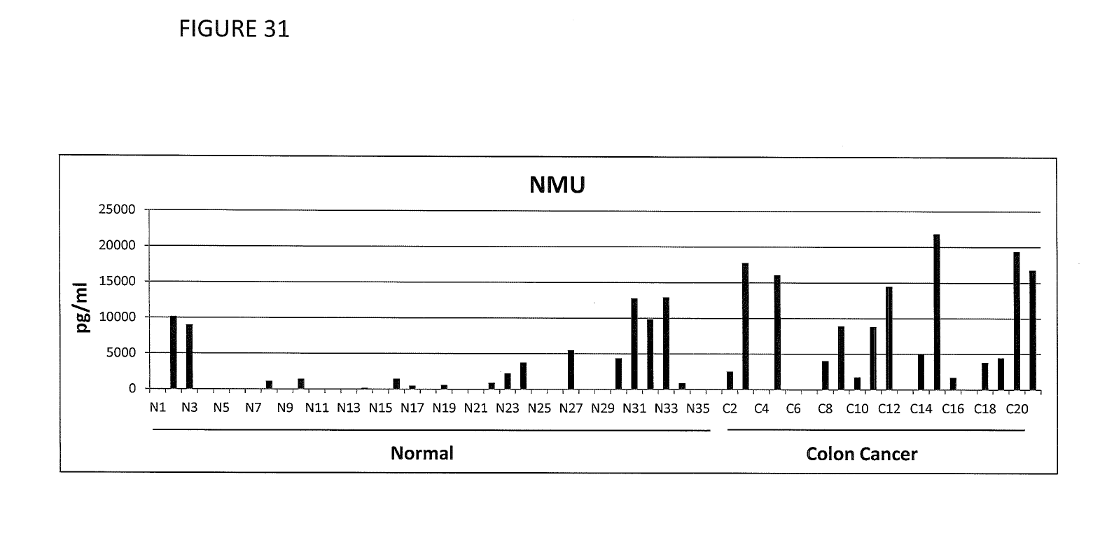

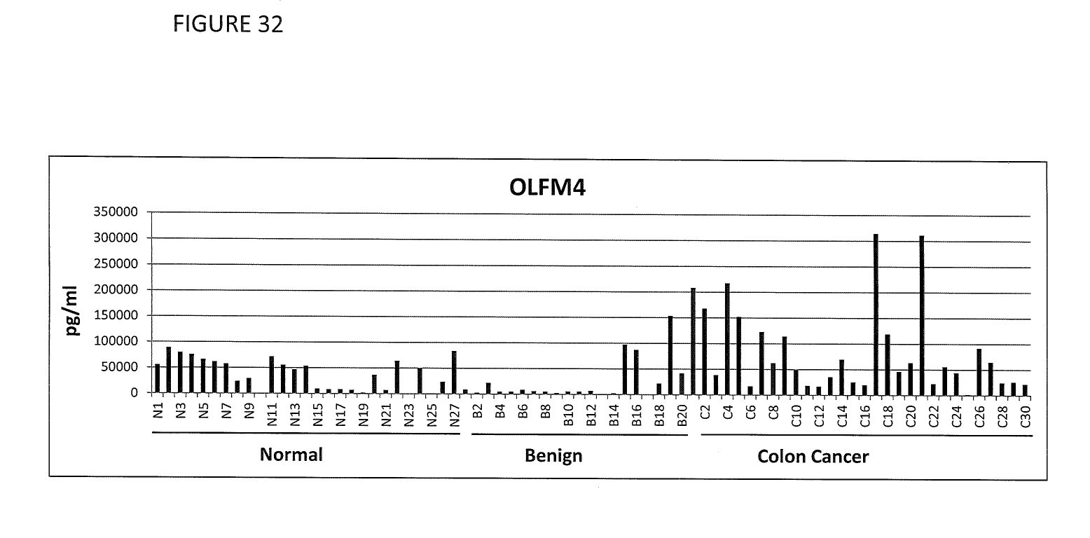

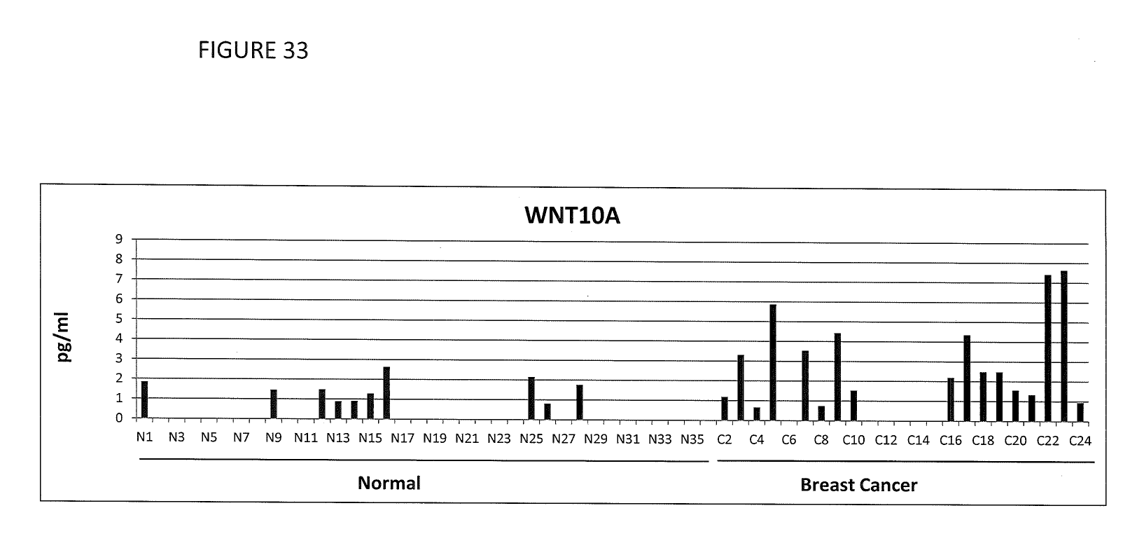

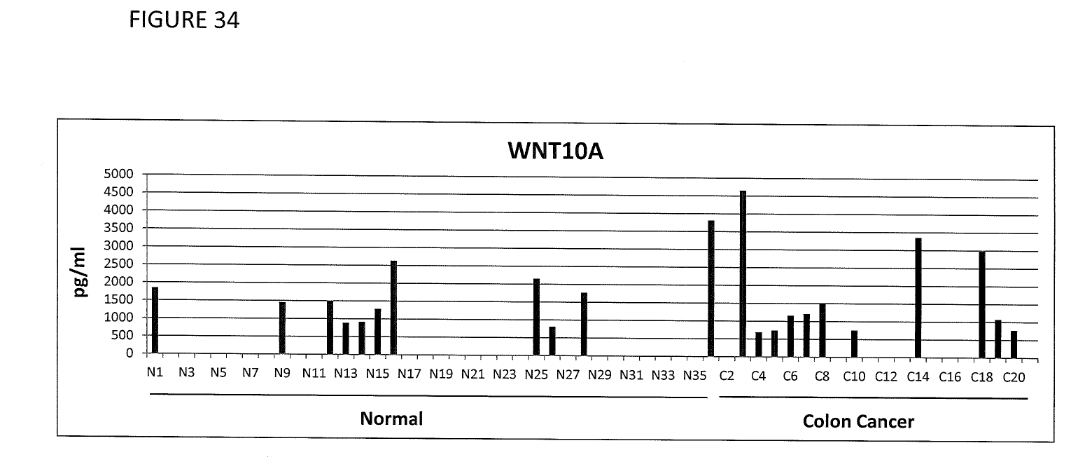

The results shown in FIGS. 10-34 indicated that each of the markers analyzed were found to be elevated in the serum of various cancer patients compared to normal samples obtained from cancer free subjects.

### Example 11

qPCR was used to investigate the expression level of the following genes in various cancers, benign tumors and normal tissues: AMH_{—}1038; ASCL1_{—}1095; C12orf56; C2orf70_{—}1010; COL10A, DSCR6_{—}1066; DSCR8_{—}1036; LHX8_{—}1283; MMP11; MMP12; NMU; SLC35D.

PCR primers were designed to be specific for the gene transcript of interest using the Standard Nucleotide BLAST program (NCBI) and to span at least one exon junction. Primers were chosen to have Tms of 58-63° C. calculated with the Breslauer equation, deltaG values >25Kcal/mol and displaying no self-complementarity using Oligo Calc software. Primers were ordered salt-free purified from the manufacturer (Eurofins MWG)

RNA was derived from commercial sources (Asterand; OriGene) and cDNA prepared using the SuperScript III First-Strand Synthesis System for RT-PCR (Invitrogen Cat. No. 18080-051) following the random hexamer protocol. Initial validation of primers assessed three major criteria: robustness, linearity and specificity. Acceptance criteria for absolute value robustness was that the final 2̂delta Ct value after subtracting housekeeping genes (GAPDH and GUSB) Ct values >1. Robustness in terms of differentiating disease from benign or normal samples required >2Ct difference of known positive over negative samples, as determined previously by microarray analysis (Illumina). To assess linearity, primers were used to amplify ten-fold dilutions of cDNA. Only primers exhibiting at or near the expected 3.3 Ct shift upon ten-fold dilution of template proceeded for further testing. Specificity was determined both by gel electrophoresis and from observing a single Tm generated from melting curve analysis on the instrument. PCR products were run on a 2% agarose gel and only those generating a single band of expected size passed validation.

Protocols of initial primer validation differed from external validation performed on OriGene TissueScan qPCR arrays chiefly in terms of volume and cDNA target.

PCR Protocol for Initial Primer Validation:

| Reagent | 1 Rx (μL) | Final Conc |
|---|---|---|
| 2X Power SYBR Green Master Mix | 10.0 | 1X |
| (Invitrogen Cat #4368706) | | |
| 100 μM F Primer (Eurofins MWG) | 0.20 | 1 μM |
| 100 μM R Primer (Eurofins MWG) | 0.20 | 1 μM |
| 10 or 1 ng/μL cDNA Template | 1.00 | |
| Molecular Biology grade H₂O | 18.6 | |
| (Cellgro Cat No 46-000-CM) | | |
| | 20.0 | |

| | Thermoprogram used on | |
|---|---|---|
| PCR Instruments | both Instruments: | |
| ABI 7500 Real Time PCR System | Activation | 50° C. 2:00 |
| ABI 7900HT Sequence Detection System | Denature | 95° C. 10:00 |
| | 40 Cycles | 95° C. 0:15 |
| | | 60° C. 1:00 |
| | Dissociation | 95° C. 0:15 |
| | | 60° C. 0:15 |
| | | 95° C. 0:15 |

The primers used are provided below in Table 4 (forward primers) and Table 5 (reverse primers):

TABLE 4

| Gene | | | |
|---|---|---|---|
| Marker | Forward Primer | Forward Primer Sequence | Accession # |
| AMH | JK1038-AMH-F | CGCCTGGAGGAGCTGGC (SEQ ID NO: 29) | NM_000479.3 |
| ASCL1 | JK1095-ASCL1-F | AATGGACTTTGGAAGCAGGGTGATC | NM_004316.2 |
| C12orf56 | JK1052-C12orf56-F | ACTCTAGCTGAGTATATTAGGAATAAC | NM_001099676.1 |
| C2orf70 | JK1010-C2orf70-F | CCACCGTCCTGCCTCCTC | NM_001105519.1 |
| COL10A1 | ES577-COL10A1-F | GGGCCTCAATGGACCCACCG | NM_000493.3 |
| COL10A1 | JK1341-COL10A1-F | CAATGGACCCACCGGGCCAC | NM_000493.3 |
| DSCR6 | JK1066-DSCR6-F | ATCCAGACACCTGGAGATGCTG | NM_018962.2 |
| DSCR8 | JK1036-DSCR8-F | ATGCCTAATCCCAGCTTCATC | NR_026838.1 |
| LHX8 | JK1283-LHX8-F | CTCGGACCAGCTTTACAGCAGATC | NM_001001933.1 |
| MMP11 | JK1178-MMP11-F | ACCGCTGGAGCCAGACGCC | NM_005940.3 |
| MMP12 | JK1192-MMP12-F | TCTGGACTACACATTCAGGAGGCAC | NM_002426.2 |
| NMU | JK1210-NMU-F | TCTTTTCTGTCCATTGATTCTCAGCCTC | NM_006681.2 |
| SLC35D3 | JK1024-SLC35D3-F | GCTATTTTGAAAATATGAGTTCTTAGC | NM_001008783.1 |

TABLE 5

| Gene | | | |
|---|---|---|---|
| Marker | Reverse Primer | Reverse Primer Sequence | Accession # |
| AMH | JK1009-AMH-R | CCGGGAGTCCTCTCCGC | NM_000479.3 |
| ASCL1 | JK1096-ASCL1-R | TAGTTGGCGATGGGGTTGGTTGAC | NM_004316.2 |
| | JK1053-C12orf56- | | |
| C12orf56 | R | ATGGGGTAACACAATGGGAGC | NM_001099676.1 |
| C2orf70 | JK1011-C2orf70-R | CATCAGGCTCTGCTCTGAAC | NM_001105519.1 |
| COL10A1 | ES578-COL10A1-R | CTGGGCCTTTGGCCTGCCTT | NM_000493.3 |
| | JK1342-COL10A1- | | |
| COL10A1 | R | AGACTGGGCCTTTGGCCTGC | NM_000493.3 |
| DSCR6 | JK1067-DSCR6-R | ACTCCGCAGGTATTCTTGACGC | NM_018962.2 |
| DSCR8 | JK1037-DSCR8-R | GAAAATGTATGAGCCAGCCTTC | NR_026838.1 |
| LHX8 | JK1284-LHX8-R | ACGTGTTTCTTGTGGCGTGCTCTAC | NM_001001933.1 |
| MMP11 | JK1179-MMP11-R | CGAGAGGCCAATGCTGGGTAGC | NM_005940.3 |
| MMP12 | JK1193-MMP12-R | GTCACAGAGAGCTGGTTCTGAATTGTC | NM_002426.2 |
| NMU | JK1211-NMU-R | CTCTCATGCAGGTGAGGAACGAGC | NM_006681.2 |
| SLC35D3 | JK1025-SLC35D3-R | CTTTACAGGTGGTCCCTCTTC | NM_001008783.1 |

PCR Protocol for OriGene TissueScan Arrays:

| Reagent | 1 Rx (μL) | Final Conc |
|---|---|---|
| 2X Power SYBR Green Master Mix | 15.0 | 1X |
| (Invitrogen Cat #4368706) | | |
| 100 μM F Primer (Eurofins MWG) | 0.30 | 1 μM |
| 100 μM R Primer (Eurofins MWG) | 0.30 | 1 μM |
| Molecular Biology grade H₂O | 14.4 | |
| (Cellgro Cat No 46-000-CM) | | |
| | 30.0 | |

| PCR Instruments | Thermoprogram used: | |
|---|---|---|
| ABI 7500 Real Time PCR System | Activation | 50° C. 2:00 |
| | Denature | 95° C. 10:00 |
| | 42 Cycles | 95° C. 0:15 |
| | | 60° C. 1:00 |
| | | (72° C. 0:10) |
| | | Used with |
| | | amplicons >120 bp |
| | Dissociation | 95° C. 0:15 |
| | | 60° C. 0:15 |
| | | 95° C. 0:15 |

Initial validation experiments were performed using RNA derived from commercial sources (Asterand, Detroit, Mich.; OriGene, Rockville, Md.) and prepared into cDNA using the SuperScript III First-Strand Synthesis System for RT-PCR (Life Technologies, Carlsbad, Calif.) following the random hexamer protocol. The samples were amplified in quantitative reverse-transcriptase PCR (qRT-PCR) reactions with 1 uM final concentration of each of the forward and reverse primers (Eurofins MWG Huntsville, Ala.) using the Power SYBR Green Master Mix Kit (Life Technologies, Carlsbad, Calif.) following the manufacturer's instructions. Sample input was between 3 to 10 ng of cDNA in a final reaction volume of 20 uL. The real-time PCR instruments used were the ABI 7500 Real Time PCR System or the ABI 7900HT Sequence Detection System with the thermoprogram set for 50° C. for 2 minutes, then 95° C. for 10 minutes, followed by 40 cycles of 95° C. for 15 seconds and 60° C. for 1 minute. Dissociation analysis was immediately performed using 95° C. for 15 seconds, 60° C. for 15 seconds and 95° C. for 15 seconds.

Primers demonstrating good correlation and specificity for cancer, as well as exhibiting robustness and linear dose response to sample input proceeded for further testing. TissueScan qPCR arrays (OriGene, Rockville, Md.) were used to test larger number of cDNA samples. The lyophilized cDNA in each well of the array was mixed with 1 uM final concentration of each of the forward and reverse primers using the Power SYBR Green Master Mix Kit (Life Technologies, Carlsbad, Calif.) in a final reaction volume of 30 uL. The real-time PCR instrument used was the ABI 7500 Real Time PCR System with the thermoprogram set for 50° C. for 2 minutes, then 95° C. for 10 minutes, followed by 40 cycles of 95° C. for 15 seconds and 60° C. for 1 minute. Dissociation analysis was immediately performed using 95° C. for 15 seconds, 60° C. for 15 seconds and 95° C. for 15 seconds.

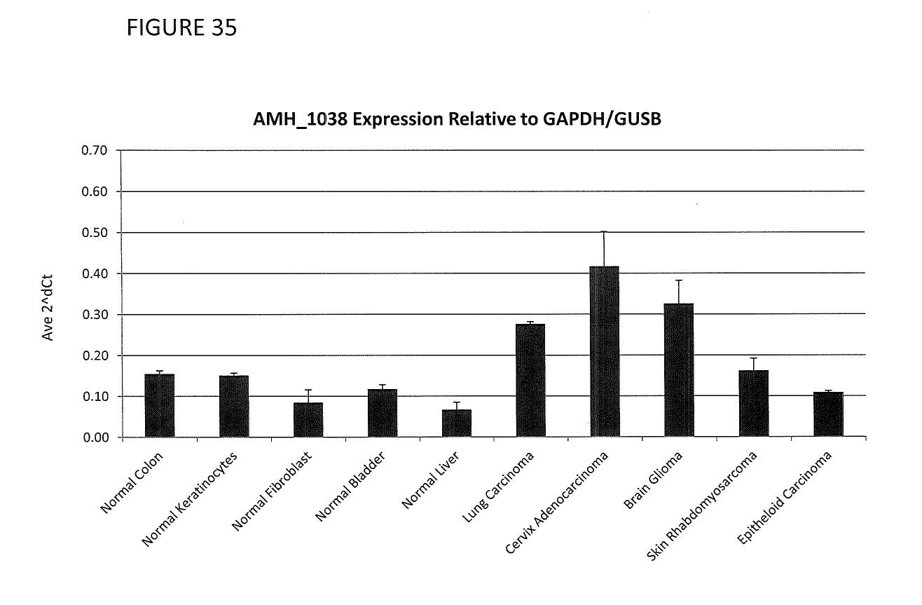

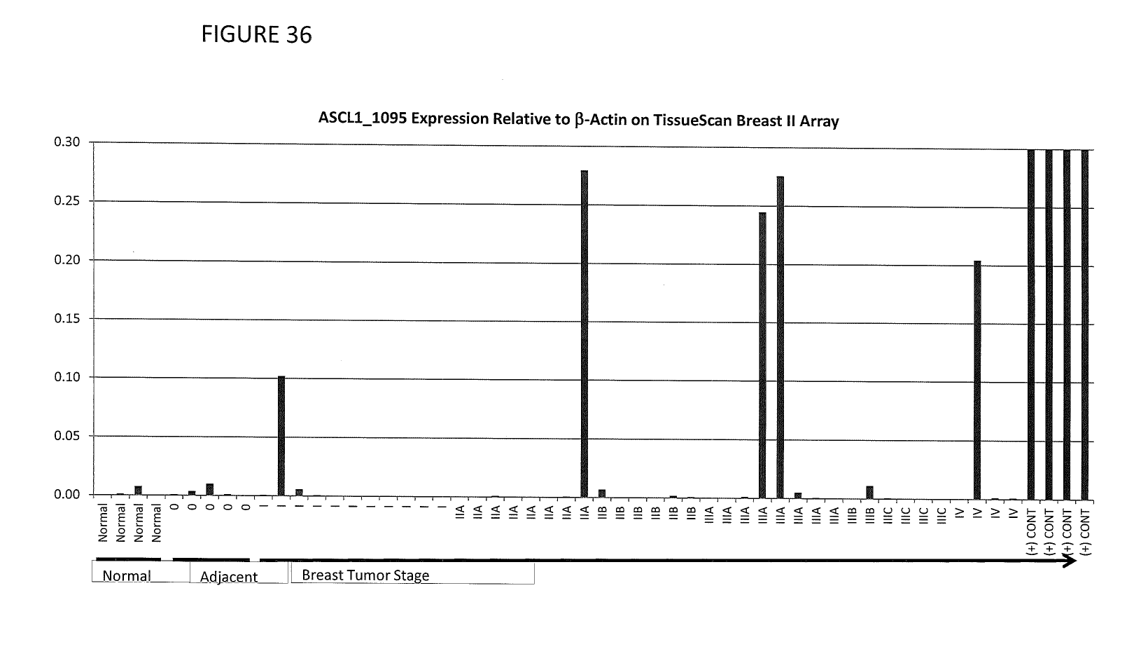

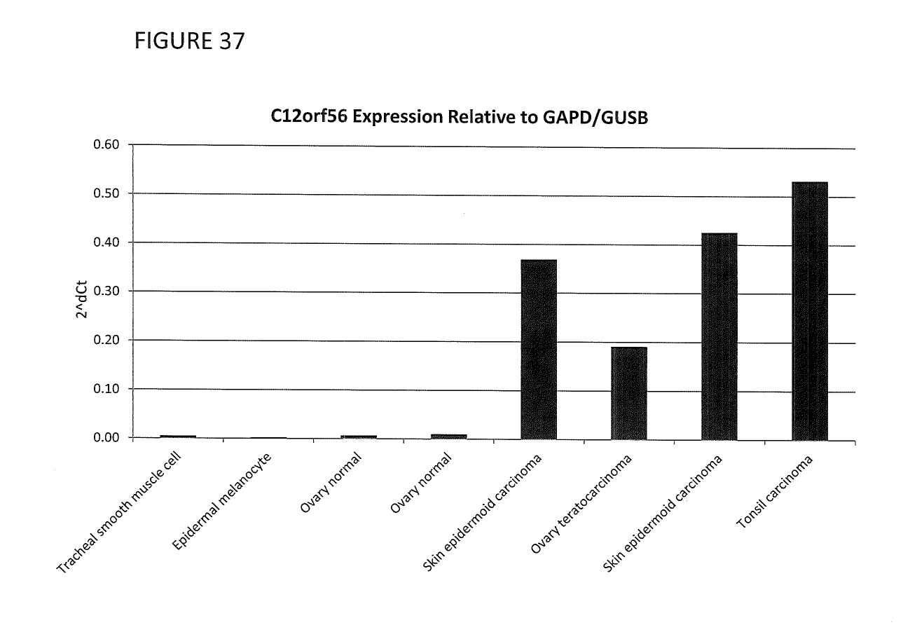

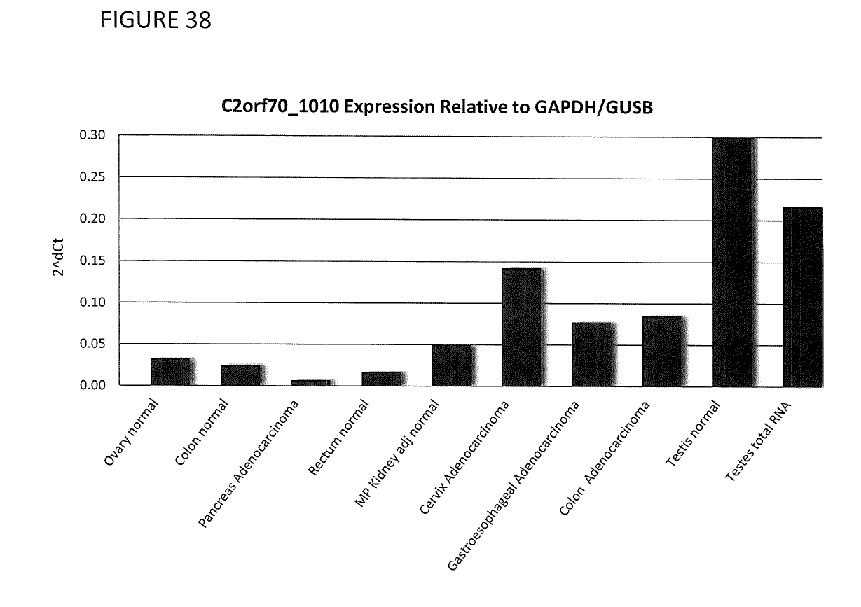

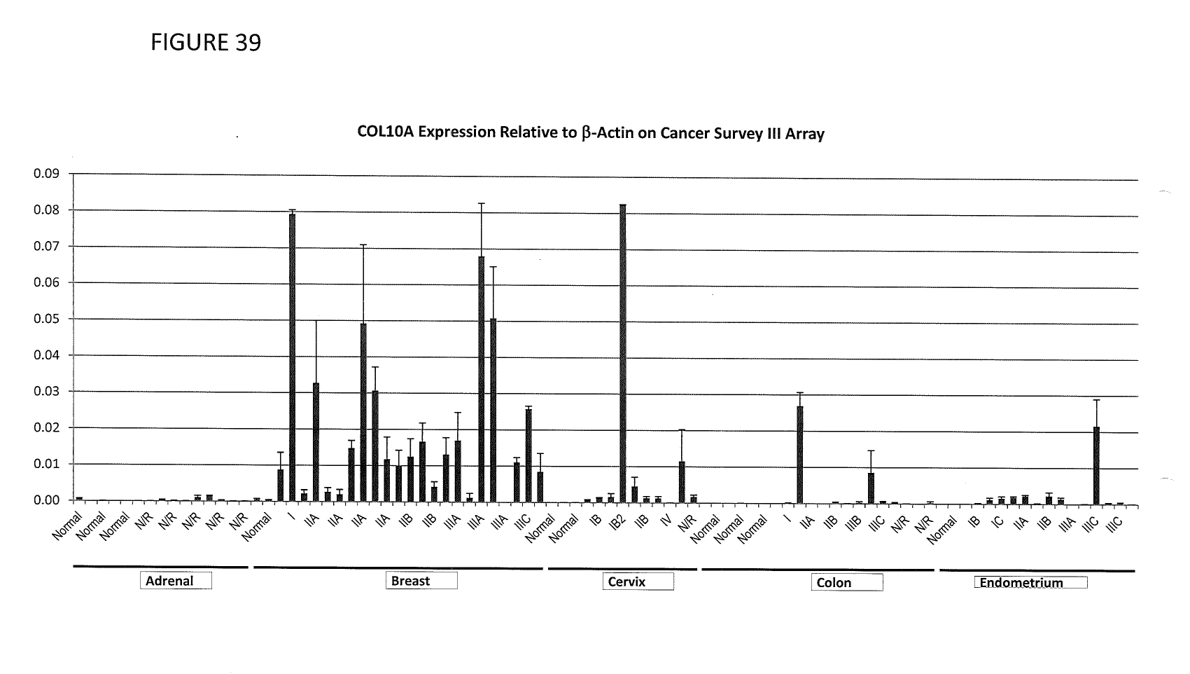

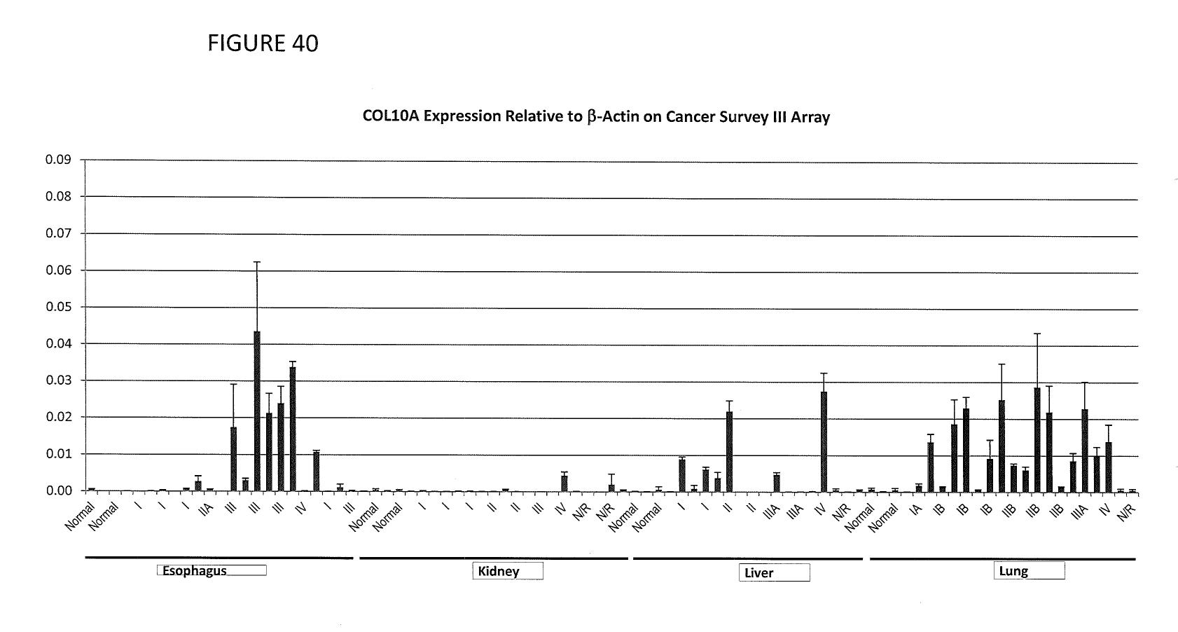

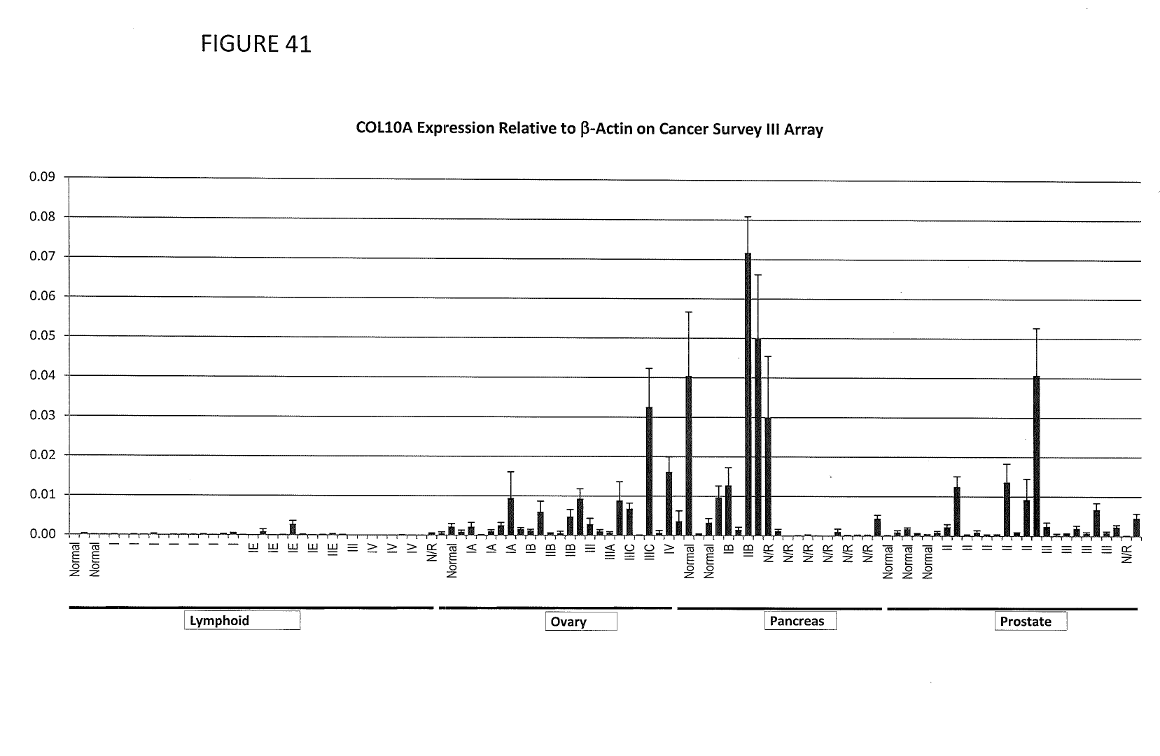

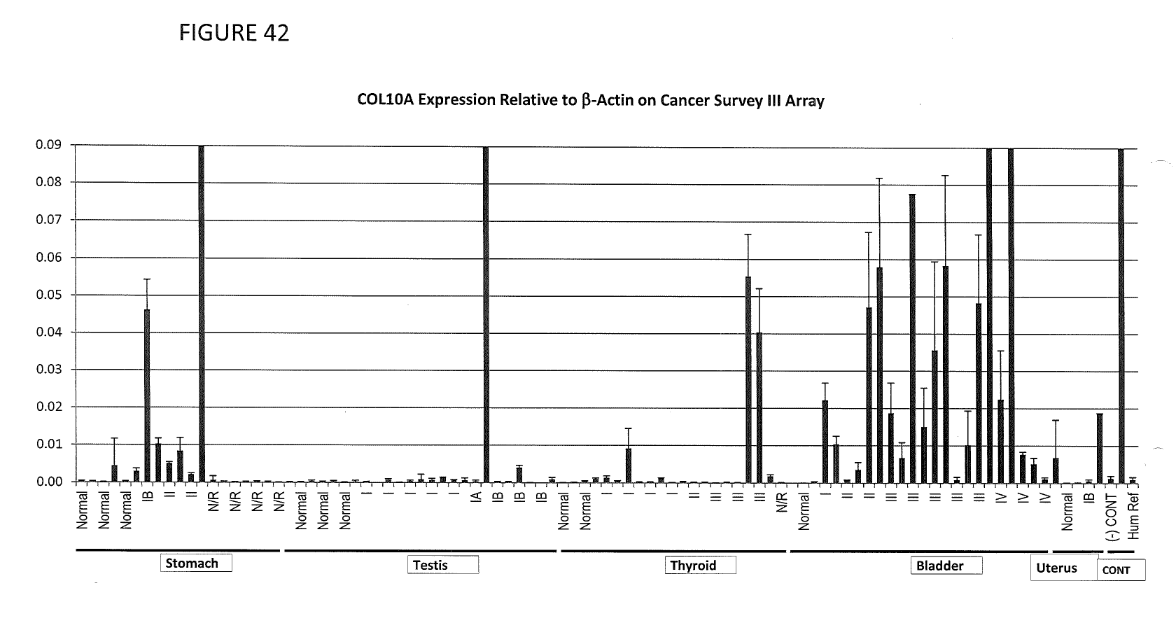

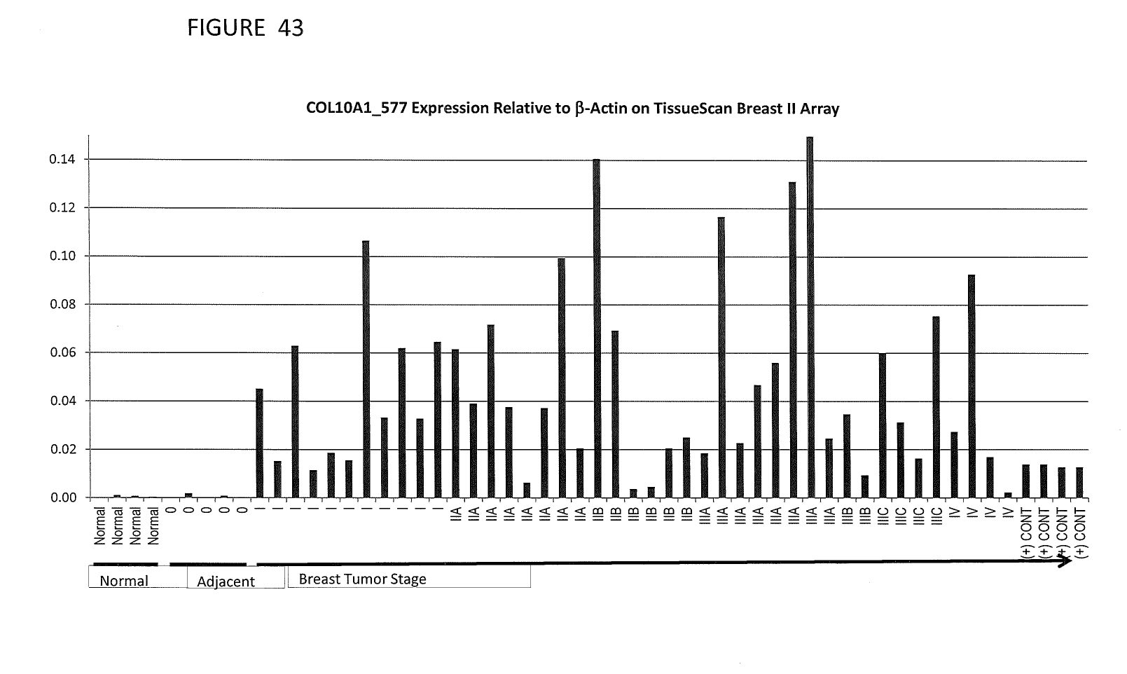

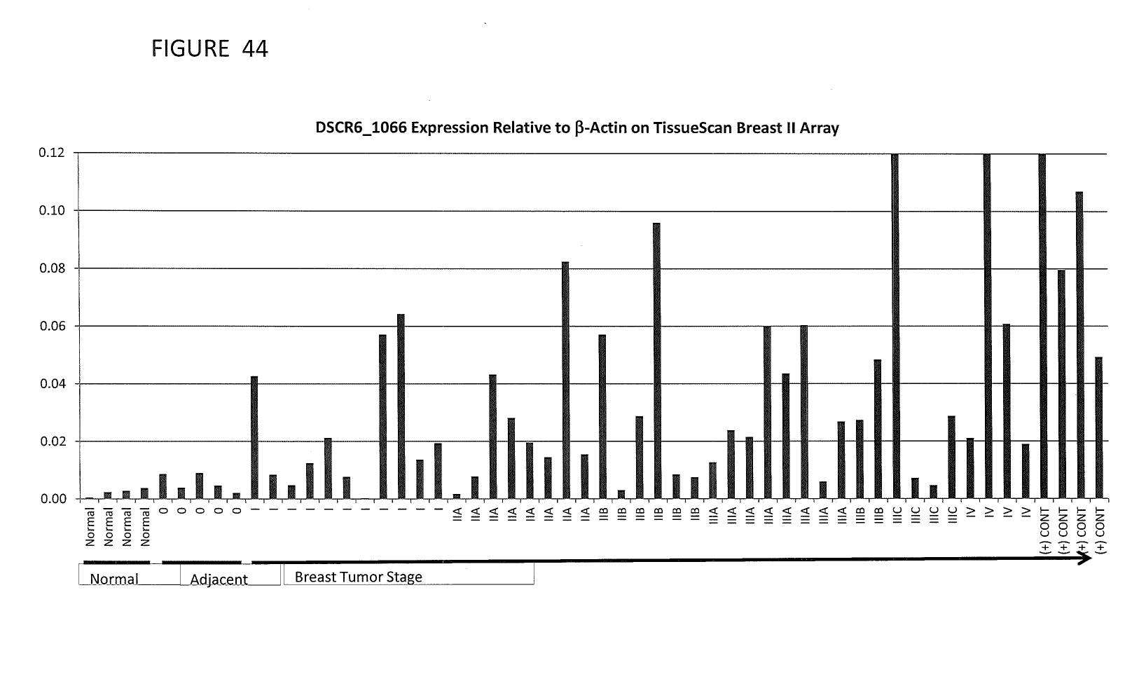

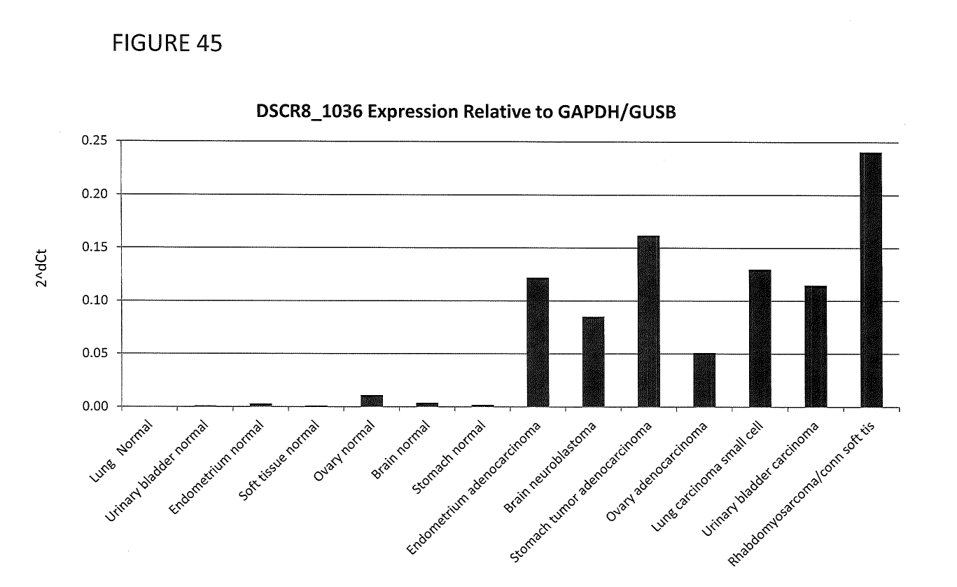

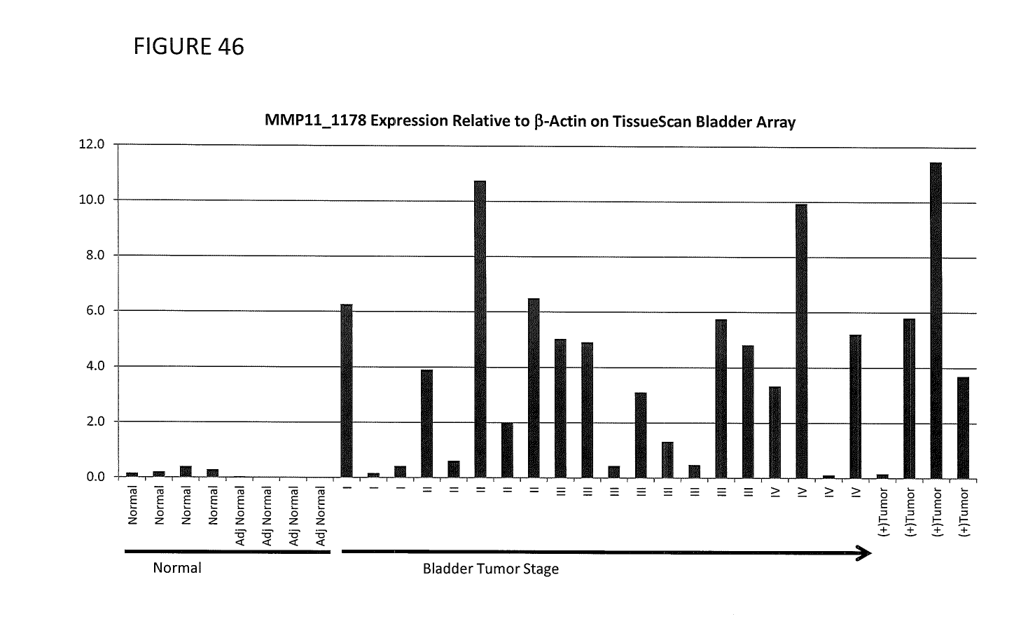

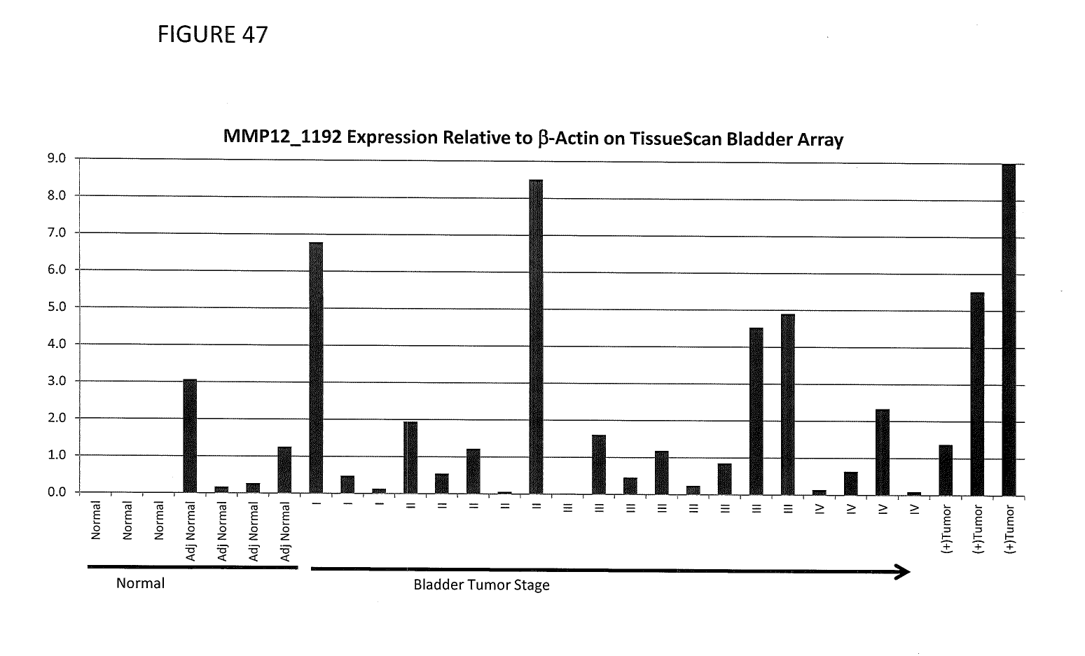

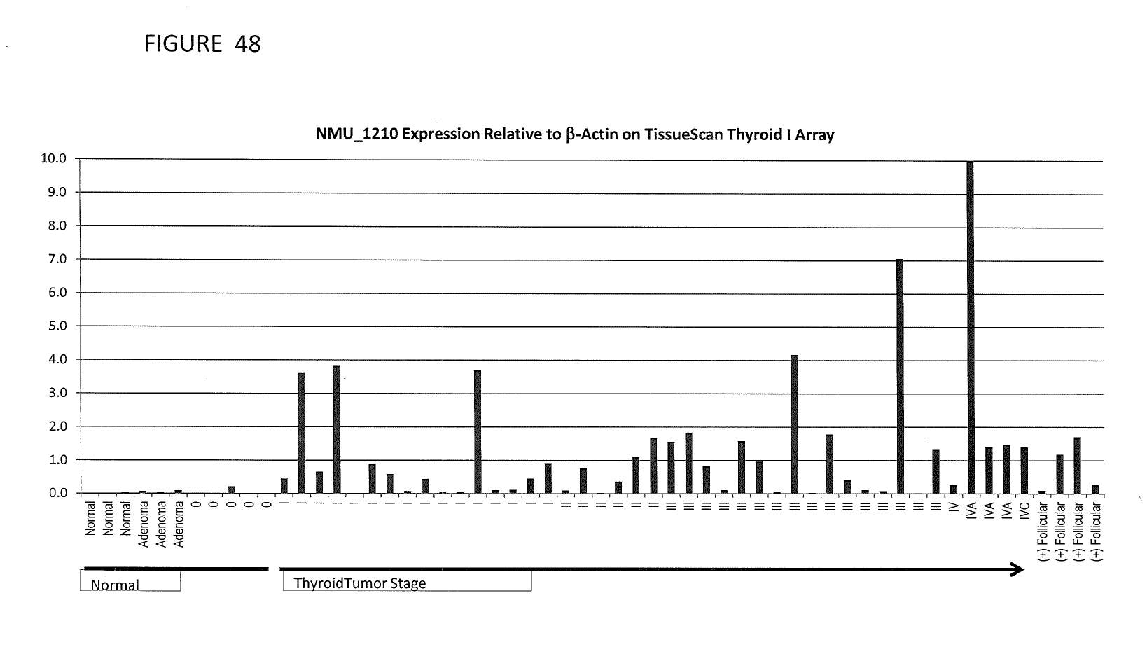

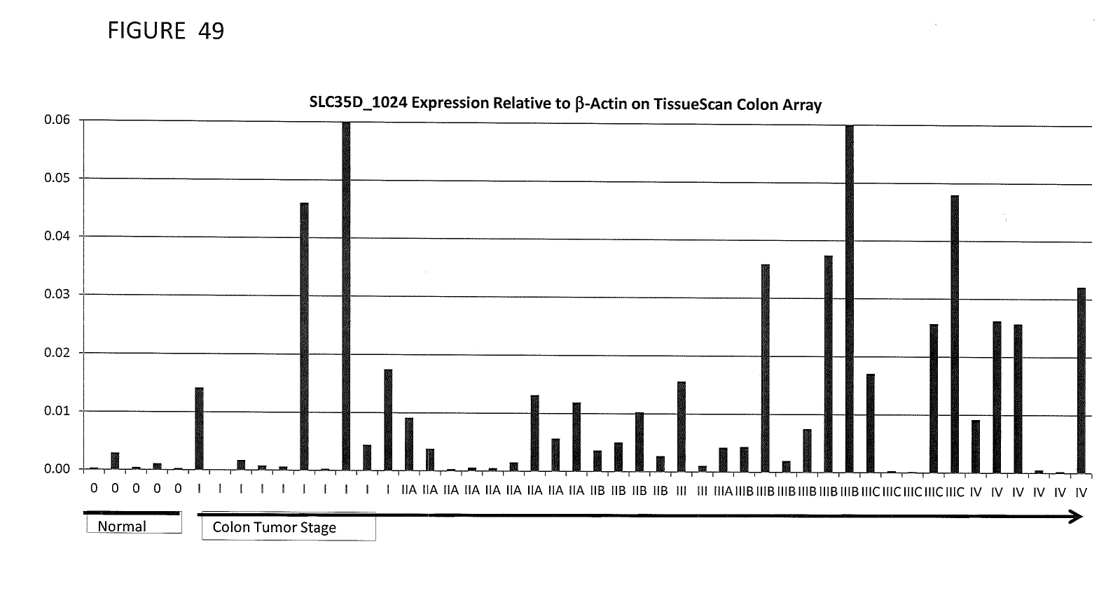

The results are presented in FIGS. 35-50 and show that the markers AMH_{—}1038; ASCL1_{—}1095; C12orf56; C2orf70_{—}1010; COL10A, DSCR6_{—}1066; DSCR8_{—}1036; LHX8_{—}1283; MMP11; MMP12; NMU; SLC35D are elevated in various cancer types.

### Example 12

Expression of POTE was investigated by Immunofluoresence using tissue obtained from a breast cancer ductal carcinoma, a fibroadenoma and normal breast tissue.

Paraffin embedded tissue sections were obtained from Asterand (Detroit, Mich.). These specimens included: Normal breast tissue (donors with no history of cancer), fibroadenoma of the breast, breast ductal cell carcinoma, normal thyroid tissue (donors with no history of cancer), thyroid follicular adenoma and thyroid follicular carcinoma. Prior to the staining with antibodies, the sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95° C. 40 minutes using an IHC-Steamer Set (IHC World #IW-1102). Immunostaining was performed using a monoclonal mouse anti-human POTE antibody (Kindly donated from Dr. Ira Pastan) at a 1:100 dilution. The primary antibody was detected using an Alexa Fluor 594 Goat anti-mouse IgG (Life Sciences #A11032) at a 1:200 dilution.

Vectashield mounting medium with DAPI was used to preserve the stained samples (Vector Laboratories #H-1200). Images were taken with an exposure time of 400 milliseconds using a Nikon Eclipse TE2000-U at a magnification of 10,000 and an X-Cite 120 fluorescence illumination system (Lumen Dynamics).

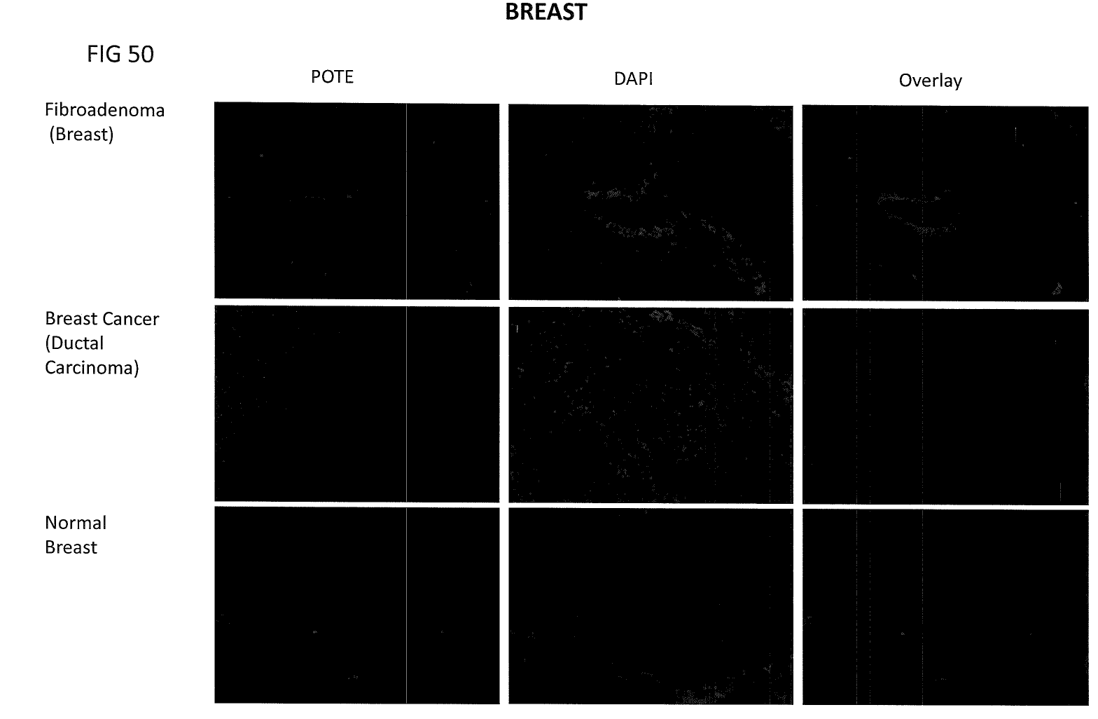

The results are shown in FIG. 50 and demonstrate that POTE is expressed in breast cancer tissue.

### Example 13

Expression of MMP11 was investigated by Immunofluoresence using tissue obtained from a breast cancer ductal carcinoma, a fibroadenoma and normal breast tissue.

Paraffin embedded tissue sections were obtained from Asterand (Detroit, Mich.). These specimens included: Normal breast tissue (donors with no history of cancer), fibroadenoma of the breast, and breast ductal cell carcinoma. Prior to the staining with antibodies, the sections were dewaxed in xylene and rehydrated in cycles of ethanol (100%, 95%, 70%) followed by a wash in distilled water. Antigen retrieval was performed in epitope retrieval buffer (IHC World #IW-1100) by incubating the slides at 95° C. 40 minutes using an IHC-Steamer Set (IHC World #IW-1102). Immunostaining was performed using a polyclonal rabbit anti-human MMP11 antibody (Abeam #ab52904) at a 1:100 dilution. The primary antibody was detected using an Alexa Fluor 594 Donkey anti-rabbit IgG (Life Sciences #A21207) at a 1:200 dilution.

Vectashield mounting medium with DAPI was used to preserve the stained samples (Vector Laboratories #H-1200). Images were taken with an exposure time of 400 milliseconds using a Nikon Eclipse TE2000-U at a magnification of 10,000 and an X-Cite 120 fluorescence illumination system (Lumen Dynamics).

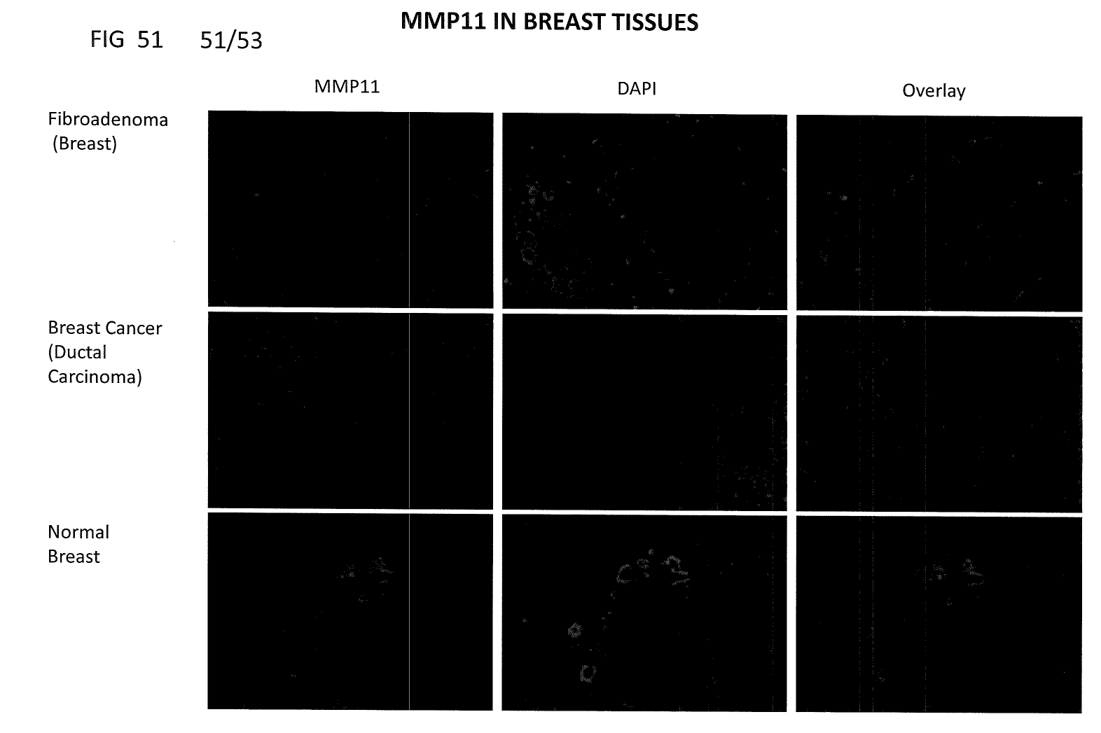

The results are shown in FIG. 51 and demonstrate that MMP11 is expressed in breast cancer tissue.

### Example 14

Quantitative reverse transcription-polymerase chain reaction (qRT-PCR) was used to investigate expression of the genes L1TD1 and APOBEC1 in colon cancer tissue and normal colon tissue.

Total RNA was extracted with the RNeasy Mini Kit (Qiagen) and cDNA generated using the SuperScript III reverse transcriptase in combination with random hexamer primers alone or in combination with oligo-dT primers (all reverse transcription components from Invitrogen/Life Technologies). PCRs were carried out on a 7900HT Sequence Detection System or a 7500 Real Time PCR System (Applied Biosystems/Life Technologies) utilizing SYBR® Green I (Applied Biosystems/Life Technologies) or TaqMan chemistries. TaqMan PCR was conducted with probes from the Universal Probe Library (UPL) (Roche) in combination with correspondingly designed primers. Background: The UPL System contains a relatively small number of short hydrolysis probes that cover an extensive proportion of the human mRNA transcriptome. UPL probes contain locked nucleic acids (LNAs) which increase the probes' melting temperatures. This allows the probe and the longer, unmodified, primers to anneal at the same temperature.

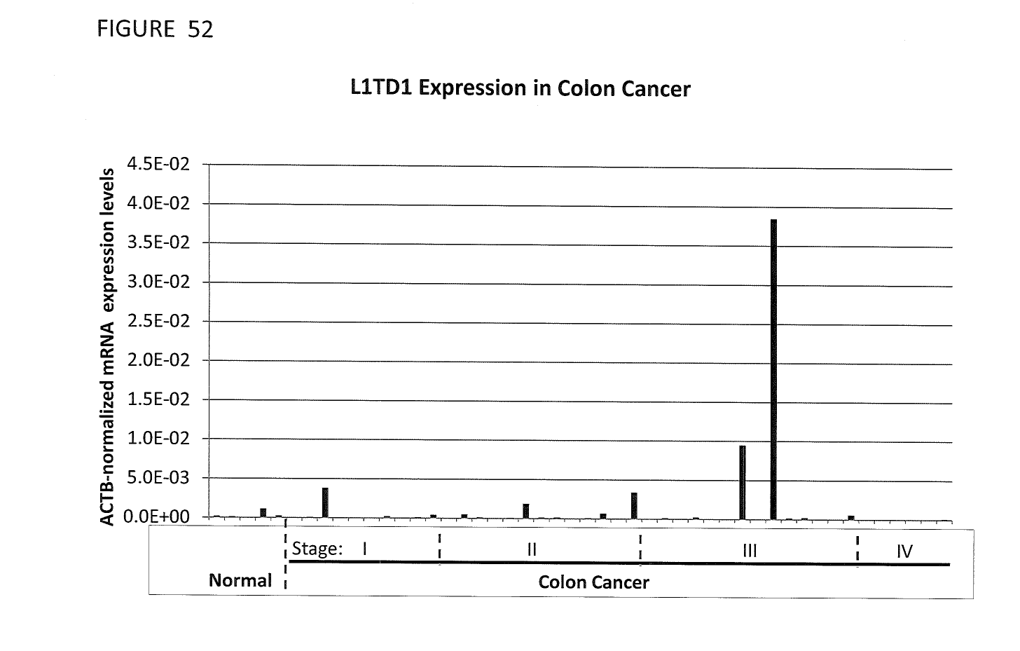

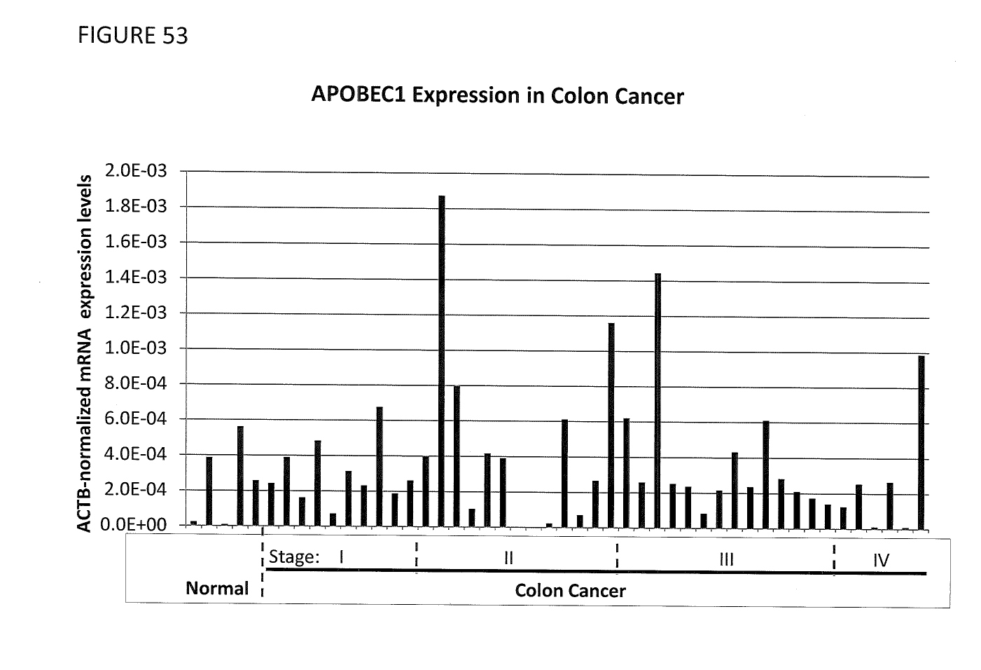

The results are shown in FIGS. 52-53 and demonstrate that L1TD1 and APOBEC 1 are both expressed at elevated levels in colon cancer tissue relative to normal colon tissue.

TABLE 1

| Symbol | Accession | Definition | Probe_Id | Probe Sequence |
|---|---|---|---|---|
| PRAME | NM_206955.1 | Homo sapiens preferentially expressed antigen in | ILMN_2306033 | GACCCACGTGCTGTATCCTGTCCCCCTGGAGAGTTATG |
| | | melanoma (PRAME), transcript variant 4, mRNA. | | AGGACATCCATG |
| AMH | NM_000479.2 | Homo sapiens anti-Mullerian hormone (AMH), mRNA. | ILMN_1660995 | CTCATCAGCCTGTCGGAGGAACGCATCAGCGCGCACCA |
| | | | | CGTGCCCAACAT |
| C12orf56 | NM_001099676.1 | Homo sapiens chromosome 12 open reading frame 56 | ILMN_1770616 | TGCCAGCCTTGCAGAAAAGGCTCCCATTGTGTTACCCC |
| | | (C12orf56), mRNA. | | ATCACTCAACCT |
| DSCR6 | NM_018962.1 | Homo sapiens Down syndrome critical region gene 6 | ILMN_1709257 | TAGGGAGTAGAACCGTCTCTCTTCTTAGTTGGTGACTG |
| | | (DSCR6), mRNA. | | TTTGGGGCCTGG |
| GNGT1 | NM_021955.3 | Homo sapiens guanine nucleotide binding protein | ILMN_2091100 | GTTGAAGAACGATCTGGCGAGGATCCACTGGTAAAGGG |
| | | (G protein), gamma transducing activity polypeptide 1 | | CATCCCAGAGGA |
| | | (GNGT1), mRNA. | | |
| SLC35D3 | NM_001008783.1 | Homo sapiens solute carrier family 35, member D3 | ILMN_1702419 | ACTGAAACCCAGCCAGAAGAGGGACCACCTGTAAAGCA |
| | | (SLC35D3), mRNA. | | AGTCCTTTCAAG |
| C2orf70 | NM_001105519.1 | Homo sapiens chromosome 2 open reading frame 70 | ILMN_3247753 | CCTGATGCCTGAGATCCTGGGTCGTGACCAGCCTGGCT |
| | | (C2orf70), mRNA. | | TGCTTGGAATAA |
| CELSR3 | NM_001407.2 | Homo sapiens cadherin, EGF LAG seven-pass G-type | ILMN_1691290 | CCCAGCGGCCCTCTTTCCTGTCTGTGTAAATTGTTCCG |
| | | receptor 3 (flamingo homolog, Drosophila) (CELSR3), | | TGAAGCCGCGCT |
| | | mRNA. | | |
| COL10A1 | NM_000493.3 | Homo sapiens collagen, type X, alpha 1 (COL10A1), mRNA. | ILMN_1672776 | CCCCTAAAATATTTCTGATGGTGCACTACTCTGAGGCC |
| | | | | TGTATGGCCCCT |
| DSCR8 | NM_203428.1 | Homo sapiens Down syndrome critical region gene 8 | ILMN_1763901 | TCCCACTTGGCAGGGGCCGTCTTGTCCACTCGTTTCTG |
| | | (DSCR8), transcript variant 2, mRNA. | | TAAACATGGGTG |
| LIN28B | NM_001004317.2 | Homo sapiens lin-28 homolog B (C. elegans) (LIN28B), | ILMN_1748697 | CTCGCATGCAGTCATCTGGAGGGACTGAAGCACTGTTT |
| | | mRNA. | | GCCTTTCTGTAC |
| MEST | NM_177524.1 | Homo sapiens mesoderm specific transcript homolog | ILMN_1669479 | GCGCAACCGGTTCTCCGAAACATGGAGTCCTGTAGGCA |
| | | (mouse) (MEST), transcript variant 2, mRNA. | | AGGTCTTACCTG |
| MMP12 | NM_002426.2 | Homo sapiens matrix metallopeptidase 12 (macrophage | ILMN_2073758 | TCTATTTGAAGCATGCTCTGTAAGTTGCTTCCTAACAT |
| | | elastase) (MMP12), mRNA. | | CCTTGGACTGAG |
| SBK1 | NM_001024401.2 | Homo sapiens SH3-binding domain kinase 1 (SBK1), mRNA. | ILMN_1728298 | CAGAGCCCCAGCCCCTCATGTCTTGCCGCCCTTCCTCC |
| | | | | ATGTGTTTGTAA |
| NIH_MGC_141 | BU536065 | AGENCOURT_10229596 NIH_MGC_141 Homo sapiens cDNA clone | ILMN_1881909 | TTCCAGGGCACGAGTTCGAGGCCAGCCTGGTCCACATG |
| | | IMAGE: 6563923 5, mRNA sequence | | GGTCGGaaaaaa |
| C1QL4 | NM_001008223.1 | Homo sapiens complement component 1, q subcomponent- | ILMN_1808117 | TAAAACAGGGTAGTGCAGGTTCTCCGTCACAACTTTCT |
| | | like 4 (C1QL4), mRNA. | | CTCGCCACCCTC |
| C9orf140 | NM_178448.2 | Homo sapiens chromosome 9 open reading frame 140 | ILMN_1702197 | AGCGTCCCTGGGCTCTATCCGCGAGGTGCCAGTAGCGT |
| | | (C9orf140), mRNA. | | GTGCAGGTACAT |
| CT45A4 | NM_001017436.1 | Homo sapiens cancer/testis antigen family 45, member A4 | ILMN_1672783 | AATGGAGCAGGATATTGCTGAAGTCTCCTGGCATATGT |
| | | (CT45A4), mRNA. | | TACCGAATCAAA |
| CXCL10 | NM_001565.2 | Homo sapiens chemokine (C-X-C motif) ligand 10 (CXCL10), | ILMN_1791759 | GACTTCCACTGCCATCCTCCCAAGGGGCCCAAATTCTT |
| | | mRNA. | | TCAGTGGCTACC |
| DLL3 | NM_016941.2 | Homo sapiens delta-like 3 (Drosophila) (DLL3), | ILMN_1736096 | TCCGCACCTGGAGTCAGAGCGTGGATTTTTGTATTTGC |
| | | transcript variant 1, mRNA. | | TCGGTGGTGCCC |
| KCNQ2 | NM_172109.1 | Homo sapiens potassium voltage-gated channel, KQT-like | ILMN_1666776 | ACCGCCGCCGGGCACCTGCCACCAAGCAACTGTTTCAT |
| | | subfamily, member 2 (KCNQ2), transcript variant 5, mRNA. | | TTTTTATTTTCC |
| LEMD1 | NM_001001552.3 | Homo sapiens LEM domain containing 1 (LEMD1), mRNA. | ILMN_1785444 | CGAGAGCTGGAGAGAAGAAGGTTTCCCAGTGGGCTTGA |
| | | | | AGCTTGCTGTGC |
| LOC645037 | NM_001098411.1 | Homo sapiens similar to GAGE-2 protein (G antigen 2) | ILMN_1674097 | TGTGAGGCAGTGCTGTGTGGTTCCTGCCGTCCGGACTC |
| | | (LOC645037), mRNA. | | TTTTTCCTCTAT |
| LOC647315 | XM_930384.1 | PREDICTED: Homo sapiens similar to microtubule- | ILMN_1804491 | GACGGCCAGCGAAAGATCTAGACCCAGCAGTTAGACGG |
| | | associated protein 6 isoform 1 (LOC647315), mRNA. | | CCAGCGAAAGAC |
| MMP11 | NM_005940.3 | Homo sapiens matrix metallopeptidase 11 (stromelysin 3), | ILMN_1655915 | CAGGTCTTGGTAGGTGCCTGCATCTGTCTGCCTTCTGG |
| | | (MMP11) mRNA. | | CTGACAATCCTG |
| NKX2-5 | NM_004387.2 | Homo sapiens NK2 transcription factor related, locus 5 | ILMN_1800058 | GGCTCCCAACATGACCCTGAGTCCCCTGGATTTTGCAT |
| | | (Drosophila) (NKX2-5), mRNA. | | TCACTCCTGCGG |
| PTHLH | NM_198965.1 | Homo sapiens parathyroid hormone-like hormone (PTHLH), | ILMN_2314169 | CCACCCCGTCCGATTTGGGTCTGATGATGAGGGCAGAT |
| | | transcript variant 1, mRNA. | | ACCTAACTCAGG |
| SALL4 | NM_020436.2 | Homo sapiens sal-like 4 (Drosophila) (SALL4), mRNA. | ILMN_1695687 | GCGGTCAGCTAAGGGAGAACTTGCGTGGAAGGAGCAAT |
| | | | | GCAGACACAGTG |
| SNORD56 | NR_002739.1 | Homo sapiens small nucleolar RNA, C/D box 56 (SNORD56), | ILMN_2209515 | TTCGTCAACAGCAGTTCACCTAGTGAGTGTTGAGACTC |
| | | small nuclear RNA. | | TGGGTCTGAGTG |
| CSAG3A | NM_203311.1 | Homo sapiens CSAG family, member 3A (CSAG3A), mRNA. | ILMN_2043126 | GCCACGAATAAGGCCATCACCAGAAGCCAACCCCGCCA |
| | | | | GTCCTTGATCTA |
| FAM83A | NM_207006.1 | Homo sapiens family with sequence similarity 83, member | ILMN_1670158 | CTGACCACCCTCCATCAGCAGTCTCCCCTCCGTGGTCG |
| | | A (FAM83A), transcript variant 2, mRNA. | | TCTTTGTTGACA |
| LOC100134331 | XM_001724554.1 | PREDICTED: Homo sapiens similar to hCG1812074 | ILMN_3243573 | GGAAGGGCCTGGAGTGGATTGGGTACATCTATTACAGT |
| | | (LOC100134331), mRNA. | | GGGAGCACCTAC |
| LOC642477 | XM_930694.1 | PREDICTED: Homo sapiens hypothetical protein LOC642477, | ILMN_1794711 | GTAGGAGGCAGGTCTCCGCGGTTCATCTGTGTTGCTCT |
| | | transcript variant 2 (LOC642477), mRNA. | | AAATGACACTGT |
| LOC645099 | XM_930411.1 | PREDICTED: Homo sapiens hypothetical protein LOC645099, | ILMN_1685016 | TTCAGATGGCACTTAAAGCAGAGAAGCCTGCTGTGTGG |
| | | transcript variant 1 (LOC645099), mRNA. | | CTGTGGGAGTCA |
| LOC729264 | XM_001133677.1 | PREDICTED: Homo sapiens similar to TP53TG3 protein, | ILMN_1744252 | TCACGTGTCTTCACGCATCTCTTGAATTGGAAATTGTG |
| | | transcript variant 2 (LOC729264), mRNA. | | CCCTGGAGACTG |
| PCDHB2 | NM_018936.2 | Homo sapiens protocadherin beta 2 (PCDHB2), mRNA. | ILMN_2227757 | TTGTGGAAAGTCCTTTTTTACTGCTTTGCCCATTGGAG |
| | | | | GTGTCTCCTTTT |
| PI3 | NM_002638.2 | Homo sapiens peptidase inhibitor 3, skin-derived | ILMN_1693192 | CTGACTGCCCAGGAATCAAGAAGTGCTGTGAAGGCTCT |
| | | (SKALP) (PI3), mRNA. | | TGCGGGATGGCC |
| TP53TG3 | NM_016212.2 | Homo sapiens TP53 target 3 (TP53TG3), mRNA. | ILMN_2159152 | TCACGTGTCTTCACGCATCCCTTGAATTGGAAATTGTG |
| | | | | CCCTGGAGACTG |
| CTSL2 | NM_001333.2 | Homo sapiens cathepsin L2 (CTSL2), mRNA. | ILMN_1748352 | GAGCTGATGGATGGTGAGGAGGAAGGACTTAAGGACAG |
| | | | | CATGTCTGGGGA |
| GREM1 | NM_013372.5 | Homo sapiens gremlin 1, cysteine knot superfamily, | ILMN_2124585 | CGGCAAAGAATTATATAGACTATGAGGTACCTTGCTGT |
| | | homolog (Xenopus laevis) (GREM1), mRNA. | | GTAGGAGGATGA |
| KCNK17 | NM_031460.3 | Homo sapiens potassium channel, subfamily K, member 17 | ILMN_1717702 | ACATGTCCTGGGTGACATGGGATGTGACTTTCGGGTGT |
| | | (KCNK17), transcript variant 1, mRNA. | | CGGGGCAGCATG |
| KREMEN2 | NM_024507.2 | Homo sapiens kringle containing transmembrane protein 2 | ILMN_2382290 | GGACCTGTATGTGGGGGTGGTCTCTGGTTTCGGAGGTC |
| | | (KREMEN2), transcript variant 2, mRNA. | | TTTGAACCCCTC |
| LOC100130082 | XM_001725008.1 | PREDICTED: Homo sapiens hypothetical protein | ILMN_3182981 | GTCCAGAGAGTCCAGGCTCATCATCCCTTCAGAAGAAA |
| | | LOC100130082, transcript variant 2 (LOC100130082), | | GAATCTTCAGGC |
| | | mRNA. | | |
| LOC645682 | XR_017655.1 | PREDICTED: Homo sapiens hypothetical LOC645682 | ILMN_1660709 | CAGGTTGGAGTGCGGCTAGTGCCCCAAGGCGGCTTGGA |
| | | (LOC645682), mRNA. | | GACCTCTCAGCC |
| OLFM4 | NM_006418.3 | Homo sapiens olfactomedin 4 (OLFM4), mRNA. | ILMN_2116877 | TGTTCAAGTCCTAGTCTATAGGATTGGCAGTTTAAATG |
| | | | | CTTTACTCCCCC |
| ONECUT2 | NM_004852.2 | Homo sapiens one cut homeobox 2 (ONECUT2), mRNA. | ILMN_1664462 | TTCTTCATGAACGCCCGGCGCCGCAGCCTGGAGAAGTG |
| | | | | GCAAGACGATCT |
| PPEF1 | NM_152224.1 | Homo sapiens protein phosphatase, EF-hand calcium | ILMN_1652017 | TGGGTTGGACCTAGTGGTGTTGTCGTGAGTGCCACCTA |
| | | binding domain 1(PPEF1), transcript variant 1b, mRNA. | | ACCAGGAGGCCA |
| RPRML | NM_203400.1 | Homo sapiens reprimo-like (RPRML), mRNA. | ILMN_1676504 | GCGTCAGAGTCGCTGAGCTTGTTGGCCTGATCTTGCGT |
| | | | | TTGGAAAGAAAT |
| WNT10A | NM_025216.2 | Homo sapiens wingless-type MMTV integration site family, | ILMN_1658426 | CCACACCCTAAAACAAGCCTCAGCCAGGCAACCCGTCA |
| | | member 10A (WNT10A), mRNA. | | GTCTGTCTCCAT |
| ANXA13 | NM_004306.2 | Homo sapiens annexin A13 (ANXA13), transcript variant | ILMN_2412490 | CAGGGCAATAGGAACACAGGGTGGAACCGCCTTTGTCA |
| | | 1, mRNA. | | AGAGCACATTCC |
| FLI22184 | NM_001080403.1 | Homo sapiens hypothetical protein FLI22184 (FLI22184), | ILMN_2387471 | TTGCCCACCCCGCACAGCCTGAGTTTGCAATAAAACTG |
| | | mRNA. | | GGACACTGGGAC |
| LAMC2 | NM_005562.1 | Homo sapiens laminin, gamma 2 (LAMC2), transcript | ILMN_1701424 | ACACCAGTGGGAATTGCTGGAGGAACCAGAGGCACTTC |
| | | variant 1, mRNA. | | CACCTTGGCTGG |
| MAPK15 | NM_139021.2 | Homo sapiens mitogen-activated protein kinase 15 | ILMN_1768506 | CTCCTGCACCCCTTAGCCCTCCCTGCTTTGCCTGGCCC |
| | | (MAPK15), mRNA. | | GTTGAAGTTCCA |
| NUP210 | NM_024923.2 | Homo sapiens nucleoporin 210 kDa (NUP210), mRNA. | ILMN_1784467 | TGGGGGAGGAGACCCTTGGAAAAGTCCTCTCTTCCCAG |
| | | | | CTCCTGATTCTG |
| ALG1L | NM_001015050.1 | Homo sapiens asparagine-linked glycosylation 1-like | ILMN_2131293 | CGGCAGTGGTGCCGCCTGGTGAATGAATTGGTTCTGTG |
| | | (ALG1L), mRNA. | | ACCCGGAAAAAA |
| GNG4 | NM_001098721.1 | Homo sapiens guanine nucleotide binding protein (G | ILMN_1804357 | CTGTAAAAGTACCCCATACCGTTGACGCGCTGTGGCAG |
| | | protein), gamma 4 (GNG4), transcript variant 2, mRNA. | | ACCTGTGGGTGC |
| HRK | NM_003806.1 | Homo sapiens harakiri, BCL2 interacting protein | ILMN_2193706 | AGCCCAGAGCTTGAAAGGCCGCGGTTGGCACTTCGAGA |
| | | (contains only BH3 domain) (HRK), mRNA. | | AGGAAGTGGAGA |
| NFE2L3 | NM_004289.5 | Homo sapiens nuclear factor (erythroid-derived 2)- | ILMN_2049766 | CCCAGTAAGACTTTCCATCTTGGCAGCCATCCTTTTTA |
| | | like 3 (NFE2L3), mRNA. | | AGAGTAAGTTGG |
| TET1 | NM_030625.2 | Homo sapiens tet oncogene 1 (TET1), mRNA. | ILMN_3247163 | CCCCACTGTGGGAACCAAATTGGATTCCTACTTTGTTG |
| | | | | GACTCTCTTTCC |
| 3-Sep | NM_019106.4 | Homo sapiens septin 3 (SEPT3), transcript variant B, | ILMN_1746673 | CCGTTTCTTAAATGTTACCAGTCCCAGCCAATCTTACG |
| | | mRNA. | | GTGACATTACAG |
| ASCL1 | NM_004316.2 | Homo sapiens achaete-scute complex homolog 1 | ILMN_1701653 | CTCCTCATAGGTGAGATCAAGAGGCCACCAGTTGTACT |
| | | (Drosophila) (ASCL1), mRNA. | | TCAGCACCAATG |
| BIK | NM_001197.3 | Homo sapiens BCL2-interacting killer (apoptosis- | ILMN_1770505 | CCATGACCACTGCCCTGGAGGTGGCGGCCTGCTGCTGT |
| | | inducing) (BIK), mRNA. | | TATCTTTTTAAC |
| C21orf129 | NM_152506.1 | Homo sapiens chromosome 21 open reading frame 129 | ILMN_2174711 | CTGTGTTTTCAGCCACCCAGTCTGTGGTATCTTGTGAC |
| | | (C21orf129), mRNA. | | TGCCGCCCTAGG |
| CAPN12 | NM_144691.3 | Homo sapiens calpain 12 (CAPN12), mRNA. | ILMN_2101034 | GCTAGCTCTGCCCTGGCTCTCCTAGAAGGTGGAGGACA |
| | | | | GACACAGGAGAA |
| CBX8 | NM_020649.1 | Homo sapiens chromobox homolog 8 (Pc class homolog, | ILMN_1775183 | TGTGTCCAGGAGGAGAGCAGGGGAGAGAGTGAGCGTGA |
| | | Drosophila) (CBX8), mRNA. | | GCTTGGCATAGT |
| CCL20 | NM_004591.1 | Homo sapiens chemokine (C-C motif) ligand 20 (CCL20), | ILMN_1657234 | CCTTGCTGGGGTTGGAGGTfTCACTTGCACATCATGGA |
| | | mRNA. | | GGGTTTAGTGCT |
| CGB5 | NM_033043.1 | Homo sapiens chorionic gonadotropin, beta polypeptide 5 | ILMN_2163790 | CGCCGTGGCTCTCAGCTGTCAATGTGCACTCTGCCGCC |
| | | (CGB5), mRNA. | | GCAGCACCACTG |
| CLDN9 | NM_020982.2 | Homo sapiens claudin 9 (CLDN9), mRNA. | ILMN_1740276 | CACCTCCCCAGTAATTGTTTCCTTCCGTTGCCCAGGAC |
| | | | | ACTGGCTGGCCT |
| CSAG1 | NM_153479.1 | Homo sapiens chondrosarcoma associated gene 1 (CSAG1), | ILMN_1737640 | AGGAGACCACCGCCTTCTCCAGTGCTTCCTTGGGCAGC |
| | | transcript variant b, mRNA. | | CAGTAATTCCCA |
| CSAG3B | NM_001080848.1 | Homo sapiens CSAG family, member 3B (CSAG3B), mRNA. | ILMN_2412880 | AAAAATGCACTGTGAGTTTCATGCCTGCTGGCCTGCCT |
| | | | | TCACTGTCCTGG |
| CT45A1 | NM_001017417.1 | Homo sapiens cancer/testis antigen family 45, member | ILMN_1679921 | GGAGATGACCTAGAATGCAGAGAAACAGCCTCCTCTCC |
| | | A1 (CT45A1), mRNA. | | CAAAAGCCAACG |
| CT45A5 | NM_001007551.2 | Homo sapiens cancer/testis antigen family 45, member A5 | ILMN_1670627 | GCAATTTCTCTGGAGATGACCTAGAATGCAGAGGAATA |
| | | (CT45A5), mRNA. | | GCCTCCTCTCCC |
| CTAG2 | NM_020994.2 | Homo sapiens cancer/testis antigen 2 (CTAG2), transcript | ILMN_2336585 | CAGTTGCACATCACGATGCCTTTCTCGTCGCCCATGGA |
| | | variant 2, mRNA. | | AGCGGAGCTGGT |
| CTCFL | NM_080618.2 | Homo sapiens CCCTC-binding factor (zinc finger pro- | ILMN_1745395 | GCCAGTTGACAAGATTTTTCCACCCTCGAGCAGCGTGA |
| | | tein)-like (CTCFL), mRNA. | | GAGATGCCTCTT |
| ERVK6 | NM_001007236.1 | Homo sapiens endogenous retroviral sequence K, 6 | ILMN_1787676 | AGAAAAGCACCTCCGCGGAGACGGAGACATCGCAATCG |
| | | (ERVK6), mRNA. | | AGCACCGTTGAC |
| FAM133A | NM_173698.1 | Homo sapiens family with sequence similarity 133, | ILMN_1781742 | CCAAATGGCATACTTACAAGACGGATGCAACCTGGGTC |
| | | member A (FAM133A), mRNA. | | CTTAGGTCGCTG |
| FLI39632 | XR_015133.1 | PREDICTED: Homo sapiens misc_RNA (FLI39632), miscRNA. | ILMN_1803559 | GGAGCAGCCACTGCAAATGCTGCGCTGACCCCAAATGC |
| | | | | TGTGTCCTTTAA |
| HIST1H3H | NM_003536.2 | Homo sapiens histone cluster 1, H3h (HIST1H3H), mRNA. | ILMN_1749368 | TCAAGAAGCCCCATCGCTATCGGCCTGGTACAGTGGCT |
| | | | | CTCCGCGAGATT |
| HIST1H4H | NM003543.3 | Homo sapiens histone cluster 1, H4h (HIST1H4H), mRNA. | ILMN_1751120 | CGCACTCTTTACGGCTTCGGTGGCTAAGGCTCCTGCTT |
| | | | | GCTGCACTCTTA |
| KIAA1199 | NM_018689.1 | Homo sapiens KIAA1199 (KIAA1199), mRNA. | ILMN_1813704 | GCAACGCTCCTCTGAAATGCTTGTCTTTTTTCTGTTGC |
| | | | | CGAAATAGCTGG |
| L1TD1 | NM_019079.2 | Homo sapiens LINE-1 type transposase domain containing | ILMN_1769839 | CTTCTACCCAGAAGGATGGACAGCTAATAGCGTACTTG |
| | | 1 (L1TD1), mRNA. | | GGGATGAGGAGC |
| LHX2 | NM_004789.3 | Homo sapiens LIM homeobox 2 (LHX2), mRNA. | ILMN_1807016 | AAGAAGTGTGCGCCCGGCTAATGCAGCGGTGTGGACCG |
| | | | | AGGAACAACTTG |
| LOC100132564 | XM_001713808.1 | PREDICTED: Homo sapiens hypothetical protein | ILMN_3243644 | GAGCAGCTCCCTCGCTGCGATCTATTGAAAGTCAGATC |
| | | LOC100132564 (LOC100132564), mRNA. | | TCCACACAAGGG |
| LOC400879 | XM_934985.1 | PREDICTED: Homo sapiens hypothetical LOC400879, | ILMN_1729197 | GTAGGAGGCAGGTCTCCGCGGTTCATCTGTGTTGCTCT |
| | | transcript variant 2 (LOC400879), mRNA. | | AAATGACACTGC |
| LOC643272 | XM_926633.1 | PREDICTED: Homo sapiens hypothetical protein LOC643272 | ILMN_1681260 | ATCTCTTGGTGCTATCCCCAAACTGCCACTCTTAATTC |
| | | (LOC643272), mRNA. | | CCTCTTAGAGTG |
| LOC653297 | XM_926730.1 | PREDICTED: Homo sapiens similar to CSAG family, member 2 | ILMN_1803852 | CCTCCAGCCCATTGTCCAACAACCACCCACCAACACCA |
| | | (LOC653297), mRNA. | | AAGAGGTTGCCA |
| LOC729669 | XM_001130489.1 | PREDICTED: Homo sapiens hypothetical LOC729669 | ILMN_3301763 | GGGAGAAGGTAGCTGTCGGGCATTCCCCTGGCGCTGAA |
| | | (LOC729669), mRNA. | | GGGCAGATTGCT |
| MSLN | NM_013404.3 | Homo sapiens mesothelin (MSLN), transcript variant 2, | ILMN_2353161 | TTCCACCCCAAGAGAACTCGCGCTCAGTAAACGGGAAC |
| | | mRNA. | | ATGCCCCCTGCA |
| NLRP7 | NM_206828.2 | Homo sapiens NLR family, pyrin domain containing 7 | ILMN_1658632 | CATTCCGAACTGGGCTCGGCAGGATCTTCGCTCTCTTC |
| | | (NLRP7), transcript variant 2, mRNA. | | GCCTCTGGACAG |
| ONECUT2 | NM_004852.2 | Homo sapiens one cut homeobox 2 (ONECUT2), mRNA. | ILMN_1838320 | CCTGTGAATACCTCAGCTTCAACTGGGCCTCCATACAG |
| | | | | TCAGTTGGTGGG |
| PCSK1 | NM_000439.3 | Homo sapiens proprotein convertase subtilisin/kexin | ILMN_2081813 | GTAGCTGAGTTTAACATGTGTGGTCTTGGTATTCTTAA |
| | | type 1 (PCSK1), mRNA. | | GGGAACTTCCAC |
| PDX1 | NM_000209.3 | Homo sapiens pancreatic and duodenal homeobox 1 (PDX1), | ILMN_3249216 | GCACAGTGGCCTGGTGCGCCTTGGAAACCAACAACTAT |
| | | mRNA. | | TCACGAGCCAGT |
| PSG1 | NM_006905.2 | Homo sapiens pregnancy specific beta-1-glycoprotein 1 , | ILMN_1798000 | GCAGGCAAAGTCTGAAGTCAGCCTTGGTTTGGCTTCCT |
| | | (PSG1) mRNA. | | ATTCTCAAGAGG |
| SERPINA1 | NM_000295.3 | Homo sapiens serpin peptidase inhibitor, clade A | ILMN_1764980 | AGTGGACTTAGCCCCTGTTTGCTCCTCCGATAACTGGG |
| | | (alpha-1 antiproteinase, antitrypsin), member 1 | | GTGACCTTGGTT |
| | | (SERPINA1), transcript variant 1, mRNA. | | |
| SYCP2 | NM_014258.2 | Homo sapiens synaptonemal complex protein 2 (SYCP2), | ILMN_2095704 | GGATGAGAGGGAACCACTATAACATGAGTCCAAGCCCA |
| | | mRNA. | | GAAGACTTCTGT |
| TDRD5 | NM_173533.2 | Homo sapiens tudor domain containing 5 (TDRD5), mRNA. | ILMN_1700887 | CAGAATCCAGCCGCTTAGGCTTTGATGAACTCCCAGGC |
| | | | | CAAAATGAGGAG |
| UTS2D | NM_198152.2 | Homo sapiens urotensin 2 domain containing (UTS2D), | ILMN_2180232 | GCTGGTATATCCAGTGCATTGTTGGCACCATGGGACCA |
| | | mRNA. | | GAAGGTGGTGAC |
| WDR66 | NM_144668.4 | Homo sapiens WD repeat domain 66 (WDR66), mRNA. | ILMN_1800341 | TCCCGAGGGATGGAAATCCGAGCCTGCAACCTGCTCCG |
| | | | | TCAAAGGTTCAG |
| XAGE1B | NM_001097595.1 | Homo sapiens X antigen family, member 1B (XAGE1B), | ILMN_1691494 | TGCGCGACATGGAAGGTGATCTGCAAGAGCTGCATCAG |
| | | transcript variant 1, mRNA. | | TCAAACACCGGG |
| CT0321 | AW578902 | RC2-CT0321-110100-013-c08 CT0321 Homo sapiens cDNA, | ILMN_1832656 | TGGGGAGAGCACAAGAGAGCCGTGACAGAGGAAGGGAG |
| | | mRNA sequence | | AGAGCACAGAGT |
| MSH5 | NM_002441.3 | Homo sapiens mutS homolog 5 (E. coli) (MSH5), tran- | ILMN_1780292 | AATTTGGAAAGGGAACCAACACGGTGGATGGGCTCGCG |
| | | script variant 3, mRNA. | | CTTCTGGCCGCT |
| MTBP | NM_022045.3 | Homo sapiens Mdm2, transformed 3T3 cell double minute | ILMN_1660222 | CCGAGACTCATGAATGTTTCACTGCATGCAGCCAGCGT |
| | | 2, p53 binding protein (mouse) binding protein, 104 kDa | | CTCTTTGAAATC |
| | | (MTBP), mRNA. | | |
| COL11A1 | NM_001854.3 | Homo sapiens collagen, type XI, alpha 1 (COL11A1), | ILMN_1789507 | GGTGCCACCAACCCATTTTGTGCCACATGCAAGTTTTG |
| | | transcript variant A, mRNA. | | AATAAGGATGGT |
| DOK7 | NM_173660.3 | Homo sapiens docking protein 7 (DOK7), mRNA. | ILMN_1654212 | AGGAAACTGAAGCTCAGGAGGCTGTGTGGCTTGCGGGG |
| | | | | TCTCTGGGTTCT |
| FGF11 | NM_004112.2 | Homo sapiens fibroblast growth factor 11 (FGF11), mRNA. | ILMN_1719938 | AGGAGTAGATGCCCCCTCACCCACACAAACCCCACTCA |
| | | | | GTCTCCACCCAA |
| GAD1 | NM_013445.3 | Homo sapiens glutamate decarboxylase 1 (brain, 67 kDa) | ILMN_1660973 | TGCACACATGGTTTCCAAGGGTCTTCCTCCTAAATTTC |
| | | (GAD1), transcript variant GAD25, mRNA. | | CAGGGGCCTCCC |
| HORMAD1 | NM_032132.3 | Homo sapiens HORMA domain containing 1 (HORMAD1), mRNA. | ILMN_1769849 | TGTTCTGCAGGCTTGCAGAGTTCTTCTCACCATTTAAA |
| | | | | CTGAAGGACCCT |
| MAGEA12 | NM_005367.4 | Homo sapiens melanoma antigen family A, 12 (MAGEA12), | ILMN_2231003 | GTGTGACATGAGGCCCATTCTTCACTCTTTGAAGAGAG |
| | | mRNA. | | CAGTCAGTATTG |
| MMP7 | NM_002423.3 | Homo sapiens matrix metallopeptidase 7 (matrilysin, | ILMN_1685403 | GCTCACTTCGATGAGGATGAACGCTGGACGGATGGTAG |
| | | uterine) (MMP7), mRNA. | | CAGTCTAGGGAT |
| NLRP7 | NM_139176.2 | Homo sapiens NLR family, pyrin domain containing 7 | ILMN_1652366 | GCAGCCTGGGATCGCTCTACGAATTACACAGGAAGCGG |
| | | (NLRP7), transcript variant 1, mRNA. | | GATTCGGGTCTC |
| NSUN5 | NM_018044.2 | Homo sapiens NOL1/NOP2/Sun domain family, member 5 | ILMN_2408400 | ACGTGCTCCCTCTGCCAGGAGGAGAATGAAGACGTGGT |
| | | (NSUN5), transcript variant 2, mRNA. | | GCGAGATGCGCT |
| TBX1 | NM_005992.1 | Homo sapiens T-box 1 (TBX1), transcript variant B, mRNA. | ILMN_2350514 | GGTCACTGCCTACCAGAACCATCGGATCACGCAGCTCA |
| | | | | AGATTGCCAGCA |
| TNFRSF6B | NM_003823.2 | Homo sapiens tumor necrosis factor receptor super- | ILMN_1661825 | AAGGAGGTGGCATGTCGGTCAGGCACAGCAGGGTCCTG |
| | | family, member 6b, decoy (TNFRSF6B), transcript variant | | TGTCCGCGCTGA |
| | | M68E, mRNA. | | |
| UGT1A6 | NM_205862.1 | Homo sapiens UDP glucuronosyltransferase 1 family, | ILMN_1752813 | TACCAGGCTTTCTGACTCCTGCTCTAGGATTCTCACCA |
| | | polypeptide A6 (UGT1A6), transcript variant 2, mRNA. | | CGTACTGGCTAG |
| ZNF280A | NM_080740.3 | Homo sapiens zinc finger protein 280A (ZNF280A), mRNA. | ILMN_1802094 | GACTTCCAGGAGTTCCGAAAGGCAGAAGTGAGGCTAGG |
| | | | | CCATATGTGCAT |
| EPYC | NM_004950.3 | Homo sapiens epiphycan (EPYC), mRNA. | ILMN_1677567 | CCACATCCCTCTGCCACTCCCAGAAAATCTACGAGCCC |
| | | | | TTCACCTCCAGA |
| NMU | NM_006681.1 | Homo sapiens neuromedin U (NMU), mRNA. | ILMN_2162253 | GCTGCAGCTCGTTCCTCACCTGCATGAGAGAAGAATGA |
| | | | | AGAGATTCAGAG |
| SPRYD5 | NM_032681.1 | Homo sapiens SPRY domain containing 5 (SP RYD5), mRNA. | ILMN_1753648 | TCCCTGATATACACCATCCCCAATTGCTCCTTCTCACC |
| | | | | TCCTCTCAGGCC |
| VCX2 | NM_016378.2 | Homo sapiens variable charge, X-linked 2 (VCX2), mRNA. | ILMN_1651789 | CGACCGTTGCGAGACGTTGAGCTGCGGAAGATGAGTCC |
| | | | | AAAGCCGAGAGC |
| GRN_ES | CN304251 | 17000532640995 GRN_ES Homo sapiens cDNA 5, mRNA sequence | ILMN_1904785 | GGCCGTAGCTGCCTTGCGGATTGTTACGTTGTCCAGCT |
| | | | | TGTCTCCACACG |
| LOC651957 | XM_945048.1 | PREDICTED: Homo sapiens hypothetical protein LOC651957 | ILMN_1737110 | GAGAACATCACCACCCTGAAGCCAGAGACTAACACTGC |
| | | (LOC651957), mRNA. | | AGGACTCAGCAA |
| VCX3A | NM_016379.2 | Homo sapiens variable charge, X-linked 3A (VCX3A), mRNA. | ILMN_2366642 | GCCAGGTGGAGGAACCACTGAGTCAGGAGAGCGAGATG |
| | | | | GAAGAACTACCG |
| CXCR3 | NM_001504.1 | Homo sapiens chemokine (C-X-C motif) receptor 3 | ILMN_1797975 | ACTTCATCTTCCCCAAGTGCGGGGAGTACAAGGCATGG |
| | | (CXCR3), transcript variant A, mRNA. | | CGTAGAGGGTGC |
| HIST1H2AM | NM_003514.2 | Homo sapiens histone cluster 1, H2am (HIST1H2AM), mRNA. | ILMN_1756022 | AACCCGCTCCTCTAGAGCTGGGCTCCAATTTCCTGTAG |
| | | | | GACGAGTGCACC |
| KIF24 | NM_194313.2 | Homo sapiens kinesin family member 24 (KIF24), mRNA. | ILMN_1694126 | GCCTATCCCAACTCCACAGTCAGGAAGGCCTACGTCCT |
| | | | | TGGTCCACAGAC |
| C3orf32 | NM_015931.1 | Homo sapiens chromosome 3 open reading frame 32 | ILMN_1666731 | ACCAGGTGTATGCGGTGGACTATCCTGAGCGGTATTGC |
| | | (C3orf32), mRNA. | | TGTGGCTGTACC |
| IL8 | NM_000584.2 | Homo sapiens interleukin 8 (IL8), mRNA. | ILMN_1666733 | CCCTAGTCTGCTAGCCAGGATCCACAAGTCCTTGTTCC |
| | | | | ACTGTGCCTTGG |
| SNORA72 | NR_002581.1 | Homo sapiens small nucleolar RNA, H/ACA box 72 | ILMN_3240418 | GACCATGCATGTGTCCCCAAACCTAGTTCTTTCCCTAG |
| | | (SNORA72), small nucleolar RNA. | | GTCTGGTTTCAT |
| NTS | NM_006183.3 | Homo sapiens neurotensin (NTS), mRNA. | ILMN_1764690 | CCACAAAATCTGTCACAGCAGGGCTTTTCAACACTGGG |
| | | | | AGTTAATCCAGG |
| PPM1E | NM_014906.3 | Homo sapiens protein phosphatase 1E (PP2C domain | ILMN_1708508 | CTCTTACTCTAGGTGCTCTTTGGTGAGAGACAGGCTTT |
| | | containing) (PPM1E), mRNA. | | GTTCTCTTGTTC |
| TM4SF19 | XM_001134247.1 | PREDICTED: Homo sapiens transmembrane 4 L six family | ILMN_1808325 | GCCCTTCTGTGCATCAGCCTGCTCCAGCTTCTCCTGGT |
| | | member 19, transcript variant 2 (TM4SF19), mRNA. | | GGTCGTTCATGT |
| BIRC7 | NM_022161.2 | Homo sapiens baculoviral IAP repeat-containing 7 | ILMN_2338849 | AGTTGCGTCTGGCCTCCTTCTATGACTGGCCGCTGACT |
| | | (BIRC7), transcript variant 2, mRNA. | | GCTGAGGTGCCA |
| NXPH4 | XM_938935.2 | PREDICTED: Homo sapiens neurexophilin 4 (NXPH4), mRNA. | ILMN_1741214 | CTCCCACCATTCTGCCTGCCATATGCCTGTCCCCTTTT |
| | | | | CCTCCAAACCCT |
| ANXA13 | NM_004306.2 | Homo sapiens annexin A13 (ANXA13), transcript variant | ILMN_1799243 | GAGTCCCGGATTACTTTCTTGGCAGCTTAAGTGGCGCA |
| | | 1, mRNA. | | GCCAGGCCAAGC |
| APOBEC1 | NM_001644.3 | Homo sapiens apolipoprotein B mRNA editing enzyme, | ILMN_1813881 | GCTGGAGGAATTTTGTCAACTACCCACCTGGGGATGAA |
| | | catalytic polypeptide 1 (APOBEC1), mRNA. | | GCTCACTGGCCA |
| C1orf110 | NM_178550.3 | Homo sapiens chromosome 1 open reading frame 110 | ILMN_1656088 | GTCAGCAGCTCTATCTCACCAATGACAGCCAGAATAGC |
| | | (C1orf110), mRNA. | | AAGCAACCACTG |
| C1QTNF3 | NM_030945.2 | Homo sapiens C1q and tumor necrosis factor related | ILMN_1768925 | GATGATGTGAACAGCCATGTGAATAGGTGACTTGGGCA |
| | | protein 3 (C1QTNF3), transcript variant 1, mRNA. | | CACAGCAGGGTC |
| CD70 | NM_001252.3 | Homo sapiens CD70 molecule (CD70), mRNA. | ILMN_1760247 | GAGGGGACACACTCTGCACCAACCTCACTGGGACACTT |
| | | | | TTGCCTTCCCGA |
| COX7B2 | NM_130902.2 | Homo sapiens cytochrome c oxidase subunit VIIb2 | ILMN_1674658 | CCAGTAGCTGAAGGCAACTGCAATCCTTCATGATGTTT |
| | | (COX7B2), mRNA. | | CCCTTGGCCAGA |
| GAGE12B | NM_001127345.1 | Homo sapiens G antigen 126 (GAGE12B), mRNA. | ILMN_3243856 | CGGCCCGAGCAGTTCAGTGATGAAGTGGAACCAGCAAC |
| | | | | ACCTGAAGAAGG |
| GAGE12G | NM_001098409.1 | Homo sapiens G antigen 12G (GAGE12G), mRNA. | ILMN_1664660 | ACGCCAGGGAGCTGTGAGGCAGTGCTGTGTGGTTCCTG |
| | | | | CCGTCCGGACTC |
| GAPDHS | NM_014364.3 | Homo sapiens glyceraldehyde-3-phosphate dehydrogenase, | ILMN_1794117 | GGCGCCCCACGCCGATGGGTCCATGGTGAAATAAAAAA |
| | | spermatogenic (GAPDHS), mRNA. | | CAGTGCTCGAAA |
| GTSF1 | NM_144594.1 | Homo sapiens gametocyte specific factor 1 (GTSF1), mRNA. | ILMN_2069632 | GGGGCACAACTCACTACTCTGACAACAACAGCCCTGCG |
| | | | | AGCAACATAGTT |
| HIST1H2BJ | NM_021058.3 | Homo sapiens histone cluster 1, H2bj (HIST1H2BJ), mRNA. | ILMN_1658702 | TTTATAGCTACACAGTGCTATGCCAGAGCCAGCGAAGT |
| | | | | CTGCTCCCGCCC |
| HIST2H4A | NM_003548.2 | Homo sapiens histone cluster 2, H4a (HIST2H4A), mRNA. | ILMN_2115340 | GCCGCTCCAGCTTTGCACGTTTCGATCCCAAAGGCCCT |
| | | | | TTTTAGGGCCGA |
| INA | NM_032727.2 | Homo sapiens internexin neuronal intermediate filament | ILMN_1673704 | GGACAGTCAGCTCTTCATCTGCCCAACTGTGTAGCATC |
| | | protein, alpha (INA), mRNA. | | TGCATTGCCCAG |
| KCNH6 | NM_173092.1 | Homo sapiens potassium voltage-gated channel, subfamily | ILMN_1677815 | ACATCCCCTGGAAGTACAAGGACTCATCTGTGGTCCCT |
| | | H (eag-related), member 6 (KCNH6), transcript variant | | GCTTCTCCTCCC |
| | | 2, mRNA. | | |
| KCNMB2 | NM_005832.3 | Homo sapiens potassium large conductance calcium- | ILMN_1687331 | AACTGAGAGAAAGAGCAACAAAGCGGCGAGTGGTGTGA |
| | | activated channel, subfamily M, beta member 2 (KCNMB2), | | GAGGGCAGCACG |
| | | transcript variant 2, mRNA. | | |
| KIAA1688 | NM_025251.1 | Homo sapiens KIAA1688 protein (KIAA1688), mRNA. | ILMN_1784436 | CCCGACGCATGGACCCGAGAGGCGACGACACGAGTGAA |
| | | | | TAAAGTGCACAT |
| LHX8 | NM_001001933.1 | Homo sapiens LIM homeobox 8 (LHX8), mRNA. | ILMN_1794818 | GGCTTATTCTGCCTACGTGCCCCAAGATGGAACGATGT |
| | | | | TAACTGCGCTGC |
| LOC100131707 | XR_038151.1 | PREDTCTED: Homo sapiens misc_RNA (LOC100131707), | ILMN_3245738 | AGCATTTGGGTGAAGACGGAGGTGGGTTCTGGACAGAC |
| | | miscRNA. | | CTACGCTGTCAG |
| LOC100133312 | XR_038222.1 | PREDTCTED: Homo sapiens misc_RNA (LOC100133312), | ILMN_3205264 | TTCTGGACCTCAGTCCTTCACCTAGTCACCTAGTCACA |
| | | miscRNA. | | GGGTGGATCGCC |
| LOC100133542 | XM_001719794.1 | PREDTCTED: Homo sapiens hypothetical protein | ILMN_3236833 | ACTAACGAGGACGCCGTCCAGGGCATCGCTAACGAGGA |
| | | LOC100133542 (LOC100133542), partial mRNA. | | CGCCGTCCACAG |
| LOC100134794 | XM_001718809.1 | PREDTCTED: Homo sapiens similar to keratin 8 | ILMN_3247286 | AGTCACGGTCAATCAACCAGAGCCTGCTGAGCCCCCTT |
| | | (LOC100134794), mRNA. | | ATCCTAGAGGTG |
| LOC651397 | XR_037048.1 | PREDTCTED: Homo sapiens misc_RNA (LOC651397), miscRNA. | ILMN_3242166 | GTTAGGAATGAAAGCATCCCTGGAGAACAGCCTGGAGG |
| | | | | AGACCAAAGGCC |
| LOC728178 | XR_040765.1 | PREDTCTED: Homo sapiens misc_RNA (LOC728178), miscRNA. | ILMN_3277072 | CCGAAGAAGGTTGGCCCTGCCAAAAACCTGATTTCAGA |
| | | | | CTTCTAGCCCCC |
| MAGEA1 | NM_004988.3 | Homo sapiens melanoma antigen family A, 1 (directs | ILMN_2181593 | GCGGTCAGTGTTCTCAGTAGTAGGTTTCTGTTCTATTG |
| | | expression of antigen MZ2-E) (MAGEA1), mRNA. | | GGTGACTTGGAG |
| MAGEA4 | NM_002362.4 | Homo sapiens melanoma antigen family A, 4 (MAGEA4), | ILMN_2361714 | GCCAGTGCATCTAACAGCCCTGTGCAGCAGCTTCCCTT |
| | | transcript variant 2, mRNA. | | GCCTCGTGTAAC |
| MAGEA6 | NM_175868.1 | Homo sapiens melanoma antigen family A, 6 (MAGEA6), | ILMN_1675651 | TCCTGCATGAGTGGGCTTTGAGAGAGGGGGAAGAGTGA |
| | | transcript variant 2, mRNA. | | GTCTGAGCACGA |
| MAGEB2 | NM_002364.3 | Homo sapiens melanoma antigen family B, 2 (MAGEB2), | ILMN_1688335 | CCAAAGCCAAGTTTACCTGCTGTTCTCACCCCCAATGA |
| | | mRNA. | | GGTCTTAGGCAG |
| MAGEC1 | NM_005462.3 | Homo sapiens melanoma antigen family C, 1 (MAGEC1), | ILMN_2241627 | CACACCCAAACACACCACATTGGGAAAACCTTCTGCCT |
| | | mRNA. | | CATTTTGTGATG |
| MAGEC2 | NM_016249.2 | Homo sapiens melanoma antigen family C, 2 (MAGEC2), | ILMN_2088876 | TAGTGGAACAAAATTGAAGGGTGGTCAGTAGTTTCATT |
| | | mRNA. | | TCCTTGTCCTGC |
| MAP1LC3A | NM_181509.1 | Homo sapiens microtubule-associated protein 1 light | ILMN_1711986 | CCGAGTTGCTGACTGACCCTCCACCTCAGAGGTAGTTC |
| | | chain 3 alpha (MAP1LC3A), transcript variant 2, mRNA, | | TGACACTGTCTC |
| MAP4K1 | NM_001042600.1 | Homo sapiens mitogen-activated protein kinase kinase | ILMN_2365111 | TGGAGGCCGTGGCTATGGTTGGAGGTCAGCTTCAGGCC |
| | | kinase kinase 1 (MAP4K1), transcript variant 1, mRNA. | | TTCTGGAAGCAT |
| MIR25 | NR_029498.1 | Homo sapiens microRNA 25 (MIR25), microRNA. | ILMN_3309534 | TTGGGCAATTGCTGGACGCTGCCCTGGGCATTGCACTT |
| | | | | GTCTCGGTCTGA |
| MTL5 | NM_004923.3 | Homo sapiens metallothionein-like 5, testis-specific | ILMN_1661778 | AGATATTTCCCCAGAGGCACGCGAACTGTCAGTCTTTC |
| | | (tesmin) (MTL5), transcript variant 1, mRNA. | | CTAAGGCCCCCG |
| NDUFA4L2 | NM_020142.3 | Homo sapiens NADH dehydrogenase (ubiquinone) 1 alpha | ILMN_1756573 | TACGTGTTGAGCGTGGCCTACGTGAGCCAACAAGAAGC |
| | | subcomplex, 4-like 2 (NDUFA4L2), mRNA. | | AGGGGCCTCTGA |
| NLRP7 | NM_139176.2 | Homo sapiens NLR family, pyrin domain containing 7 | ILMN_1798063 | TTGGATCTGCTCTCCTCAGCAATCAGAAGCTTGAAACT |
| | | (NLRP7), transcript variant 1, mRNA. | | CTGGACCTGGGC |
| NSUN5C | NM_032158.3 | Homo sapiens NOP2/Sun domain family, member 5C (NSUN5C), | ILMN_1718449 | CTCGGGTATGCCGAGCAGACAGCTGGAGGAGCCCGGGG |
| | | transcript variant 1, mRNA. | | CAGGGACACCTA |
| OBP2B | NM_014581.2 | Homo sapiens odorant binding protein 2B (OBP2B), mRNA. | ILMN_1700666 | GCCCAGTGACCTGCCGAGGTCGGCAGCACAGAGCTCTG |
| | | | | GAGATGAAGACC |
| PAGE2 | NM_207339.2 | Homo sapiens P antigen family, member 2 (prostate | ILMN_1724213 | GCAGTGCCTGCTTTTCAAGGGCCTGACATGGAAGCTTT |
| | | associated) (PAGE2), mRNA. | | TCAACAGGAACT |
| PAGE5 | NM_130467.3 | Homo sapiens P antigen family, member 5 (prostate | ILMN_2363141 | GGGACTCTGCCCACTTTTGATCCCACTAAAGTGCTGGA |
| | | associated) (PAGE5), transcript variant 1, mRNA. | | AGCAGGTGAAGG |
| PCLO | NM_033026.5 | Homo sapiens piccolo (presynaptic cytomatrix protein) | ILMN_3230160 | TGGACCATCTCGCAGTCAAAGCAAAACCAGCGTCACTC |
| | | (PCLO), transcript variant 1, mRNA. | | AGACCCACCTGG |
| PIWIL1 | NM_004764.3 | Homo sapiens piwi-like 1 (Drosophila) (PIWIL1), mRNA. | ILMN_1701220 | AGAGGCGTAAAGTAGGATGCTCACTACAACCATAGGTG |
| | | | | GGGTTTCAGCTC |
| PODXL2 | NM_015720.1 | Homo sapiens podocalyxin-like 2 (PODXL2), mRNA. | ILMN_1657347 | TTCCCGCTTCCCCCGACTTCACACGGCGGCTTCGGACC |
| | | | | AACTCCCTCACT |
| PRND | NM_012409.2 | Homo sapiens prion protein 2 (dublet) (PRND), mRNA. | ILMN_1684795 | CATTGCCTTGTTGATGGGCCTTCAGATTATTTCCAGTT |
| | | | | TTTTGCCACTGC |
| SLC45A2 | NM_016180.3 | Homo sapiens solute carrier family 45, member 2 | ILMN_2246188 | CGTCTGCGGTGGCACTGATAGGCTGTTGCTTTGTCGCT |
| | | (SLC45A2), transcript variant 1, mRNA. | | CTCTTTGTTAGA |
| SNORD3A | NR_006880.1 | Homo sapiens small nucleolar RNA, C/D box 3A (SNORD3A), | ILMN_3239574 | CTGCAACTGCCGTCAGCCATTGATGATCGTTCTTCTCT |
| | | small nucleolar RNA. | | CCGTATTGGGGA |
| SNORD3C | NR_006881.1 | Homo sapiens small nucleolar RNA, C/D box 3C (SNORD3C), | ILMN_3241034 | GAAGCCGGCTTTCTGGCGTTGCTTGGCTGCAACTGCCG |
| | | small nucleolar RNA. | | TCAGCCATTGAT |
| SNORD3D | NR_006882.1 | Homo sapiens small nucleolar RNA, C/D box 3D (SNORD3D), | ILMN_3242315 | GTAGAGCACCGAAAACCCCGAGGAAGAGAGGTAGCGTT |
| | | small nucleolar RNA. | | TTCTCCTGAGCG |
| SUNC1 | NM_001030019.1 | Homo sapiens Sad1 and UNC84 domain containing 1 (SUNC1), | ILMN_1657847 | GGGTCCATGGCACACCAGGCAAGCACATCTAGAAGAGT |
| | | transcript variant 1, mRNA. | | TGGTACAGAAGG |
| SYT13 | NM_020826.1 | Homo sapiens synaptotagmin XIII (SYT13), mRNA. | ILMN_1658499 | CTGGGCACAGAGAATCAGCTAGGAGACCAGTTATTCAG |
| | | | | GGTCCATTTCTC |
| TRIML2 | NM_173553.1 | Homo sapiens tripartite motif family-like 2 (TRIML2), | ILMN_1666893 | CTCCCATTGCGCCTTCCAAGGAGCTCTCAGGCCTGTGT |
| | | mRNA. | | TTTCCCTCTGTA |
| TRPM2 | NM_001001188.3 | Homo sapiens transient receptor potential cation | ILMN_2352380 | AGGAGGGGAACGTGGTAAAACCCAAGACATTAAATCTG |
| | | channel, subfamily M, member 2 (TRPM2), transcript | | CCATCTCAGGCC |
| | | variant S, mRNA. | | |
| TUBB3 | NM_006086.2 | Homo sapiens tubulin, beta 3 (TUBB3), mRNA. | ILMN_1791726 | TCCTCCCCACCTAGGCCACGTGTGAGCTGCTCCTGTCT |
| | | | | CTGTCTTATTGC |
| UCA1 | NR_015379.2 | Homo sapiens urothelial cancer associated 1 (non- | ILMN_3239254 | TCCTCGGCTTAGTGGCTGAAGACTGATGCTGCCCGATC |
| | | protein coding) (UCA1), non-coding RNA. | | GCCTCAGAAGCC |
| VCX | NM_013452.2 | Homo sapiens variable charge, X-linked (VCX), mRNA. | ILMN_1684886 | GAACCACTGAGTCAGGAGAGCCAGGTGGAGGAACCACC |
| | | | | GAGTCAGGAGAG |
| VCX-C | NM_001001888.1 | Homo sapiens variably charged X-C (VCX-C), mRNA. | ILMN_2166716 | GGTGGAGGAACCACTGAGTCAGGAGAGCGAGATGGAAG |
| | | | | AACCACTGAGTC |
| VCX2 | NM_016378.2 | Homo sapiens variable charge, X-linked 2 (VCX2), mRNA. | ILMN_2378845 | ATGAGTCCAAAGCCGAGAGCCTCGGGACCTCCGGCCAA |
| | | | | GGCCACGGAGGC |
| VCY | NM_004679.2 | Homo sapiens variable charge, Y-linked (VCY), mRNA. | ILMN_1683872 | CCTGGAGTTAGTCGACCGTTGCGAGACGTTGAGCTGCG |
| | | | | GCAGATGAGTCC |
| VGF | NM_003378.2 | Homo sapiens VGF nerve growth factor inducible (VGF), | ILMN_1757497 | TAATTGTGTGAAGTGTGTCTGTCTCCAGCCCTTCGGGC |
| | | mRNA. | | CTCCCACGAGCC |
| XAGE1 | NM_133431.1 | Homo sapiens X antigen family, member 1 (XAGE1), | ILMN_2343774 | GGAACGCGGCGGAGCTGTGAGCCGGCGACTCGGGTCCC |
| | | transcript variant 2, mRNA. | | TGAGGTCTGGAT |
| HESC3_16_C05.g1_A036 | CX782759 | HESC3_16_C05.g1_A036 Human embryonic stem cells Homo | ILMN_1837167 | TGGACTTCAATCCGGCCTCCCACATTATTCCTGATACC |
| | | sapiens cDNA clone IMAGE: 7476876 5, mRNA sequence | | GCACCTGACCCC |

1. A method of detecting cancer in a sample comprising a) contacting the sample with one or more agents that detect expression of at least one of the markers encoded for by the genes chosen from SLC35D, NMU, MMP12, MMP11, MMP7, DSCR8, COL10A, C2orf70, C12orf56, ASCL1, WNT10A, OLFM4, PI3, IL8, EPYC, and CXCL10; c) contacting a non-cancerous cell, with the one or more agents from b); and d) comparing the expression level of one or more of the markers encoded for by the genes chosen from GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the sample with the expression level of one or more of the markers chosen from GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the non-cancerous cell, wherein a higher level of expression in the sample of one or more of the markers encoded for by the genes chosen from GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the sample compared to the non-cancerous cell indicates that the sample has cancer cells.

2. The method of claim 1, wherein the sample is obtained from a subject.

3. The method of claim 2, wherein the subject is a human.

4. The method of claim 3, wherein the sample is a bodily fluid.

5. The method of claim 4, wherein the bodily fluid is serum.

6. The method of claim 1, wherein the agent is a protein.

7. The method of claim 6, wherein the agent is an antibody.

8. The method of claim 1, wherein the agent is a nucleic acid.

9. The method of claim 8, wherein the nucleic acid is a DNA molecule.

10. The method of claim 8, wherein the nucleic acid molecule is about 10-500 nucleotides in length.

11. The method of claim 1, wherein the agent has a detectible substance linked to it.

12. The method of claim 1, wherein the cancer is chosen from lung cancer, breast cancer, colon cancer, bladder cancer, kidney cancer and pancreatic cancer.

13. The method of claim 1 comprising a) contacting the sample with one or more agents that detect expression of the markers encoded for by the genes SLC35D, NMU, MMP12, MMP11, MMP7, DSCR8, COL10A, C2orf70, C12orf56, ASCL1, WNT10A, OLFM4, PI3, IL8, EPYC, AND CXCL10; c) contacting a non-cancerous cell, with the one or more agents from b); and d) comparing the expression level of the markers encoded for by the genes GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the sample with the expression level of the markers GNGT1, C12orf56, COL10A1, SLC35D3, snaR-A, SBK1, DSCR8, CELSR3 SLC35D, NMU, MMP12, MMP11, MMP7, DSCR8, COL10A, C2orf70, C12orf56, ASCL1, WNT10A, OLFM4, PI3, IL8, EPYC, and CXCL10 in the non-cancerous cell, wherein a higher level of expression of at least one of the markers in the sample compared to the non-cancerous cell indicates that the sample has cancer cells.

14. A kit for detecting cancer in a sample comprising a plurality of agents that specifically bind to a molecule encoded for by the genes SLC35D, NMU, MMP12, MPH, MMP7, DSCR8, COL10A, C2orf70, C12orf56, ASCL1, WNT10A, OLFM4, PI3, IL8, EPYC, and CXCL10.

15. The kit of claim 14, wherein the agents are nucleic acid molecules.

16. The kit of claim 15, wherein the nucleic acid molecules are DNA molecules.

17. The kit of claim 14, wherein the agents are proteins.

18. The kit of claim 17, wherein the proteins are antibodies.

19. The kit of claim 14, wherein the agents are labeled with a detectible substance.

20. A method of detecting cancer in a subject comprising a) obtaining a sample from a subject b) contacting the sample obtained from the subject with one or more agents that detect expression of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (P13), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1 orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof; c) contacting a non-cancerous cell with the one or more agents from b); and d) comparing the expression level of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens HORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1 orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (HIST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof in the non-cancerous cell, wherein a higher level of expression of one or more of the markers encoded by genes chosen from Homo sapiens preferentially expressed antigen in melanoma (PRAME), Homo sapiens anti-Mullerian hormone (AMH), Homo sapiens chromosome 12 open reading frame 56 (C12orf56), Homo sapiens Down syndrome critical region gene 6 (DSCR6), Homo sapiens guanine nucleotide binding protein (G protein), gamma transducing activity polypeptide 1 (GNGT1), Homo sapiens solute carrier family 35, member D3 (SLC35D3), Homo sapiens chromosome 2 open reading frame 70 (C2orf70), Homo sapiens cadherin, EGF LAG seven-pass G-type receptor 3 (flamingo homolog, Drosophila) (CELSR3), Homo sapiens collagen, type X, alpha 1 (COL10A1), Homo sapiens Down syndrome critical region gene 8 (DSCR8), transcript variant 2, Homo sapiens lin-28 homolog B (C. elegans) (LIN28B), Homo sapiens mesoderm specific transcript homolog (mouse) (MEST), transcript variant 2, Homo sapiens matrix metallopeptidase 12 (macrophage elastase) (MMP12), Homo sapiens SH3-binding domain kinase 1 (SBK1), AGENCOURT_{—}10229596 NIH_MGC_{—}141 Homo sapiens cDNA clone IMAGE:6563923 5, Homo sapiens complement component 1, q subcomponent-like 4 (C1QL4), mRNA, Homo sapiens chromosome 9 open reading frame 140 (C9orf140), Homo sapiens cancer/testis antigen family 45, member A4 (CT45A4), Homo sapiens chemokine (C—X—C motif) ligand 10 (CXCL10), Homo sapiens delta-like 3 (Drosophila) (DLL3), Homo sapiens potassium voltage-gated channel, KQT-like subfamily, member 2 (KCNQ2), Homo sapiens LEM domain containing 1 (LEMD1), Homo sapiens similar to GAGE-2 protein (G antigen 2) (LOC645037), Homo sapiens similar to microtubule-associated protein 6 isoform 1 (LOC647315), Homo sapiens matrix metallopeptidase 11 (stromelysin 3) (MMP11), Homo sapiens NK2 transcription factor related, locus 5 (Drosophila) (NKX2-5), Homo sapiens parathyroid hormone-like hormone (PTHLH), Homo sapiens sal-like 4 (Drosophila) (SALL4), Homo sapiens small nucleolar RNA, C/D box 56 (SNORD56), Homo sapiens CSAG family, member 3A (CSAG3A), Homo sapiens family with sequence similarity 83, member A (FAM83A), transcript variant 2, Homo sapiens similar to hCG1812074 (LOC100134331), Homo sapiens hypothetical protein LOC642477, transcript variant 2 (LOC642477), Homo sapiens hypothetical protein LOC645099, transcript variant 1 (LOC645099), Homo sapiens similar to TP53TG3 protein, transcript variant 2 (LOC729264), Homo sapiens protocadherin beta 2 (PCDHB2), Homo sapiens peptidase inhibitor 3, skin-derived (SKALP) (PI3), Homo sapiens TP53 target 3 (TP53TG3), Homo sapiens cathepsin L2 (CTSL2), Homo sapiens gremlin 1, cysteine knot superfamily, homolog (Xenopus laevis) (GREM1), Homo sapiens potassium channel, subfamily K, member 17 (KCNK17), transcript variant 1, Homo sapiens kringle containing transmembrane protein 2 (KREMEN2), transcript variant 2, Homo sapiens hypothetical protein LOC100130082, transcript variant 2 (LOC100130082), Homo sapiens hypothetical LOC645682 (LOC645682), Homo sapiens olfactomedin 4 (OLFM4), Homo sapiens one cut homeobox 2 (ONECUT2), Homo sapiens protein phosphatase, EF-hand calcium binding domain 1 (PPEF1), Homo sapiens reprimo-like (RPRML), Homo sapiens wingless-type MMTV integration site family, member 10A (WNT10A), Homo sapiens annexin A13 (ANXA13), Homo sapiens hypothetical protein FLJ22184 (FLJ22184), Homo sapiens laminin, gamma 2 (LAMC2), Homo sapiens mitogen-activated protein kinase 15 (MAPK15), Homo sapiens nucleoporin 210 kDa (NUP210), Homo sapiens asparagine-linked glycosylation 1-like (ALG1L), Homo sapiens guanine nucleotide binding protein (G protein), gamma 4 (GNG4), Homo sapiens harakiri, BCL2 interacting protein (contains only BH3 domain) (HRK), Homo sapiens nuclear factor (erythroid-derived 2)-like 3 (NFE2L3), Homo sapiens tet oncogene 1 (TET1), Homo sapiens septin 3 (SEPT3), Homo sapiens achaete-scute complex homolog 1 (Drosophila) (ASCL1), Homo sapiens BCL2-interacting killer (apoptosis-inducing) (BIK), Homo sapiens chromosome 21 open reading frame 129 (C21orf129), Homo sapiens calpain 12 (CAPN12), Homo sapiens chromobox homolog 8 (Pc class homolog, Drosophila) (CBX8), Homo sapiens chemokine (C—C motif) ligand 20 (CCL20), Homo sapiens chorionic gonadotropin, beta polypeptide 5 (CGB5), Homo sapiens claudin 9 (CLDN9), Homo sapiens chondrosarcoma associated gene 1 (CSAG1), Homo sapiens CSAG family, member 3B (CSAG3B), Homo sapiens cancer/testis antigen family 45, member A1 (CT45A1), Homo sapiens cancer/testis antigen family 45, member A5 (CT45A5), Homo sapiens cancer/testis antigen 2 (CTAG2), Homo sapiens CCCTC-binding factor (zinc finger protein)-like (CTCFL), Homo sapiens endogenous retroviral sequence K, 6 (ERVK6), Homo sapiens family with sequence similarity 133, member A (FAM133A), PREDICTED: Homo sapiens misc_RNA (FLJ39632), Homo sapiens histone cluster 1, H3h (HIST1H3H), Homo sapiens histone cluster 1, H4h (HIST1H4H), Homo sapiens KIAA1199 (KIAA1199), Homo sapiens LINE-1 type transposase domain containing 1 (L1TD1), Homo sapiens LIM homeobox 2 (LHX2), Homo sapiens hypothetical protein LOC100132564 (LOC100132564), Homo sapiens hypothetical LOC400879, transcript variant 2 (LOC400879), Homo sapiens hypothetical protein LOC643272 (LOC643272), Homo sapiens similar to CSAG family, member 2 (LOC653297), Homo sapiens hypothetical LOC729669 (LOC729669), Homo sapiens mesothelin (MSLN), Homo sapiens NLR family, pyrin domain containing 7 (NLRP7), Homo sapiens one cut homeobox 2 (ONECUT2), Homo sapiens proprotein convertase subtilisin/kexin type 1 (PCSK1), Homo sapiens pancreatic and duodenal homeobox 1 (PDX1), Homo sapiens pregnancy specific beta-1-glycoprotein 1 (PSG1), Homo sapiens serpin peptidase inhibitor, clade A (alpha-1 antiproteinase, antitrypsin), member 1 (SERPINA1), Homo sapiens synaptonemal complex protein 2 (SYCP2), Homo sapiens tudor domain containing 5 (TDRD5), Homo sapiens urotensin 2 domain containing (UTS2D), Homo sapiens WD repeat domain 66 (WDR66), Homo sapiens X antigen family, member 1B (XAGE1B), RC2-CT0321-110100-013-c08 CT0321 Homo sapiens cDNA, Homo sapiens mutS homolog 5 (E. coli) (MSH5), Homo sapiens Mdm2, transformed 3T3 cell double minute 2, p53 binding protein (mouse) binding protein, 104 kDa (MTBP), Homo sapiens collagen, type XI, alpha 1 (COL11A1), Homo sapiens docking protein 7 (DOK7), Homo sapiens fibroblast growth factor 11 (FGF11), Homo sapiens glutamate decarboxylase 1 (brain, 67 kDa) (GAD1), Homo sapiens NORMA domain containing 1 (HORMAD1), Homo sapiens melanoma antigen family A, 12 (MAGEA12), Homo sapiens matrix metallopeptidase 7 (matrilysin, uterine) (MMP7), Homo sapiens NLR family, pyrin domain containing 7 (NLRP7), Homo sapiens NOL1/NOP2/Sun domain family, member 5 (NSUN5), Homo sapiens T-box 1 (TBX1), Homo sapiens tumor necrosis factor receptor superfamily, member 6b, decoy (TNFRSF6B), Homo sapiens UDP glucuronosyltransferase 1 family, polypeptide A6 (UGT1A6), Homo sapiens zinc finger protein 280A (ZNF280A), Homo sapiens epiphycan (EPYC), Homo sapiens neuromedin U (NMU), Homo sapiens SPRY domain containing 5 (SPRYD5), Homo sapiens variable charge, X-linked 2 (VCX2), 17000532640995 GRN_ES Homo sapiens cDNA 5, Homo sapiens hypothetical protein LOC651957 (LOC651957), Homo sapiens variable charge, X-linked 3A (VCX3A), Homo sapiens chemokine (C—X—C motif) receptor 3 (CXCR3), Homo sapiens histone cluster 1, H2am (HIST1H2AM), Homo sapiens kinesin family member 24 (KIF24), Homo sapiens chromosome 3 open reading frame 32 (C3orf32), Homo sapiens interleukin 8 (IL8), Homo sapiens small nucleolar RNA, H/ACA box 72 (SNORA72), Homo sapiens neurotensin (NTS), Homo sapiens protein phosphatase 1E (PP2C domain containing) (PPM1E), Homo sapiens transmembrane 4 L six family member 19, transcript variant 2 (TM4SF19), Homo sapiens baculoviral IAP repeat-containing 7 (BIRC7), Homo sapiens neurexophilin 4 (NXPH4), Homo sapiens annexin A13 (ANXA13), Homo sapiens apolipoprotein B mRNA editing enzyme, catalytic polypeptide 1 (APOBEC1), Homo sapiens chromosome 1 open reading frame 110 (C1 orf110), Homo sapiens C1q and tumor necrosis factor related protein 3 (C1QTNF3), Homo sapiens CD70 molecule (CD70), Homo sapiens cytochrome c oxidase subunit VIIb2 (COX7B2), Homo sapiens G antigen 12B (GAGE12B), Homo sapiens G antigen 12G (GAGE12G), Homo sapiens glyceraldehyde-3-phosphate dehydrogenase, spermatogenic (GAPDHS), Homo sapiens gametocyte specific factor 1 (GTSF1), Homo sapiens histone cluster 1, H2bj (H1ST1H2BJ), Homo sapiens histone cluster 2, H4a (HIST2H4A), Homo sapiens internexin neuronal intermediate filament protein, alpha (INA), Homo sapiens potassium voltage-gated channel, subfamily H (eag-related), member 6 (KCNH6), Homo sapiens potassium large conductance calcium-activated channel, subfamily M, beta member 2 (KCNMB2), Homo sapiens KIAA1688 protein (KIAA1688), Homo sapiens LIM homeobox 8 (LHX8), Homo sapiens misc_RNA (LOC100131707), Homo sapiens misc_RNA (LOC100133312), Homo sapiens hypothetical protein LOC100133542 (LOC100133542), Homo sapiens similar to keratin 8 (LOC100134794), Homo sapiens misc_RNA (LOC651397), Homo sapiens misc_RNA (LOC728178), Homo sapiens melanoma antigen family A, 1 (directs expression of antigen MZ2-E) (MAGEA1), Homo sapiens melanoma antigen family A, 4 (MAGEA4), Homo sapiens melanoma antigen family A, 6 (MAGEA6), Homo sapiens melanoma antigen family B, 2 (MAGEB2), Homo sapiens melanoma antigen family C, 1 (MAGEC1), Homo sapiens melanoma antigen family C, 2 (MAGEC2), Homo sapiens microtubule-associated protein 1 light chain 3 alpha (MAP1LC3A), transcript variant 2, Homo sapiens mitogen-activated protein kinase kinase kinase kinase 1 (MAP4K1), transcript variant 1, Homo sapiens microRNA 25 (MIR25), Homo sapiens metallothionein-like 5, testis-specific (tesmin) (MTL5), Homo sapiens NADH dehydrogenase (ubiquinone) 1 alpha subcomplex, 4-like 2 (NDUFA4L2), Homo sapiens NLR family, pyrin domain containing 7 (NLRP7), Homo sapiens NOP2/Sun domain family, member 5C (NSUN5C), Homo sapiens odorant binding protein 2B (OBP2B), Homo sapiens P antigen family, member 2 (prostate associated) (PAGE2), Homo sapiens P antigen family, member 5 (prostate associated) (PAGE5), Homo sapiens piccolo (presynaptic cytomatrix protein) (PCLO), Homo sapiens piwi-like 1 (Drosophila) (PIWIL1), Homo sapiens podocalyxin-like 2 (PODXL2), Homo sapiens prion protein 2 (dublet) (PRND), Homo sapiens solute carrier family 45, member 2 (SLC45A2), transcript variant 1, Homo sapiens small nucleolar RNA, C/D box 3A (SNORD3A), Homo sapiens small nucleolar RNA, C/D box 3C (SNORD3C), Homo sapiens small nucleolar RNA, C/D box 3D (SNORD3D), Homo sapiens Sad1 and UNC84 domain containing 1 (SUNC1), Homo sapiens synaptotagmin XIII (SYT13), Homo sapiens tripartite motif family-like 2 (TRIML2), Homo sapiens transient receptor potential cation channel, subfamily M, member 2 (TRPM2), Homo sapiens tubulin, beta 3 (TUBB3), Homo sapiens urothelial cancer associated 1 (non-protein coding) (UCA1), Homo sapiens variable charge, X-linked (VCX), Homo sapiens variably charged X—C (VCX—C), Homo sapiens variable charge, X-linked 2 (VCX2), Homo sapiens variable charge, Y-linked (VCY), Homo sapiens VGF nerve growth factor inducible (VGF), Homo sapiens X antigen family, member 1 (XAGE1), HESC3_{—}16_C05.g1_A036 Human embryonic stem cells Homo sapiens cDNA clone IMAGE:7476876 5 or a complement thereof in the sample obtained from the subject compared to the non-cancerous cell indicates that the subject has cancer.
